(19) United States
(12) Patent Application Publication
(10) Pub. No.: US 2014/0088177 A1
(43) Pub. Date: Mar. 27, 2014

(54) RECOMBINANT VSV FOR THE TREATMENT OF TUMOR CELLS

(71) Applicant: University of Miami, Miami, FL (US)

(72) Inventor: Glen N. Barber, Miami, FL (US)

(73) Assignee: University of Miami, Miami, FL (US)

(21) Appl. No.: 13/963,798

(22) Filed: Aug. 9, 2013

Related U.S. Application Data
(63) Continuation-in-part of application No. 13/724,393, filed on Dec. 21, 2012.
Application No. 13/724,393 is a continuation of application No. 10/194,594, filed on Jul. 11, 2002, now abandoned.
(60) Provisional application No. 60/304,125, filed on Jul. 11, 2001.

(51) Int. Cl. C12N 15/86

(52) U.S. Cl. CPC C12N 15/86
USPC 514/44 R; 435/320.1

(57) ABSTRACT

Vesicular Stomatitis Viruses expressing foreign nucleic acid sequences were highly attenuated but were lytic for abnormal cells, such as cancers, infected cells. Methods of treatment include the administration of these viruses.

### CROSS-REFERENCE TO RELATED APPLICATIONS

This application is a Continuation-in-Part of U.S. patent application Ser. No. 13/724,393, filed Dec. 12, 2012, which claims the benefit of priority to U.S. Provisional Patent Application No. 60/681,903 filed Aug. 10, 2012, and is a Continuation of U.S. patent application Ser. No. 10/194,594, filed Jul. 11, 2002, which claims the benefit of U.S. Provisional Patent Application No. 60/304,125, filed Jul. 11, 2001, the entire contents of which are incorporated herein by reference.

### STATEMENT REGARDING FEDERALLY SPONSORED RESEARCH OR DEVELOPMENT

This invention was made with U.S. government support under Grant Nos. P01CA128115A and CA095924-06 awarded by the National Institutes of Health. The Government has certain rights in the invention.

### FIELD OF THE INVENTION

The present invention generally relates to vesicular stomatitis virus (VSV), methods of producing heterologous proteins in recombinant VSV and the use of recombinant VSV comprising cytokines or suicide genes for the treatment of malignant cells.

### BACKGROUND OF THE INVENTION

Vesicular stomatitis virus (VSV), of the genus, Vesiculovirus, is the prototypic member of the family Rhabdoviridae, and is an enveloped virus with a negative stranded RNA genome that causes a self-limiting disease in live-stock and is essentially non-pathogenic in humans. Balachandran and Barber (2000, IUBMB Life 50: 135-8). Rhabdoviruses have single, negative-strand RNA genomes of 11,000 to 12,000 nucleotides (Rose and Schubert, 1987, Rhabdovirus genomes and their products, in The Viruses: The Rhabdoviruses, Plenum Publishing Corp., NY, pp. 129-166). The virus particles contain a helical, nucleocapsid core composed of the genomic RNA and protein. Generally, three proteins, termed N (nucleocapsid, which encases the genome tightly), P (formerly termed NS, originally indicating nonstructural), and L (large) are found to be associated with the nucleocapsid. An additional matrix (M) protein lies within the membrane envelope, perhaps interacting both with the membrane and the nucleocapsid core. A single glycoprotein (G) species spans the membrane and fom1s the spikes on the surface of the virus particle. Glycoprotein G is responsible for binding to cells and membrane fusion. The VSV genome is the negative sense (i.e., complementary to the RNA sequence (positive sense) that functions as mRNA to directly produce encoded protein), and rhabdoviruses must encode and package an RNA-dependent RNA polymerase in the virion (Baltimore et al., 1970, Proc. Natl. A cad. Sci. USA 66: 572-576), composed of the P and L proteins. This enzyme transcribes genomic RNA to make subgenomic mRNAs encoding the 5-6 viral proteins and also replicates full-length positive and negative sense RNAs. The genes are transcribed sequentially, starting at the 3′ end of the genomes.

The sequences of the VSV mRNAs and genome is described in Gallione et al. 1981, J. Viral. 39:529-535; Rose and Gallione, 1981, J. Virol. 39:519-528; Rose and Schubert, 1987, Rhabdovirus genomes and their products, p. 129-166, in R. R. Wagner (ed.), The Rhabdoviruses. Plenum Publishing Corp., NY; Schubert 15 et al., 1985, Proc. Natl. Acad. Sci. USA 82:7984-7988. WO 96/34625 published Nov. 7, 1996, disclose methods for the production and recovery of replicable vesiculovirus. U.S. Pat. No. 6,168,943, issued Jan. 2, 2001, describes methods for making recombinant vesicul oviruses.

Although most immortalized tissue culture cell lines are permissive to VSV, the virus is sensitive to the antiviral actions of the interferons (IFN). Balachandran and Barber, supra. Primary cells containing PKR and a functional INF system are not strongly permissive to VSV replication. Balachandran et al. (2000, Immunity 13, 129-141) disclose that mice lacking the IFN-inducible double stranded RNA-dependent protein kinase (PKR), are susceptible to VSV infection. That VSV is capable of replicating in a majority of mammalian cell lines, but not well in primary cells unless PKR function or INF signaling is defective, implies that host defense mechanisms required to prevent VSV replication are impaired in cells permissive to the virus, including immortalized and malignant cells. Balachandran and Barber, supra.

VSV oncolytic activity is disclosed in WO 00/62735, published Oct. 26, 2000; Balachandran and Barber, supra; Stojdl et al. (2000, Nature Medicine, vol 6, pages 821-825); Balachandran et al. (2001, J. of Virol. p. 34 73-34 79); WO 01119380, published Mar. 22, 2001; WO 99118799 published Apr. 22, 1999; and WO 00/62735 published Oct. 26, 2000.

There remains a need for the development of compositions and methods for the treatment of tumor cells.

All references and patent publications are hereby incorporated herein by reference in their entirety.

### BRIEF SUMMARY OF THE INVENTION

Embodiments of the invention are directed to attenuated strains of vesicular stomatitis virus engineered to also express one or more foreign nucleic acid sequences. Methods for use in treating cancer and viral infections are provided.

The present invention relates to recombinant vesicular stomatitis virus (VSV) expression constructs (vectors) based on VSV which confer infectivity, replication, transcription, or any combination thereof when the construct is introduced into cells either in vitro or in vivo, with or without a viral particle. The VSV construct is engineered to express one or more heterologous nucleotide sequence(s), especially genes encoding a cytokine, such as for example, interferon or interleukin, or other biologically active molecules, such as for example, heat shock protein gp96, or suicide cassette such as thymidine kinase (TK) or cytosine deaminase, or a tumor suppressor molecule, wherein the tumor suppressor molecule is p53, mutants, variants or complementary DNA (cDNA) sequences thereof. In addition, the VSV can encode a viral gene, for example, human immunodeficiency virus (HIV) gp160 nucleic acid sequence, mutants, variants or complementary DNA (cDNA) sequences thereof. The VSV vector may comprise nucleic acid encoding two or more biologically active proteins, for example, two cytokines, such as an interferon and an interleukin, or two interferons or two interleukins. The two or more cytokines maybe identical or different. The recombinant VSV can be replication-competent or replication-defective. The present invention also relates to methods for producing oncolytic activity in tumor and/or malignant cells comprising administering recombinant VSV vectors comprising nucleic acid encoding a cytokine, including for example, interferon, such as, interferon-beta or interferon-gamma, and interleukin, such as, interleukin 4 or interleukin 12 to the tumor and/or malignant cells.

The present invention provides recombinant vesicular stomatitis virus (VSV) vectors comprising nucleic acid encoding a cytokine(s); wherein said recombinant VSV vector exhibits greater oncolytic activity against a tumor cell than a wild-type VSV vector when contacted with the tumor cell. In some examples, the cytokine is an interferon, such as interferon-beta or interferon-gamma. In other examples, the cytokine is an interleukin such as for example, IL-4 or IL-12. In some examples, the VSV vector comprises nucleic acid encoding two or more cytokines, such as two interferons or two interleukins or an interferon, such as interferon-beta and an interleukin, such as interleukin-12. The two or more cytokines maybe identical or different. In additional examples, the VSV vector is replication-defective. In yet other examples, the VSV vector lacks G-protein function and may also lack M and/or N protein function(s). In other examples, the tumor cell is a melanoma tumor cell mammary tumor cell, prostate tumor cell, cervical tumor cell, hematological-associated tumor cell or a cell harboring a defect in a tumor suppressor pathway. The present invention also provides a replication-defective VSV vector comprising nucleic acid encoding interferon, wherein said recombinant VSV vector exhibits greater oncolytic activity against a tumor cell than a wild-type VSV vector when contacted with the tumor cell. In some examples, the interferon is interferon-beta or interferon-gamma. In other examples, the VSV vector lacks G-protein function. In further examples, the tumor cell includes a melanoma tumor cell, mammary tumor cell, prostate tumor cell, cervical tumor cell, hematological-associated tumor cell or a cell harboring a defect in a tumor suppressor pathway. In further examples, an animal comprises the tumor cell and in other examples, the animal is a mammal, such as a human. The present invention also provides viral particles comprising a VSV vector of the present invention, such as a VSV vector comprising nucleic acid encoding a cytokine or suicide gene.

The present invention also comprises isolated nucleic acid encoding a recombinant VSV vector of the present invention as well as host cells comprising a recombinant VSV vector of the present invention. The present invention also provides methods for making a recombinant VSV vector of the present invention comprising growing a cell comprising said VSV vector under conditions whereby VSV is produced; and optionally isolating said VSV. In some examples, the VSV vector is replication defective and the host cells comprise the VSV protein function essential for VSV replication such that said VSV vector is capable of replication in said host cell. In some examples, the VSV vector comprises nucleic acid encoding a cytokine, such as an interferon or interleukin; a suicide gene, such as thymidine kinase or cytosine deaminase or other biological protein, such as a heat shock protein, such as for example, gp96.

The present invention also provides compositions comprising a VSV vector or viral particle of the present invention. In some examples, the VSV vector is present in the composition in an amount effective to produce oncolytic activity in a tumor cell when said composition is contacted with the tumor cell. In other examples the composition comprises a pharmaceutically acceptable excipient.

The present invention also provides methods for producing oncolytic activity in a tumor cell, comprising the step of contacting the cell with a recombinant VSV vector comprising nucleic acid encoding a cytokine, wherein said VSV vector exhibits greater oncolytic activity against the tumor cell than a wild-type VSV vector. In some examples of the methods, the VSV vector is replication-defective. In other examples, the VSV vector lacks G-protein function. In yet further examples, the cytokine is an interferon, such as for example, interferon-beta or interferon-gamma; or a cytokine, such as for example, an interleukin, such as interleukin-4 or interleukin-12. In additional examples, the tumor cell includes a melanoma tumor cell, mammary tumor cell, prostate tumor cell, cervical tumor cell, hematological-associated tumor cell or cell harboring defects in a tumor suppressor pathway. In yet further examples, said contacting is by intravenous injection to an individual comprising said tumor cell or by intra-tumoral injection to an individual comprising said tumor cell.

The present invention also provides methods for producing oncolytic activity in a tumor cell, comprising the step of contacting the tumor cell with a recombinant VSV vector comprising nucleic acid encoding a suicide gene wherein said VSV vector exhibits greater oncolytic activity against the tumor cell when administered along with a prodrug than a wild-type VSV vector. In some examples of the methods, the suicide gene encodes thymidine kinase (TK) and the prodrug is ganciclovir or acyclovir. In other examples, the suicide gene encodes a cytosine deaminase and the prodrug is 5-fluorocytosine. In some examples of the methods, the VSV vector is replication-defective. In other examples, the VSV vector lacks G-protein function. In yet other examples of the methods, the tumor cell includes melanoma tumor cell, mammary tumor cell, prostate tumor cell, cervical tumor cell, hematological-associated tumor cell or cell harboring a defect in a tumor suppressor pathway. In other examples, the contacting is by intravenous injection to an individual comprising said tumor cell or by intratumoral injection to an individual comprising said tumor cell.

The present invention also provide methods for suppressing tumor growth, comprising the step of contacting the tumor with a recombinant VSV vector comprising nucleic acid encoding a cytokine, wherein said VSV vector exhibits greater tumor suppression than a wild-type VSV vector. In some examples of the methods, the VSV vector is replication-defective. In other examples, the VSV vector lacks G-protein function. In yet further examples, the cytokine is an interferon, such as for example, interferon-beta or interferon-gamma; or a cytokine, such as for example, an interleukin, such as interleukin-4 or interleukin-12. The present invention also provides methods for suppressing tumor growth, comprising the step of contacting the tumor with a recombinant VSV vector comprising nucleic acid encoding a suicide gene wherein said VSV vector exhibits greater tumor suppression when administered along with a prodrug than a wild-type VSV vector. In some examples of the methods, the VSV vector is replication-defective. In other examples, the VSV vector lacks G-protein function. In yet further examples, the suicide gene encodes thymidine kinase and the prodrug is ganciclovir or acyclovir. In other examples, the suicide gene encodes a cytosine deaminase and the prodrug is 5-fluorocytosine. In yet other examples of the methods, the tumor cell includes melanoma tumor cell, mammary tumor cell, prostate tumor cell, cervical tumor cell, hematological-associated tumor cell or cell harboring a defect in a tumor suppressor pathway.

The present invention also provides methods for eliciting an immune response to a tumor cell in an individual comprising, administering a composition comprising tumor cells infected with or lysed by a VSV vector to said individual. In some examples, the VSV vector comprises nucleic acid encoding a cytokine, a heat shock protein or an immunomodulatory protein. In some examples, the cytokine is an interferon, such as interferon-beta or interferon-gamma, or interleukin, such as interleukin-4 or interleukin-12. The present invention also provides a composition capable of inducing an immune response in an individual comprising, tumor cells infected with or lysed by a VSV vector. In some examples, the VSV vector comprises nucleic acid encoding a cytokine, a heat shock protein or an immunomodulatory protein. The present invention also contacting a tumor cell obtained from an individual with a VSV vector under conditions suitable for lysing said tumor cells; and returning said lysed tumor cells to said individual. In some examples, the VSV vector comprises nucleic acid encoding a cytokine, a heat shock protein or an immunomodulatory protein.

In other embodiments, a composition comprises a recombinant vesicular stomatitis (VSV) vector encoding a human immunodeficiency virus (HIV) gp160 nucleic acid sequence, mutants, variants or complementary DNA (cDNA) sequences thereof. Preferably, the VSV further comprises one or more foreign nucleic acid sequences or complementary DNA (cDNA) sequences thereof, for expressing one or more proteins or peptides. In preferred embodiments, the one or more foreign nucleic acid sequences or complementary DNA (cDNA) sequences thereof, comprise: interferons, ligands, HIV or simian immunodeficiency virus (SIV) molecules, immune related molecules, cytokines, chemokines, cellular products, cell surface expression or secretion products, cell signaling molecules, or combinations thereof. In embodiments, the immune related molecules comprise: T cell receptors or ligands thereof, co-stimulatory molecules or ligands thereof, immunoglobulins, cell surface expression markers or receptors thereof. In other embodiments, the interferons, cytokines, chemokines and cellular products comprise: interferon α-, β- and/or γ-, interleukins, growth factors, tumor necrosis factors, integrins, or combinations thereof.

In yet other embodiments, the VSV vector further comprises one or more deletions or mutation in one or more VSV nucleic acid sequences and the vector is oncolytic.

In other embodiments, a method of preventing or treating human immunodeficiency virus (HIV) comprising: administering to an at risk patient or a patient suffering from HIV and related disorders, a therapeutically effective amount of a composition comprising: a recombinant vesicular stomatitis (VSV) vector encoding a human immunodeficiency virus (HIV) gp160 nucleic acid sequence, mutants, variants or complementary DNA (cDNA) sequences thereof. In some embodiments, the VSV further comprises one or more foreign nucleic acid sequences or complementary DNA (cDNA) sequences thereof, for expressing one or more proteins or peptides, comprising: interferons, ligands, HIV or simian immunodeficiency virus (SIV) molecules, immune related molecules, cytokines, chemokines, cellular products, cell surface expression or secretion products, cell signaling molecules, or combinations thereof.

In other embodiments, a method of treating cancer comprises administering to a patient in need thereof, a therapeutically effective amount of a composition comprising: a recombinant vesicular stomatitis (VSV) vector encoding a human immunodeficiency virus (HIV) gp160 nucleic acid sequence, mutants, variants or complementary DNA (cDNA) sequences thereof. In embodiments, the VSV further comprises one or more foreign nucleic acid sequences or complementary DNA (cDNA) sequences thereof, for expressing one or more proteins or peptides, comprising: interferons, ligands, HIV or simian immunodeficiency virus (SIV) molecules, immune related molecules, cytokines, chemokines, cellular products, cell surface expression or secretion products, cell signaling molecules, or combinations thereof.

In embodiments, a composition comprises a recombinant vesicular stomatitis (VSV) vector expressing a tumor suppressor molecule, wherein the tumor suppressor molecule is p53, mutants, variants or complementary DNA (cDNA) sequences thereof. In preferred embodiments, the VSV vector is oncolytic. In embodiments, the VSV vector is attenuated. In other embodiments, the VSV vector further comprises one or more foreign nucleic acid sequences or complementary DNA (cDNA) sequences thereof, for expressing one or more proteins or peptides. Preferably, the one or more foreign nucleic acid sequences or complementary DNA (cDNA) sequences thereof, comprise: interferons, ligands, immune related molecules, cytokines, chemokines, cellular products, cell surface expression or secretion products, cell signaling molecules, or combinations thereof.

In other embodiments, a method of treating cancer comprises administering to a patient in need thereof, a therapeutically effective amount of a recombinant vesicular stomatitis (VSV) vector expressing a tumor suppressor molecule, wherein the tumor suppressor molecule is p53, mutants, variants or complementary DNA (cDNA) sequences thereof. Preferably the VSV vector is oncolytic. In other embodiments, the VSV vector is attenuated. In embodiments, the VSV vector further comprises one or more foreign nucleic acid sequences or complementary DNA (cDNA) sequences thereof, for expressing one or more proteins or peptides. In preferred embodiments, the one or more foreign nucleic acid sequences or complementary DNA (cDNA) sequences thereof, comprise: interferons, ligands, immune related molecules, cytokines, chemokines, cellular products, cell surface expression or secretion products, cell signaling molecules, or combinations thereof.

In other embodiments, a composition comprises an attenuated vesicular stomatitis (VSV) vector expressing a one or more oligonucleotides which modulate expression or function of target molecules. In embodiments, the oligonucleotides comprises: dsRNA, siRNA, antisense RNA, RNA, enzymatic RNA or microRNA.

Preferably, the VSV vector is oncolytic or kills abnormal cells. In other embodiments, the VSV vector is attenuated. In other embodiments, the VSV vector further comprises one or more foreign nucleic acid sequences or complementary DNA (cDNA) sequences thereof, for expressing one or more proteins or peptides, the one or more foreign nucleic acid sequences comprising: interferons, ligands, immune related molecules, cytokines, chemokines, cellular products, cell surface expression or secretion products, cell signaling molecules, or combinations thereof.

The present invention also provides kits comprising a VSV vector comprising nucleic acid encoding a cytokine or a suicide gene and instructions for use of the VSV vector.

Also disclosed are methods of making and using VSV constructs to express a cytokine or other biologically active molecule, such as thymidine kinase or cytosine deaminase, as well as viruses or mammalian cells comprising the VSV expression construct. The present invention also provides methods of producing difficult to make, toxic or rare proteins. The present invention also provides methods for producing eukaryotic proteins using VSV expression systems which provide authentic (i.e., eukaryotic) processing.

### BRIEF DESCRIPTION OF THE DRAWING(S)

FIGS. 1A-1B illustrate the generation of recombinant VSV expressing TK, IL-4, IFN, or green fluorescent protein (GFP).

FIG. 1A. cDNA representing the VSV genome (pVSV-XN2), flanked by the T7 RNA polymerase leader and T7 terminator as well as hepatitis virus delta ribozyme (RBZ) was used to create recombinant viruses. IL-4, TK or GFP were inserted between the G and L genes of VSV. FIG. 1B. Growth curves of recombinant viruses. BHK cells were infected with wild type (WT) VSV, VSV-IL-4 and VSV-TK at an m.o.i. of 10. Supernatants from infected cells were harvested at the indicated times post-infection and viral titers determined by plaque assay.

FIGS. 2A-2D illustrate expression of IL-4 or TK from rVSV. FIG. 2A. GCV is phosphorylated in cells infected with VSV-TK. BHK cells were mock infected or infected with VSV-TK or WT VSV (m.o.i.=1) for 8 h and cell lysates were assayed for GCV phosphorylation, in vitro. BHK cells transiently transfected with CMV promoter driven HSV-TK [BHK(+)] or empty vector (BHK) were used as controls. FIG. 2B. Expression of HSV-TK in VSV-TK infected cells. BHK cells were mock infected (lane 1) or infected with WT VSV (lane 2) or VSV-TK (lane 3), at an m.o.i. of 1, for 24 h and cell extracts analyzed for TK expression using an anti-TK monoclonal antibody. 293T cells transiently transfected with an empty vector (lane 4) or CMV promoter driven HSV-TK (lane 5) were used as a positive control. FIG. 2C. High level expression of IL-4 in cells infected with VSV-IL-4. Culture medium from BHK cells infected with WT VSV or VSV-IL-4 was measured for functional IL-4 using capture ELISA. As further controls, IL-4 was measured in culture medium from BHK cells transiently transfected with an empty vector or CMV-promoter driven IL-4 cDNA. FIG. 2D. Immunoprecipitation of IL-4 from supernatants of VSV-IL-4 infected cells. Extracts from [³⁵S]methionine-labeled cells mock infected (lane 1) or infected with VSV-IL-4 (lane 2) or WT VSV (lane 3) were immunoprecipitated with an IL-4 antibody.

FIGS. 3A-3F illustrate the in vitro effects of wild type and recombinant VSVs on primary or transformed cells. FIGS. 3A-3C illustrate efficient replication of VSV-GFP in transformed cells HMVEC, B 16(F10) or DA-3 cells were infected with VSV-GFP with or without prior treatment of lFNα (500 u/ml). Top panels show cells under brightfield microscopy (magnification, 20×) and lower panels shows the same field by immunofluorescence. FIGS. 3D-3F illustrate that rVSVs efficiently kill transformed cells. HMVEC, B16(F10) or DA-3 cells were infected with WT VSV, VSV-TK or VSV-IL-4 with (solid columns) or without (clear columns) prior treatment with IFNα. Cell viability was assayed by Trypan Blue exclusion 18 h after infection.

FIG. 4A-4C illustrate that rVSV expressing TK and IL-4 inhibit the growth of syngeneic breast and melanoma tumors in immunocompetent mice. FIG. 4A. C57B1/6 mice were implanted subcutaneously with a 5×10⁵ B 16(F10) melanoma cells. After palpable tumors had formed, animals were treated intratumorally with 2×10⁷ p.f.u. WT VSV, VSV-IL-4 or VSV-TK. Injections of virus were repeated after 3 days. Tumor volumes were calculated and are shown as a mean±S.E.M. (n=5). Two mice that received heat inactivated virus were sacrificed at day 4 due to the large size of tumors. FIG. 4B. BALB/c mice were implanted subcutaneously with 1.5×10⁶ D1 DMBA3 tumor cells. After palpable tumors had formed animals were intratumorally injected with 2×10⁷ p.f.u. heat inactivated virus, WT VSV, VSV IL-4 or VSV-TK. Virus treatment was repeated after 3 days. Tumor volumes at day 21 post-implantation (7 days after the last virus treatment) are shown. Results 20 are presented as a mean±S.E.M. (n=5). Comparable results were obtained in three independent sets of experiments. FIG. 4C. Induction of CTL response against B16(F10) tumor in animals receiving VSV-TK/GCV treatment. C57B1/6 tumor bearing mice were injected intratumorally with wild type or recombinant VSVs. A second injection was administered 3 days later. Ten days after the first virus injection, spleen cells were isolated and cocultured with B16(F10) cells. Spleen cells were incubated at the indicated effector to target ratios with ⁵¹Cr labeled B16(F10) target cells. CTL activity was detemlined by ⁵¹Cr release.

FIGS. 5A-5D illustrate the histopathological analysis of tumors. Tumors from C57B1/6 and Balb/c mice were removed 7 days after receiving intratumoral injections of either FIG. 5A heat inactivated (HI)-VSV, FIG. 5B WT VSV, FIG. 5C VSV-IL-4, or FIG. 5D VSV-TK. The left panel indicates large areas of cell death in B16(F10) tumors from WT VSV treated tumors, which are more pronounced in tumors treated with VSV-TK and VSV-IL-4. The right panel emphasizes increased infiltration of eosinophils in D1 DMBA3 tumors injected with VSV-IL-4.

FIG. 6 illustrates the genomic organization of the VSV genome.

FIGS. 7A-7B demonstrate that several human cancer cell lines are permissive to VSV replication and lysis. FIG. 7A demonstrates that MCF-7, BC-1, Jurkat, HL60, K562, PC-3 and HeLa cells were treated with or without 1000 U/ml hIFN-β for 18 hours and subsequently infected with VSV. 48 hours post infection, viability was assessed by Trypan blue exclusion analysis. FIG. 7B. Supernatants from cells treated as in FIG. 7 A were analyzed for viral yield by standard plaque assay.

FIG. 8 shows the growth curve of recombinant viruses expressing interferon in BHK-21 cells.

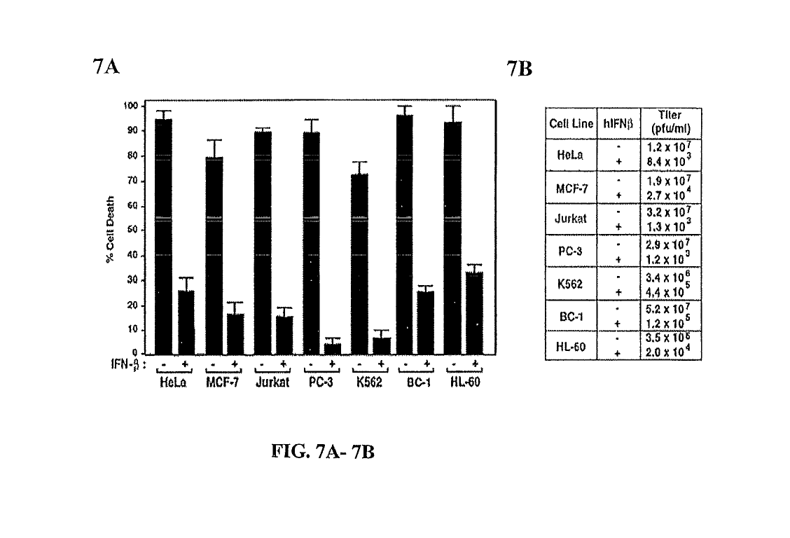

FIG. 9 shows the in vitro effects of VSV-IFN on DA-3 cells at 24 hours after infection.

FIG. 10 shows the production of IFN-beta in recombinant virus-infected BHK-21 cells.

FIGS. 11A-11B show the effects of viral inoculation on weights and survivals.

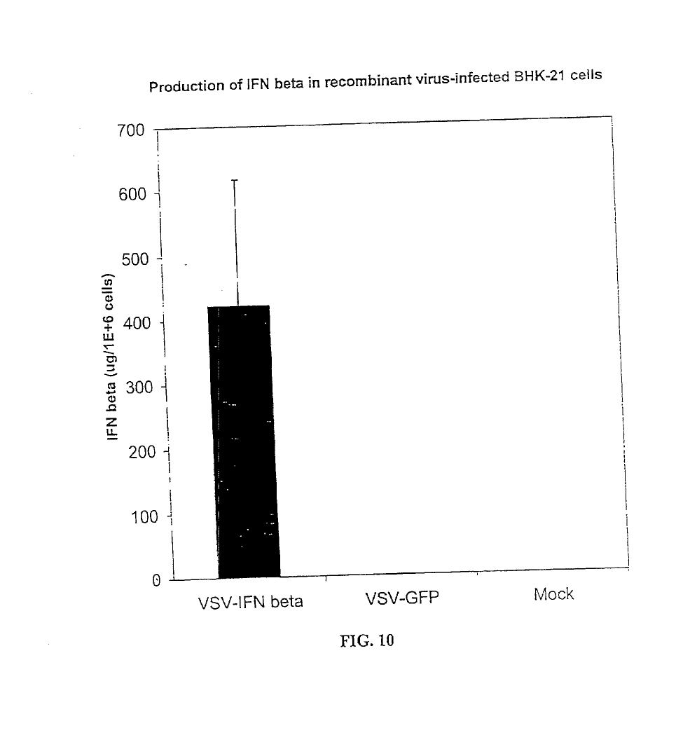

FIG. 11A shows the average weight of mice following virus inoculation. BALB/c mice (n=5 per group) were inoculated intravenously with VSV-IFNβ, VSV-GFP, or rVSV at 5×10⁶ or 2×10⁷ p.f.u. per mouse, and weights of mice were measured every week. Error bars show 0.5× standard deviation. FIG. 11B shows the survival rate of mice following virus inoculation. BALB/c mice (n=5 per group) were inoculated intravenously with 1×10⁸ p.f.u. of VSV-IFNβ, VSV-GFP, or wild-type VSV, and the mortality of mice was monitored daily.

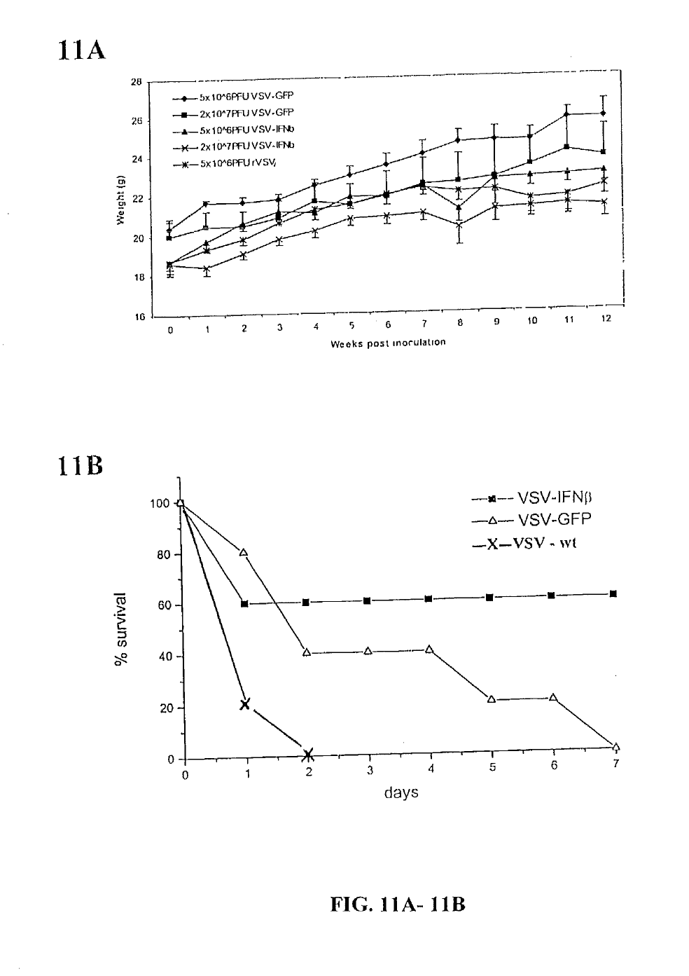

FIG. 12 shows IL-12 expression by BHK cells infected VSV-IL-12.

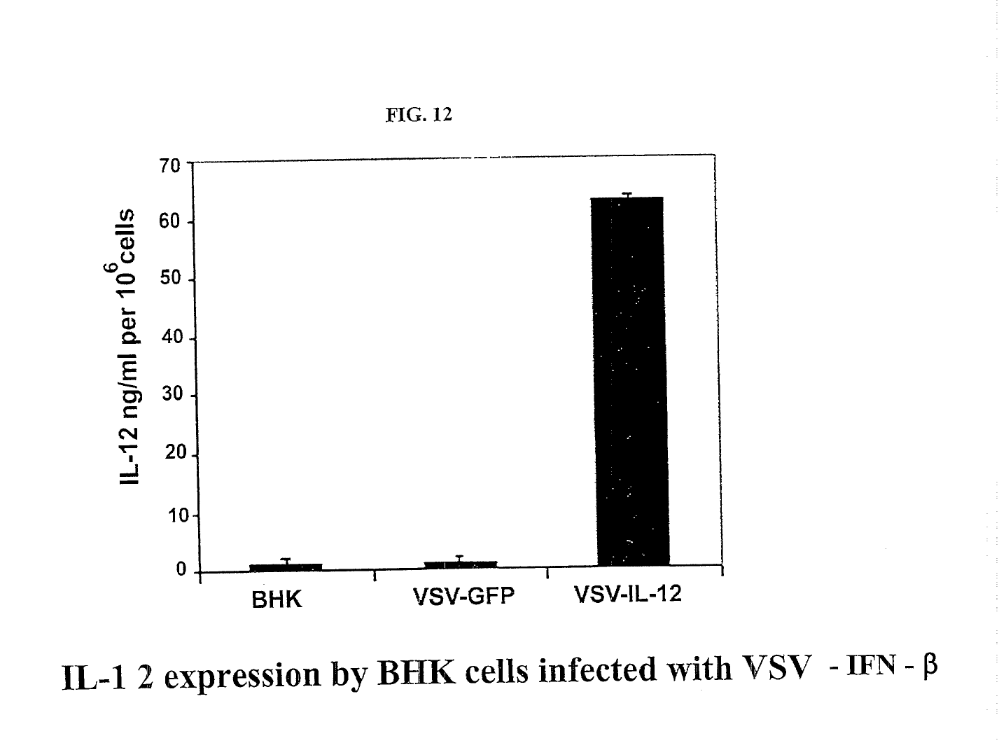

FIG. 13 shows expression of gp96 in BHK cells that are not infected or infected with VSV-gp96 or VSV GFP (m.o.i. 10).

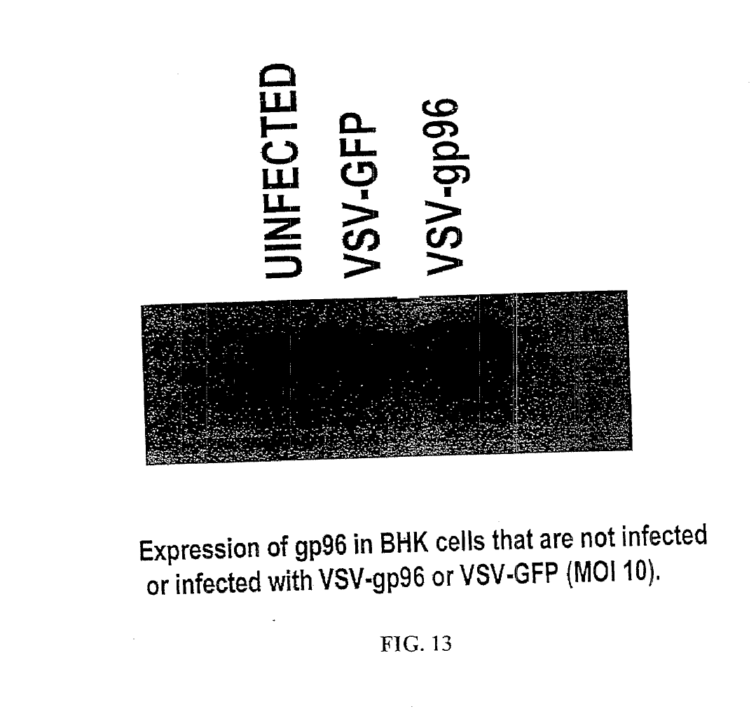

FIG. 14 shows expression of endostatin in cell lysates and supernatants of BHK cells infected with VSV-endostatin:angiostatin or VSV-GFP at an m.o.i. of 10. As a positive control, cells were transfected with a plasmid expressing endostatin:angiostatin (pBlast:mEndo:Angio).

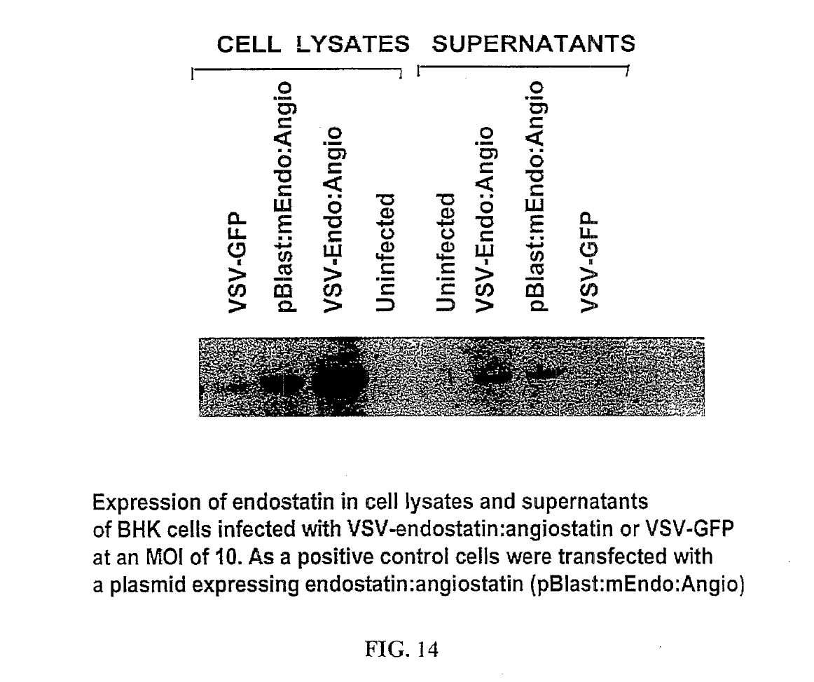

FIGS. 15A-15C show that VSV-M(mut)-mp53 and VSV-mp53 retain oncolytic ability and express mp53. FIG. 15A: Schematic representation of VSV p53 constructs. FIG. 15B: Bright-field microscopy of VSV-M(mut)-mp53- and VSV-mp53-infected cells at MOIs of 0.01 and 5 24 h postinfection. Mock, mock infection. FIG. 15C: Immunoblot analysis for VSV and mp53 protein expression in infected C57BL/6 MEFs and TS/A or B16(F10) cells infected at MOIs of 0.01, 0.1, 1, and 5 24 h postinfection.

FIGS. 16A-16F show VSV-M(mut)-mp53 and VSV-mp53 replication in vitro. C57BL/6 MEFs and TS/A or B16(F10) cells were infected with rVSVs at an MOI of 0.1, 1, or 5. Cells and supernatants were collected 6, 12, 18, and 24 h postinfection. (FIGS. 16A, 16B, and 16C) Cell death at each time point as determined by annexin V-propidium iodide staining. (FIGS. 16D, 16E, and 16F) Supernatants were analyzed by the standard plaque assay to determine the replication of rVSVs. (Two-way analysis of variance [ANOVA]/Bonferroni posttest: *, P<0.001; **, P<0.0001; blue asterisks, VSV-M(mut)-mp53 versus VSV-mp53; orange asterisks, VSV-M(mut)-mp53 versus VSV-ΔM; green asterisks, VSV-M(mut)-mp53 versus VSV-M(mut)-GFP; black asterisks, VSV-M(mut)-mp53 versus VSV-GFP). The error bars indicate standard deviations.

FIGS. 17A-17C show that expressed mp53 is functional. FIG. 17A: C57BL/6 MEFs and TS/A or B16(F10) cells were infected with rVSV at MOIs of 0.1 and 5. The cells were lysed 24 h postinfection (pi) and used for immunoblot analysis. FIG. 17B: C57BL/6 MEFs or TS/A cells were transfected with a p53 luciferase reporter and then infected at an MOI of 0.1 or 5. Luciferase levels were assessed 24 h postinfection. (*, P=0.0366; one-way ANOVA.) The error bars indicate standard deviations. FIG. 17C: C57BL/6 MEFs or TS/A cells were infected at an MOI of 10 or 1, respectively, and mp53 target gene mRNA expression was determined.

FIGS. 18A-18C VSV-M(mut)-mp53 protects immunocompetent mice from TS/A-luc formation. FIG. 18A: Mock- or rVSV-treated mice were anesthetized and injected intraperitoneally (i.p.) with a luciferin substrate, and luciferase activity was detected using the IVIS. Representative images from mock-, VSV-M(mut)-mp53-, and VSV-mp53-treated mice are shown. Luciferase radiance is an indicator of tumor burden and is quantified using Living Image software (right). FIG. 18B: BALB/c mice (n=7) were initially given 1×10⁵ TS/A-luc cells i.v. on day 0 and were then either mock or rVSV treated [5×10⁷ PFU or 5×10⁸ PFU VSV-M(mut)-mp53 only] on day 3. The mice were then monitored for survival (*, P=0.0045; log rank test). FIG. 18C: Previously treated or naïve mice (n=4) were challenged with 2×10⁵ TS/A-luc cells on day 120 (day 0 for rechallenge), and survival was monitored (*, P=0.0091; log rank test).

FIGS. 19A-19C show that VSV-M(mut)-mp53 modulates T cells and is attenuated in athymic BALB/c nude mice. FIG. 19A: BALB/c mice (n=3) were infected with 5×10⁷ PFU rVSV; 96 h postinfection, the spleens were removed and total splenocytes were assessed for the percentage of CD8⁺ cells by flow cytometry (*, P=0.0069; one-way ANOVA). FIG. 19B: BALB/c mice (n=6) were initially given 1×10⁵ TS/A-luc cells i.v. on day 0 and were then either mock treated or treated with 5×10⁷ PFU rVSV on day 3. On day 13, the mice were sacrificed, and total splenocytes were used for an IFN-γ ELISPOT with mitomycin C-treated TS/A-luc cells as the target (*, P<0.0001; one-way ANOVA). Horizontal lines indicate means of results. FIG. 19C: Athymic BALB/c nude mice (n=5) were injected with 1×10⁵ TS/A-luc cells on day 0, mock treated or treated with 5×10⁷ PFU rVSV on day 3, and then observed for survival (P=0.2077; log rank test).

### DETAILED DESCRIPTION OF THE INVENTION

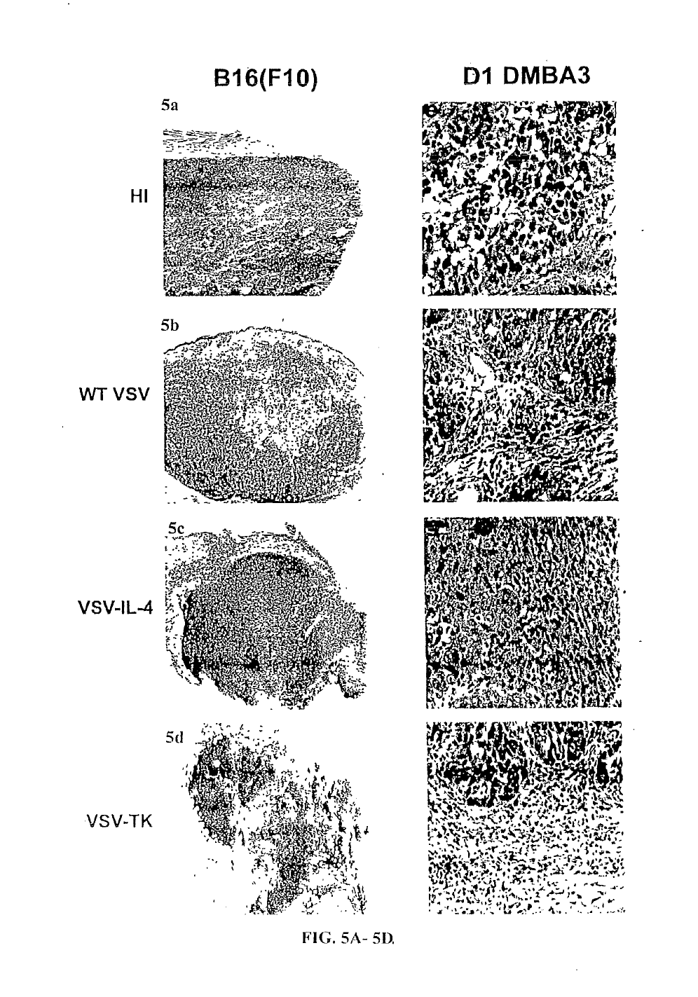

Vesicular stomatitis virus (VSV) is a negative-stranded virus, comprising only 5 genes, that preferentially replicates in immortalized and malignant cells, eventually inducing apoptosis. A schematic illustration of the VSV viral genome is shown in FIG. 6. The ability of VSV to reproduce in tumor or malignant cells has been reported to occur, in part, to a defective interferon (IFN) system. Since the IFN system is functional in normal cells, efficient replication of VSV, which is an IFN-sensitive virus, is prevented. Based on in vitro and in vivo observations, it has been demonstrated that VSV effectively replicates in and lyses infected cancer cells, while leaving normal cells relatively unaffected. Stodj et al., supra; Fernandez et al. (2002, J. Virol. 76:895-904); Balachandran et al. (2001, J. Virol, 75:3474-9; Balachandran and Barber supra.

The use of VSV as an oncolytic agent has several advantages over other virus delivery systems presently used in tumor therapy such as adenoviruses and retroviruses. Foremost, VSV has no known transforming abilities. VSV is not gene-attenuated, which affects replication and therefore oncolytic anti-tumor activity. The envelope glycoprotein (G) of VSV is highly tropic for a number of cell-types and should be effective at targeting a variety of tissues in vivo. VSV appears to be able to replicate in a wide variety of tumorigenic cells and not, for example, only in cells defective in selective tumor suppressor genes such as p53. VSV is able to potently exert its oncolytic activity in tumors harboring defects in the Ras, Myc and p53 pathways, cellular aberrations that occur in over 90% of all tumors. VSV can be modified through genetic engineering to comprise immunomodulatory and/or suicide cassettes designed to increase the anti-tumor activity of the VSV.

Results of experiments disclosed herein demonstrate that a VSV vector comprising nucleic acid encoding a cytokine or suicide cassette exhibits greater oncolytic activity against tumor cells than a wild-type VSV vector alone. Results from experiments disclosed herein demonstrate that a VSV vector comprising 5 nucleic acid encoding TK exhibits oncolytic activity against systemic and subcutaneous tumors and stimulates anti-tumor T-cell response. Data also demonstrate that VSV-IL4 or VSV-TK induce apoptosis, in vivo, of highly aggressive melanoma cells when an animal is infected at an m.o.i. of 1 or less. The data also demonstrate that VSV-TK and VSV-IL-4 exhibit oncolytic activity superior to VSV alone in examples disclosed herein.

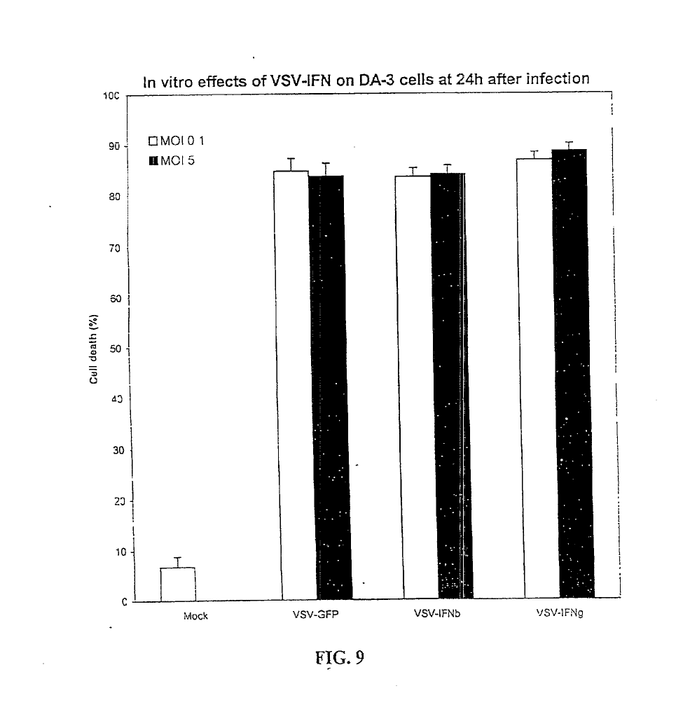

VSV has also been successfully used to express IFN-beta (FIG. 10) or IFN-gamma VSV-IFN can be grown in tumor cells since the IFN response is defective in these cells. Therefore, VSV-IFN still replicates in tumor cells to destroy them. During replication in the tumor cells, VSV makes high levels of IFN, which is secreted to surrounding cells. IFN is a powerful immunostimulatory molecule (these cytokines can activate dendritic cells and NK cells) and they also have tumor suppressive properties. Thus, the synthesis of IFN from VSV-IFN infected cells may induce additional anti-tumor affects and enhance the oncolytic activity of VSV. Normal cells surrounding the tumor should be activated (have their anti-viral state induced) by the VSV-synthesized IFN and become additionally protected against inadvertent VSV infection. In effect VSV-IFN should exert more potent anti-tumor activity than VSV alone and should be safer for normal, that is, non-tumor cells. The data disclosed herein indicate that VSV-IFN kills cancerous cells very efficiently, and normal cells are considerably more protected. Thus, VSV expressing IFN-beta is specific against cancer cells, more attenuated in normal cells, and therefore, safer. Results from experiments described herein demonstrate that a VSV vector comprising nucleic acid encoding IFN-beta or IFN-gamma replicates in cancerous cells and kills them. The data also demonstrate that VSV-IFN-beta and VSV-IFN-gamma exhibit oncolytic activity superior to VSV alone.

Data disclosed herein indicate that VSV vectors can be utilized to express high levels of biologically active recombinant proteins. Essentially, following virus infection, cellular transcription and translation is prevented, and cytoplasmic resources are focused on unbridled expression of the virus genes and any accompanying heterologous nucleic acid, even potentially toxic cellular or viral genes. Data disclosed herein demonstrate that VSV can be modified to deliver genes such as suicide and immunomodulatory cassettes that can greatly increase oncolytic activity, such as for example, killing of tumor cells. In addition, VSV possesses high target specificity and proficient transfection efficacy. Examples include: a recombinant vesicular stomatitis (VSV) vector encoding a human immunodeficiency virus (HIV) gp160 nucleic acid sequence, a tumor suppressor molecule, wherein the tumor suppressor molecule is p53, mutants, variants or complementary DNA (cDNA) sequences thereof.

General Techniques

The practice of the present invention will employ, unless otherwise indicated, conventional techniques of molecular biology (including recombinant techniques), microbiology, cell biology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller 20 & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991).

For general information related to vesicular stomatitis virus, see, “Fundamental Virology”, second edition, 1991, ed. B. N. Fields, Raven Press, New York, pages 489-503; and “Fields Virology”, third edition, 1995, ed. B. N. Fields, vol. 1, pages 1121-1159.

All genes, gene names, and gene products disclosed herein are intended to correspond to homo logs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the molecules disclosed herein, are not limited to a single species but human is preferred, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

“VSV” as used herein refers to any strain of VSV or mutant forms of VSV, such as those described in WO 01/19380. A VSV construct of this invention may be in any of several forms, including, but not limited to, genomic RNA, mRNA, cDNA, part or all of the VSV RNA encapsulated in the nucleocapsid core, VSV complexed with compounds such as PEG and VSV conjugated to a nonviral protein. VSV vectors of the invention encompasses replication-competent and replication-defective VSV vectors, such as, VSV vectors lacking G glycoprotein.

As used herein, the terms “malignant”, “malignant cells”, “tumor”, “tumor cells”, “cancer” and “cancer cells”, (used interchangeably) refer to cells which exhibit relatively autonomous growth, so that they exhibit an aberrant growth phenotype characterized by a significant loss of control of cell proliferation. The term “tumors” includes metastatic as well as non-metastatic tumors.

As used herein “oncolytic activity” refers to inhibition or suppression of tumor and/or malignant and/or cancerous cell growth; regression of tumor and/or malignant and/or cancerous cell growth; cell death of tumor and/or malignant and/or cancerous cells or prevention of the occurrence of additional tumor and/or malignant and/or cancerous cells. As used herein, “inhibiting or suppressing tumor growth” refers to reducing the rate of growth of a tumor, halting tumor growth completely, causing a regression in the size of an existing tumor, eradicating an existing tumor and/or preventing the occurrence of additional tumors upon administration of the VSV comprising compositions, or methods of the present invention. “Suppressing” tumor growth indicates a growth state that is curtailed when compared to growth without contact with a VSV of the present invention. Tumor cell growth can be assessed by any means known in the art, including, but not limited to, measuring tumor size, determining whether tumor cells are proliferating using a ³H-thymidine incorporation assay, or counting tumor cells. “Suppressing” tumor and/or malignant and/or cancerous cell growth means any or all of the following states: slowing, delaying, and stopping tumor growth, as well as tumor shrinkage. “Delaying development” of tumor and/or malignant and/or cancerous cells means to defer, hinder, slow, retard, stabilize, and/or postpone development of the disease. This delay can be of varying lengths of time, depending on the history of the disease and/or individual being treated.

The term “cytokine” as used herein includes any cytokine capable of stimulating an immune response in an individual. Such cytokines include, but are not limited to, interleukins, including but not limited to interleukin-2, interleukin-4, interleukin-6, interleukin-12; interferons, including but not limited to, interferon-alpha, interferon-beta, interferon-gamma, interferon-omega and interferon-epsilon; granulocyte-macrophage colony stimulating factors, and tumor necrosis factor. An “immunomodulatory” protein is one that can stimulate the immune system and includes, but is not limited to cytokines and chemokines.

The term “suicide cassette” or “suicide gene” (interchangeable herein) refer to genes that assist in killing tumor cells and include but are not limited to thymidine: kinase and cytosine deaminase.

As used herein, the term “vector” refers to a polynucleotide construct designed for transduction/transfection of one or more cell types. VSV vectors may be, for example, “cloning vectors” which are designed for isolation, propagation and replication of inserted nucleotides, “expression vectors” which are designed for expression of a nucleotide sequence in a host cell, or a “viral vector” which is designed to result in the production of a recombinant virus or virus-like particle, or “shuttle vectors”, which comprise the attributes of more than one type of vector. The present invention encompasses VSV vectors that comprise nucleic acid encoding cytokines, including but not limited to those cytokines described herein; chemokines, such as for example, Mip; co-stimulatory proteins, such as for example, B7-1 and B7-2; angiostatin; endostatin; heat shock proteins, such as for example gp96, viral proteins e.g. human immunodeficiency virus (HIV) gp160; tumor suppressor molecules, e.g. p53.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, genomic RNA, mRNA, DNA-RNA hybrid, or a polymer comprising purine and pyrimidine bases, or other natural, chemically, biochemically modified, non-natural or derivatized nucleotide bases. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P—NH₂) or a mixed phosphoramidate-phosphodiester oligomer. Peyrottes et al. (1996) Nucleic Acids Res. 24: 1841-8; Chaturvedi et al. (1996) Nucleic Acids Res. 24: 2318-23; Schultz et al. (1996) Nucleic Acids Res. 24: 2966-73. A phosphorothioate linkage can be used in place of a phosphodiester linkage. Braun et al. (1988) J. Immunol. 141:2084-9; Latimer et al. (1995) Molec. Immunol. 32: 1057-1064. In addition, a double-stranded polynucleotide can be obtained from the single stranded polynucleotide product of chemical synthesis either by synthesizing the complementary strand de novo using a DNA polymerase with an appropriate primer. Reference to a polynucleotide sequence (such as referring to a SEQ ID NO) also includes the complement sequence.

The following are non-limiting examples of polynucleotides: a gene or gene fragment, exons, introns, genomic RNA, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

“Under transcriptional control” is a term well understood in the art and indicates that transcription of a polynucleotide sequence depends on its being operably (operatively) linked to an element which contributes to the initiation of or promotes, transcription. “Operably linked” refers to a juxtaposition wherein the elements are in an arrangement allowing them to function. In the context of VSV, a “heterologous polynucleotide” or “heterologous gene” or “transgene” is any polynucleotide or gene that is not present in wild-type VSV.

In the context of VSV, a “heterologous” promoter is one which is not associated with or derived from VSV.

“Expression” includes transcription and/or translation. As used herein, the term “comprising” and its cognates are used in their inclusive sense; that is, equivalent to the term “including” and its corresponding cognates.

As used herein, the term “oligonucleotide specific for” refers to an oligonucleotide having a sequence (i) capable of forming a stable complex with a portion of the targeted gene, or (ii) capable of forming a stable duplex with a portion of a mRNA transcript of the targeted gene.

As used herein, the terms “oligonucleotide,” “siRNA,” “siRNA oligonucleotide,” and “siRNA's” are used interchangeably throughout the specification and include linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoogsteen or reverse Hoogsteen types of base pairing, or the like.

The oligonucleotide may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide in the case of an oligonucleotide compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. The chimeric oligonucleotides of the present invention can be formed as mixed structures of two or more oligonucleotides, modified oligonucleotides, oligonucleotides and/or oligonucleotide analogs as described above.

The oligonucleotide can be composed of regions that can be linked in “register,” that is, when the monomers are linked consecutively, as in native DNA, or linked via spacers. The spacers are intended to constitute a covalent “bridge” between the regions and have in preferred cases a length not exceeding about 100 carbon atoms. The spacers may carry different functionalities, for example, having positive or negative charge, carry special nucleic acid binding properties (intercalators, groove binders, toxins, fluorophors etc.), being lipophilic, inducing special secondary structures like, for example, alanine containing peptides that induce alpha-helices.

As used herein, the term “monomers” typically indicates monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphornates, phosphoroselenoate, phosphoramidate, and the like, as more fully described below.

In the present context, the terms “nucleobase” covers naturally occurring nucleobases as well as non-naturally occurring nucleobases. It should be clear to the person skilled in the art that various nucleobases which previously have been considered “non-naturally occurring” have subsequently been found in nature. Thus, “nucleobase” includes not only the known purine and pyrimidine heterocycles, but also heterocyclic analogues and tautomers thereof. Illustrative examples of nucleobases are adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C₃-C₆)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleobases described in Benner et al., U.S. Pat. No. 5,432,272. The term “nucleobase” is intended to cover every and all of these examples as well as analogues and tautomers thereof. Especially interesting nucleobases are adenine, guanine, thymine, cytosine, and uracil, which are considered as the naturally occurring nucleobases in relation to therapeutic and diagnostic application in humans.

As used herein, “nucleoside” includes the natural nucleosides, including 2′-deoxy and 2′-hydroxyl forms, e.g., as described in Kornberg and Baker, DNA Replication, 2nd Ed. (Freeman, San Francisco, 1992).

“Analogs” in reference to nucleosides includes synthetic nucleosides having modified base moieties and/or modified sugar moieties, e.g., described generally by Scheit, Nucleotide Analogs, John Wiley, New York, 1980; Freier & Altmann, Nucl. Acid. Res., 1997, 25(22), 4429-4443, Toulmé, J. J., Nature Biotechnology 19:17-18 (2001); Manoharan M., Biochemica et Biophysica Acta 1489:117-139 (1999); Freier S. M., Nucleic Acid Research, 25:4429-4443 (1997), Uhlman, E., Drug Discovery & Development, 3: 203-213 (2000), Herdewin P., Antisense & Nucleic Acid Drug Dev., 10:297-310 (2000),); 2′-0,3′-C-linked [3.2.0]bicycloarabinonucleosides (see e.g. N. K Christiensen., et al, J. Am. Chem. Soc., 120: 5458-5463 (1998). Such analogs include synthetic nucleosides designed to enhance binding properties, e.g., duplex or triplex stability, specificity, or the like.

As used herein, the term “gene” means the gene and all currently known variants thereof and any further variants which may be elucidated.

As used herein, “variant” of polypeptides refers to an amino acid sequence that is altered by one or more amino acid residues. The variant may have “conservative”, changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type target gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

As used herein, the term “oligonucleotide specific for” refers to an oligonucleotide having a sequence (i) capable of forming a stable complex with a portion of the targeted gene, or (ii) capable of forming a stable duplex with a portion of a mRNA transcript of the targeted gene.

As used herein, the term “mRNA” means the presently known mRNA transcript(s) of a targeted gene, and any further transcripts which may be elucidated.

By “desired RNA” molecule is meant any foreign RNA molecule which is useful from a therapeutic, diagnostic, or other viewpoint. Such molecules include antisense RNA molecules, decoy RNA molecules, enzymatic RNA, therapeutic editing RNA and agonist and antagonist RNA.

By “antisense RNA” is meant a non-enzymatic RNA molecule that binds to another RNA (target RNA) by means of RNA-RNA interactions and alters the activity of the target RNA (Eguchi et al., 1991 Annu Rev. Biochem. 60, 631-652).

RNA interference “RNAi” is mediated by double stranded RNA (dsRNA) molecules that have sequence-specific homology to their “target” nucleic acid sequences (Caplen, N. J., et al., Proc. Natl. Acad. Sci. USA 98:9742-9747 (2001)). In certain embodiments of the present invention, the mediators of RNA-dependent gene silencing are 21-25 nucleotide “small interfering” RNA duplexes (siRNAs). The siRNAs are derived from the processing of dsRNA by an RNase enzyme known as Dicer (Bernstein, E., et cd., Nature 409:363-366 (2001)). siRNA duplex products are recruited into a multi-protein siRNA complex termed RISC(RNA Induced Silencing Complex). Without wishing to be bound by any particular theory, a RISC is then believed to be guided to a target nucleic acid (suitably mRNA), where the siRNA duplex interacts in a sequence-specific way to mediate cleavage in a catalytic fashion (Bernstein, E., et al., Nature 409:363-366 (2001); Boutla, A., et al., Curr. Biol. 11:1776-1780 (2001)). Small interfering RNAs that can be used in accordance with the present invention can be synthesized and used according to procedures that are well known in the art and that will be familiar to the ordinarily skilled artisan. Small interfering RNAs for use in the methods of the present invention suitably comprise between about 0 to about 50 nucleotides (nt). In examples of nonlimiting embodiments, siRNAs can comprise about 5 to about 40 nt, about 5 to about 30 nt, about 10 to about 30 nt, about 15 to about 25 nt, or about 20-25 nucleotides.

Selection of appropriate oligonucleotides is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of oligonucleotides that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

By “enzymatic RNA” is meant an RNA molecule with enzymatic activity (Cech, 1988 J. American. Med. Assoc. 260, 3030-3035). Enzymatic nucleic acids (ribozymes) act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through base-pairing, and once bound to the correct site, acts enzymatically to cut the target RNA.

By “decoy RNA” is meant an RNA molecule that mimics the natural binding domain for a ligand. The decoy RNA therefore competes with natural binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is meant to be a specific example. Those in the art will recognize that this is but one example, and other embodiments can be readily generated using techniques generally known in the art.

The term “stability” in reference to duplex or triplex formation generally designates how tightly an antisense oligonucleotide binds to its intended target sequence; more particularly, “stability” designates the free energy of formation of the duplex or triplex under physiological conditions. Melting temperature under a standard set of conditions is a convenient measure of duplex and/or triplex stability. It is understood that a stable duplex or triplex may in some embodiments include mismatches between base pairs and/or among base triplets in the case of triplexes. Preferably, modified oligonucleotides, e.g. comprising LNA units, of the invention form perfectly matched duplexes and/or triplexes with their target nucleic acids.

As used herein, the term “Thermal Melting Point (Tm)” refers to the temperature, under defined ionic strength, pH, and nucleic acid concentration, at which 50% of the oligonucleotides complementary to the target sequence hybridize to the target sequence at equilibrium. As the target sequences are generally present in excess, at Tm, 50% of the oligonucleotides are occupied at equilibrium). Typically, stringent conditions will be those in which the salt concentration is at least about 0.01 to 1.0 M Na ion concentration (or other salts) at pH 7.0 to 8.3 and the temperature is at least about 30° C. for short oligonucleotides (e.g., 10 to 50 nucleotide). Stringent conditions may also be achieved with the addition of destabilizing agents such as formamide.

The term “stringent conditions” refers to conditions under which an oligonucleotide will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH.

The term “target nucleic acid” refers to a nucleic acid (often derived from a biological sample), to which the oligonucleotide is designed to specifically hybridize. It is either the presence or absence of the target nucleic acid that is to be detected, or the amount of the target nucleic acid that is to be quantified. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA) whose expression level it is desired to detect. The difference in usage will be apparent from context.

“Target molecule” includes any macromolecule, including protein, carbohydrate, enzyme, polysaccharide, glycoprotein, receptor, antigen, antibody, growth factor; or it may be any small organic molecule including a hormone, substrate, metabolite, cofactor, inhibitor, drug, dye, nutrient, pesticide, peptide; or it may be an inorganic molecule including a metal, metal ion, metal oxide, and metal complex; it may also be an entire organism including a bacterium, virus, and single-cell eukaryote such as a protozoon.

The ability of an oligonucleotide containing a given target sequence to cause RNAi-mediated degradation of the target mRNA can be evaluated using standard techniques for measuring the levels of RNA or protein in cells. For example, RNA of the invention can be delivered to cultured cells, and the levels of target mRNA can be measured by Northern blot or dot blotting techniques, or by quantitative RT-PCR. RNAi-mediated degradation of target mRNA by an siRNA containing a given target sequence can also be evaluated with animal models, such as mouse models. RNAi-mediated degradation of the target mRNA can be detected by measuring levels of the target mRNA or protein in the cells of a subject, using standard techniques for isolating and quantifying mRNA or protein as described above.

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

A “host cell” includes an individual cell or cell culture which can be or has been a recipient of a VSV vector(s) of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected, transformed or infected in vivo or in vitro with a VSV vector of this invention.

“Replication” and “propagation” are used interchangeably and refer to the ability of an VSV vector of the invention to reproduce or proliferate. These terms are well understood in the art. For purposes of this invention, replication involves production of VSV proteins and is generally directed to reproduction of VSV. Replication can be measured using assays standard in the art. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

An “individual” is a vertebrate, preferably a mammal, more preferably a human. Mammals include, but are not limited to, farm animals, sport animals, rodents, primates, e.g. humans, and pets. The terms “patient”, “subject” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Treating” or “treatment” of a state, disorder or condition includes: (1) Preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) Relieving the disease, i.e., causing regression of the state, disorder or condition or at least one of its clinical or sub-clinical symptoms. The benefit to a subject to be treated is either statistically significant or at least perceptible to the patient or to the physician.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

An “effective amount” is an amount sufficient to effect beneficial or desired results, including clinical results. An effective amount can be administered in one or more administrations. For purposes of this invention, an effective amount of a VSV vector is an amount that is sufficient to palliate, ameliorate, stabilize, reverse, slow or delay the progression of the disease state.

As used herein, a “pharmaceutically acceptable” component/carrier etc. is one that is suitable for use with humans and/or animals without undue adverse side effects (such as toxicity, irritation, and allergic response) commensurate with a reasonable benefit/risk ratio.

Vesicular Stomatitis Virus Compositions

VSV Sequences and Constructs:

VSV, a member of the Rhabdoviridae family, is a negative-stranded virus that replicates in the cytoplasm of infected cells, does not undergo genetic recombination or reassortment, has no known transforming potential and does not integrate any part of it genome into the host. VSV comprises an about 11 kilobase genome that encodes for five proteins referred to as the nucleocapsid (N), polymerase proteins (L) and (P), surface glycoprotein (G) and a peripheral matrix protein (M). The genome is tightly encased in nucleocapsid (N) protein and also comprises the polymerase proteins (L) and (P). Following infection of the cell, the polymerase proteins initiate the transcription of five sub genomic viral mRNAs, from the negative-sense genome, that encode the viral proteins. The polymerase proteins are also responsible for the replication of the full-length viral genomes that are packaged into progeny virions. The matrix (M) protein binds to the RNA genome/nucleocapsid core (RNP) and also to the glycosylated (G) protein, which extends from the outer surface in an array of spike like projections and is responsible for binding to cell surface receptors and initiating the infectious process.

Following attachment of VSV through the (G) protein to receptor(s) on the host surface, the virus penetrates the host and uncoats to release the RNP particles. The polymerase proteins, which are carried in with the virus, bind to the 3′ end of the genome and sequentially synthesize the individual mRNAs encoding N, P, M, G, and L, followed by negative-sense progeny genomes. Newly synthesized N, P and L proteins associate in the cytoplasm and form RNP cores which bind to regions of the plasma membrane rich in both M and G proteins. Viral particles form and budding or release of progeny virus ensues.

A schematic illustration of the VSV genome is shown in FIG. 6. A table of various VSV strains is shown in “Fundamental Virology”, second edition, supra, at page 490. WO 01/19380 and U.S. Pat. No. 6,168,943 disclose that strains of VSV include Indiana, New Jersey, Piry, Colorado, Coccal, Chandipura and San Juan. The complete nucleotide and deduced protein sequence of a VSV genome is known and is available as Genbank VSVCG, accession number J02428; NCBI Seq ID 335873; and is published in Rose and Schubert, 1987, in The Viruses: The Rhabdoviruses, Plenum Press, NY. pp. 129-166. A complete sequence of a VSV strain is shown in U.S. Pat. No. 6,168,943. VSV New Jersey strain is available from the American Type Culture Collection (ATCC) and has ATCC accession number VR-159. VSV Indiana strain is available from the ATCC and has ATCC accession number VR-1421.

The present invention encompasses the use of any strain of VSV, including mutants of VSV disclosed in WO 01/19380. The present invention encompasses any form of VSV, including, but not limited to genomic RNA, mRNA, cDNA, and part or all of VSV RNA encapsulated in the nucleocapsid core. The present invention encompasses VSV in the form of a VSV vector construct as well as VSV in the form of viral particles. The present invention also encompasses nucleic acid encoding specific VSV vectors disclosed herein. As discussed herein, VSV vectors of the present invention encompass replication competent as well as replication-defective VSV vectors.

Accordingly, the present invention provides recombinant vesicular stomatitis virus (VSV) vectors comprising nucleic acid encoding a cytokine, wherein said recombinant VSV vector exhibits greater oncolytic activity against a tumor cell than a wild-type VSV vector when contacted with the tumor cell. In some examples, the cytokine is an interferon, such as interferon-beta or interferon-gamma. In other examples, the cytokine is an interleukin such as for example, IL-4 or IL-12. The present invention encompasses VSV vectors comprising nucleic acid encoding more than one biologically active protein, such as for example, a VSV vector comprising nucleic acid encoding two cytokines, such as for example, an interferon and an interleukin; two interferons; or two interleukins. In one example, a VSV vector comprises nucleic acid encoding interferon-beta and interleukin-12. A VSV vector may comprise nucleic acid encoding a heat shock protein, such as gp96 and a cytokine, such as an interferon. In other examples, the VSV vector is replication-competent. A VSV vector may comprise a human immunodeficiency virus (HIV) gp160 nucleic acid sequence, or a tumor suppressor molecule, wherein the tumor suppressor molecule is p53, mutants, variants or complementary DNA (cDNA) sequences thereof. In additional examples, the VSV vector is replication-defective. In yet other examples, the VSV vector lacks a protein function essential for replication, such as G-protein function or M and/or N protein function. The VSV vector may lack several protein functions essential for replication. In other examples, the tumor cell is a melanoma tumor cell, mammary tumor cell, prostate tumor cell, cervical tumor cell, hematological-associated tumor cell or a cell harboring a defect in a tumor suppressor pathway. The present invention also provides a replication-defective VSV vector comprising nucleic acid encoding interferon, wherein said recombinant VSV vector exhibits greater oncolytic activity against a tumor cell than a wild-type VSV vector when contacted with the tumor cell. In some examples, the interferon is interferon-beta or interferon-gamma. In other examples, the VSV vector lacks G-protein. In further examples, the tumor cell includes a melanoma tumor cell, mammary tumor cell, prostate tumor cell, cervical tumor cell, hematological-associated tumor cell or a cell harboring a defect in a tumor suppressor pathway. In further examples, an animal comprises the tumor cell and in other examples, the animal is a mammal, such as a human. The present invention also provides viral particles comprising a VSV vector of the present invention, such as a VSV vector comprising nucleic acid encoding a cytokine or suicide gene. The present invention also comprises isolated nucleic acid encoding a recombinant VSV vector of the present invention as well as host cells comprising a recombinant VSV vector of the present invention.

In a preferred embodiment, a composition comprises a vesicular stomatitis (VSV) vector encoding a human immunodeficiency virus (HIV) gp160 nucleic acid sequence, mutants, variants or complementary DNA (cDNA) sequences thereof. The VSV, in some embodiments comprises one or more foreign nucleic acid sequences for expressing one or more proteins or peptides. Examples include, without limitation, interferons, ligands, HIV or simian immunodeficiency virus (SIV) molecules, immune related molecules, cytokines, chemokines, cellular products, cell surface expression or secretion products, cell signaling molecules, or combinations thereof. Examples of immune related molecules include, without limitation: T cell receptors or ligands thereof, co-stimulatory molecules or ligands thereof, immunoglobulins, cell surface expression markers or receptors thereof. Specific examples of immune related molecules are: CD4+, CD8+, T cell receptor or ligands thereof, T cell receptor gamma or ligands thereof, NK receptors or ligands thereof, HLA class I, II and III molecules or ligands thereof, CD28, TNF receptors and ligands thereof, growth factors, interferon α-, β- and/or -γ, interleukins, growth factors, tumor necrosis factors, integrins, or combinations thereof.

The HIV or SIV nucleic acid as used herein refers to the native or mutant or variant or subunits or fragments of viral nucleic acids. For example, a full-length HIV envelope protein or mutants thereof, HIV gp160, HIV gag, HIV gp120, and full-length SIV envelope protein are some of the polypeptides that are expressed in the recombinant viral vectors of the present invention. The subunit or a fragment of the immunodeficiency envelope protein includes fragments having only a part of the contiguous amino acids of the envelope protein. These subunits or fragments include, for example, HIV gp120, HIV gp41, HIV gp40. In other embodiments the HIV protein is encoded by a gene selected from the group consisting of gag, env, pol, vif, nef, tat, vpr, rev and vpu.

In a preferred embodiment, the VSV vector further comprises one or more deletions or mutation in one or more VSV nucleic acid sequences. A wild-type VSV genome has the following gene order: 3′-NPMGL-5′. So in one embodiment, the VSV vector may lack a G protein sequence or it may have one or more mutations which result in a VSV vector lacking G-protein function or express a mutated or truncated G-protein. In another embodiment, the VSV vector has mutations or deletions of M sequences, producing VSV vectors which do not express M protein or lack M protein function or express a mutated or truncated M protein. In one embodiment, a VSV vector of the invention comprises one or more mutations in its genome. For example, a of the invention includes, but is not limited to, a VSV temperature-sensitive N gene mutation, a temperature-sensitive L gene mutation, a point mutation, a G-stem mutation, a non-cytopathic M gene mutation, a gene shuffling or rearrangement mutation, a truncated G gene mutation, an ambisense RNA mutation, a G gene insertion mutation, a gene deletion mutation and the like. Thus the term, a “mutation” includes mutations known in the art as insertions, deletions, substitutions, gene rearrangement or shuffling modifications.

In embodiments, the VSV vectors of the present invention, a polynucleotide sequence may also encode one or more heterologous (or foreign) polynucleotide sequences or open reading frames (ORFs). The foreign polynucleotide sequences can vary as desired, and include, but are not limited to, a co-factor, a cytokine (such as an interferon or interleukin) In certain embodiments, a heterologous ORF contains an HIV gene (e.g., gag, env, pol, vif, net, tat, vpr, rev or vpu). In another embodiment, the heterologous polynucleotide sequence further encodes a cytokine, such as interferon, which are selected to improve the prophylactic or therapeutic characteristics of the recombinant VSV. In preferred embodiments, a foreign nucleic acid can be inserted into regions of VSV encoding for G-protein, M-protein or combinations thereof.

In a preferred embodiment, a method of preventing or treating human immunodeficiency virus (HIV) comprises administering to an at risk patient or a patient suffering from HIV and related disorders, a therapeutically effective amount of a vesicular stomatitis (VSV) vector encoding a human immunodeficiency virus (HIV) gp160 nucleic acid sequence, mutants, variants or complementary DNA (cDNA) sequences thereof. The VSV, in some embodiments comprises one or more foreign nucleic acid sequences for expressing one or more proteins or peptides and/or further comprises one or more deletions or mutation in one or more VSV nucleic acid sequences.

In another preferred embodiment, a method of treating cancer comprising administering to a patient in need thereof, a therapeutically effective amount of a vesicular stomatitis (VSV) vector encoding a human immunodeficiency virus (HIV) gp160 nucleic acid sequence, mutants, variants or complementary DNA (cDNA) sequences thereof. The VSV, in some embodiments comprises one or more foreign nucleic acid sequences for expressing one or more proteins or peptides and/or further comprises one or more deletions or mutation in one or more VSV nucleic acid sequences. The foreign nucleic acid, e.g. gp160 can be inserted in these regions.

In another preferred embodiment, a composition comprises a vesicular stomatitis (VSV) vector expressing a tumor suppressor molecule. Preferably, the tumor suppressor molecule is p53, mutants, variants or complementary DNA (cDNA) sequences thereof.

In some embodiments, the VSV vector expressing the tumor suppressor further comprises one or more foreign nucleic acid sequences for expressing one or more proteins or peptides. Examples include interferons, ligands, immune related molecules, cytokines, chemokines, cellular products, cell surface expression or secretion products, cell signaling molecules, or combinations thereof.

In other embodiments, the VSV vector further comprises one or more deletions or mutations in one or more VSV nucleic acid sequences, e.g. G or M regions. In some embodiments, a foreign nucleic acid sequence is inserted in these regions, e.g. p53, interferons etc.

In one embodiment, a method of treating cancer comprises administering to a patient in need thereof, a therapeutically effective amount of a vesicular stomatitis (VSV) vector expressing a tumor suppressor molecule, for example p53, further comprises one or more foreign nucleic acid sequences for expressing one or more proteins or peptides. Examples include interferons, ligands, immune related molecules, cytokines, chemokines, cellular products, cell surface expression or secretion products, cell signaling molecules, or combinations thereof.

In other embodiments, a composition comprises an attenuated vesicular stomatitis (VSV) vector expressing a one or more oligonucleotides which modulate expression or function of target molecules. In embodiments, the oligonucleotides comprises: dsRNA, siRNA, antisense RNA, RNA, enzymatic RNA or microRNA.

In preferred embodiments, the oligonucleotides can be tailored to individual therapy, for example, these oligonucleotides can be sequence specific for allelic variants in individuals, the up-regulation or inhibition of a target can be manipulated in varying degrees, such as for example, 10%, 20%, 40%, 100% expression relative to the control. That is, in some patients it may be effective to increase or decrease target gene expression by 10% versus 80% in another patient.

Up-regulation or inhibition of gene expression may be quantified by measuring either the endogenous target RNA or the protein produced by translation of the target RNA. Techniques for quantifying RNA and proteins are well known to one of ordinary skill in the art. In certain preferred embodiments, gene expression is inhibited by at least 10%, preferably by at least 33%, more preferably by at least 50%, and yet more preferably by at least 80%. In particularly preferred embodiments, of the invention gene expression is inhibited by at least 90%, more preferably by at least 95%, or by at least 99% up to 100% within cells in the organism. In certain preferred embodiments, gene expression is up-regulated by at least 10%, preferably by at least 33%, more preferably by at least 50%, and yet more preferably by at least 80%. In particularly preferred embodiments, of the invention gene expression is up-regulated by at least 90%, more preferably by at least 95%, or by at least 99% up to 100% within cells in the organism.

Selection of appropriate oligonucleotides for mediating RNA interference is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of oligonucleotides that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

VSV is sensitive to the antiviral actions of the interferons (IFN). In studies with mice rendered defective in type I IFN signaling, the animals become susceptible to lethal infection by VSV. Data indicate that a functional IFN system is required to induce antiviral genes responsible for inhibiting the viral replication. One key anti-viral gene that is induced by IFN is referred to as the RNA-dependent protein kinase. (PKR). a 68 kDa serine/threonine protein kinase. That has been shown to be critical for protection against VSV infection. Down regulation of PKR protein or activity occurs in a broad spectrum of human malignancies. Therefore, tumor cells with reduced PKR activity are predicted to be more susceptible to VSV infection than their normal counterparts. WO 01/19380. Methods for measuring the activity of PKR in cells/cell lines are known in the art. Table 1 shows that interferon protects primary but not transformed cells from infection with rVSV. B16(F10) (murine melanoma), DA-3 (murine breast cancer) and HMVEC (human microvascular endothelial cells; normal cells) pretreated with IFN for 24 h were infected with WT VSV, VSV-IL-4, VSV-TK or VSV-GFP at an m.o.i. of 0.1 pfu for 18 h. Supernatants from infected cells were used to determine viral titers in plaque assays.

TABLE 1
Viral titers in interferon treated transformed and primary cells

| Cell line | Virus | PFU/ml |
|---|---|---|
| B16(F10) | WT VSV | 7.0 × 10⁵ |
| | VSV-TK | 1.0 × 10⁵ |
| | VSV-IL-4 | 6.2 × 10⁴ |
| | VSV-GFP | 4.0 × 10⁵ |
| DA-3 | WT VSV | 2.9 × 10⁷ |
| | VSV-TK | 3.1 × 10⁷ |
| | VSV-IL-4 | 2.7 × 10⁷ |
| | VSV-GFP | 3.0 × 10⁷ |
| HMVEC | WT VSV | <100 |
| | VSV-TK | <100 |
| | VSV-IL-4 | <100 |
| | VSV-GFP | <100 |

VSV replicates preferentially in malignant cells. This is primarily due to host defense mechanisms that normally contain VSV infection being damaged in cancerous cells, thus allowing the virus to propagate. The virus will destroy the malignant cells by mechanisms involving virus-induced apoptosis. Tumors grown in mice can be destroyed following intratumoral or intravenous inoculation of VSV. Table 2 provides a list of cell lines and VSV ability to replicate in these cell lines.

TABLE 2

| Cell line | Cell or tissue type | VSU infection |
|---|---|---|
| BHK | hamster kidney | + |
| HMVEC | human normal | − |
| B16 (F10) | melanoma | + |
| DA-3 | breast | + |
| MCF-7 | transformed hu breast | + |
| BC-1 | hu hematological malignancy | + |
| Jurkat | hu hematological malignancy | + |
| HL60 | hu hematological malignancy | + |
| K562 | hu hematological malignancy | + |
| PC-3 | hu transformed prostate | + |
| Hela | hu cervical tumor | + |

wherein “hu” refers to of human origin

Recombinant VSV vectors that contain suicide cassettes and/or immunomodulatory genes are shown to enhance apoptotic or antitumor immune activity. Recombinant VSV vectors have been produced that contain nucleic acid encoding the IL-4 or TL-12 gene and that express large quantities of the IL-4 or IL-12 cytokine following infection of a cell. IL-4 and IL-12 are responsible for regulating T and B-cell responses. Without being bound by theory, rVSV-IL4 or VSV-IL-12 expressing constructs should target cancer cells in the body and replicate while producing amounts of localized IL-4 or IL-12, which may stimulate cytotoxic T-cell and/or antibody responses to the tumor. This may have an amplified antitumor effect and help eradicate the malignancy. Other immunomodulatory proteins that have been inserted into VSV constructs include the interferons, chemokines, endostatin, angiostatin and heat shock protein gp96.

Recombinant VSV that contains the suicide cassette TK gene has been constructed that expresses large quantities of thymidine kinase following VSV infection of the cell. Expression of the herpes simplex virus thymidine kinase (HSV TK) in tumor cells allows the conversion of prodrugs such as ganciclovir (GCV) and acyclovir (ACV) into their monophosphate forms which are further phosphorylated by cellular kinases into their di- and triphosphate forms. The triphosphate metabolites then get incorporated into DNA and cause cell death by inhibiting mammalian DNA polymerases. Neighboring tumor cells that do not express this gene are also killed in the presence of GCV, the phenomenon known as “bystander killing”. This effect is mediated by cellular connexins and gap junctions that allow the transfer of toxic metabolites into neighboring cells. As demonstrated herein VSV vectors comprising nucleic acid encoding TK demonstrates greater killing potential to a VSV vector alone. Additional VSV constructs that comprises nucleic acid encoding other suicide genes, such as cytosine deaminase, which renders cells capable of metabolizing 5-fluorocytosine (5-FC) to the chemotherapeutic agent 5-fluorouracil (5-FU), increases cell killing and bystander effect have been produced. As will be appreciated by the skilled artisan, other suicide genes may be employed. The addition of a suicide gene to a VSV vector may improve the safety of VSV therapy for immunocompromised individuals. A VSV vector comprising nucleic acid encoding cytosine deaminase fused to uracil phosphoribosyltransferase was constructed. This VSV vector exhibited functional expression of the cytosine deaminase activity.

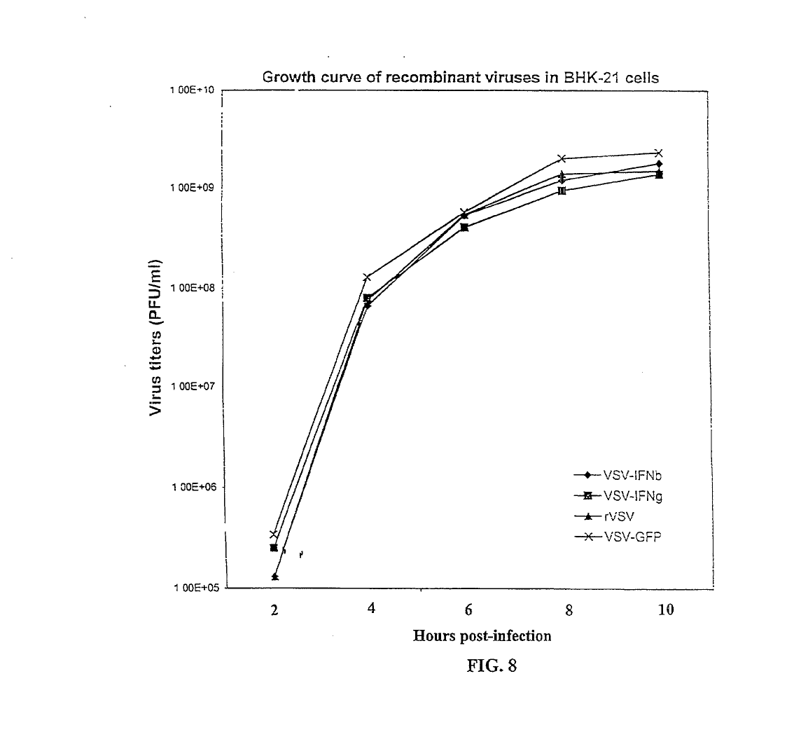

VSV vector constructs comprising nucleic acid encoding interferon, and in particular interferon-beta and interferon-gamma, have been produced. As shown in FIG. 10, high levels of functional IFN-beta are produced by cells comprising a VSV vector construct comprising nucleic acid encoding IFN-beta. As shown in FIG. 9, VSV-IFN-beta and VSV-IFN-gamma increase cell death of DA-3 cells at 24 hours after VSV infection. Additionally, recombinant VSVs efficiently produces large amounts of difficult to make/toxic/rare proteins. Thus, such viruses could be useful in making large amounts of IL-4, IL-12, IL-2 and other cytokines or other toxic or hard to make proteins. Accordingly, the invention includes VSV vectors encoding nucleic acid encoding angiostatin and endostatin, heat shock and immune costimulatory molecules have been prepared. VSV vectors and VSV viral particles can be generated to make any protein of choice, in large amounts and constitutes a eukaryotic version of the successful baculovirus/insect cell expression system. Advantages of the VSV system include high level of expression and authentic (eukaryotic) processing, unlike in insect cells.

In embodiments, VSV vector constructs comprise a recombinant vesicular stomatitis (VSV) vector encoding a human immunodeficiency virus (HIV) gp160 nucleic acid sequence, a tumor suppressor molecule, wherein the tumor suppressor molecule is p53, mutants, variants or complementary DNA (cDNA) sequences thereof.

Accordingly, the present invention provides methods for making a recombinant VSV vector of the present invention comprising growing a cell comprising said VSV vector under conditions whereby VSV is produced; and optionally isolating said VSV. In some examples, the VSV vector is replication defective and the host cells comprising the VSV protein function essential for VSV replication such that said VSV vector is capable of replication in said host cell. In some examples, the VSV vector comprises nucleic acid encoding a cytokine, such as an interferon or interleukin; a suicide gene, such as thymidine kinase or cytosine deaminase or other biological protein, such as a heat shock protein, such as for example, gp96, and endostatin and angiostatin.

Host Cells, Compositions and Kits Comprising VSV

The present invention also provides host cells comprising (i.e., transformed, transfected or infected with) the VSV vectors or particles described herein. Both prokaryotic and eukaryotic host cells, including insect cells, can be used as long as sequences requisite for maintenance in that host, such as appropriate replication origin(s), are present. For convenience, selectable markers are also provided. Host systems are known in the art and need not be described in detail herein. Prokaryotic host cells include bacterial cells, for example, E. coli., B. subtilis, and mycobacteria. Among eukaryotic host cells are yeast, insect, avian, plant, C. elegans (or nematode) and mammalian host cells. Examples of fungi (including yeast) host cells are S. cerevisiae. Kluyveromyces lactis (K. lactis), species of Candida including C. albicans and C. glabrata. Aspergillus nidulans. Schizosaccharomyces pombe (S. pombe), Pichia pastoris, and Yarrowia lipolytica. Examples of mammalian cells are COS cells, mouse L cells, LNCaP cells, Chinese hamster ovary (CHO) cells, human embryonic kidney (HEK) cells and African green monkey cells. Xenopus laevis oocytes, or other cells of amphibian origin, may also be used.

The present invention also includes compositions, including pharmaceutical compositions, containing the VSV vectors described herein. Such compositions are useful for administration in vivo, for example, when measuring the degree of transduction and/or effectiveness of oncolytic activity toward a malignant cell. Compositions can comprise a VSV vector(s) of the invention and a suitable solvent, such as a physiologically acceptable buffer. These are well known in the art. In other embodiments, these compositions further comprise a pharmaceutically acceptable excipient. These compositions, which can comprise an effective amount of a VSV vector of the invention in a pharmaceutically acceptable excipient, are suitable for systemic or local administration to individuals in unit dosage forms, sterile parenteral solutions or suspensions, sterile non-parenteral solutions or oral solutions or suspensions, oil in water or water in oil emulsions and the like. Formulations for parenteral and nonparenteral drug delivery are known in the art and are set forth in Remington's Pharmaceutical Sciences, 19th Edition, Mack Publishing (1995). Compositions also include lyophilized and/or reconstituted forms of the VSV vectors (including those packaged as a virus) of the invention.

The present invention also encompasses kits containing VSV vector(s) of this invention. These kits can be used for example for producing proteins for screening, assays and biological uses, such as treating a tumor. Procedures using these kits can be performed by clinical laboratories, experimental laboratories, medical practitioners, or private individuals.

The kits of the invention comprise a VSV vector described herein in suitable packaging. The kit may optionally provide additional components that are useful in the procedure, including, but not limited to, buffers, developing reagents, labels, reacting surfaces, means for detection, control samples, instructions, and interpretive information. The kit may include instructions for administration of a VSV vector.

Methods of Producing Recombinant VSV

The study of VSV and related negative strand viruses has been limited by the inability to perform direct genetic manipulation of the virus using recombinant DNA technology. The difficulty in generating VSV from DNA is that neither the full-length genomic nor antigenomic RNAs are infectious. The minimal infectious unit is the genomic RNA tightly bound to 1,250 subunits of the nucleocapsid (N) protein (Thomas et al., 1985, J. Virol. 54:598-607) and smaller amounts of the two virally encoded polymerase subunits, L and P. To reconstitute infectious virus from the viral RNA, it is necessary first to assemble the N protein-RNA complex that serves as the template for transcription and replication by the VSV polymerase. Although smaller negative-strand RNA segments of the influenza virus genome can be packaged into nucleocapsids in vitro, and then rescued in influenza infected cells (Enami et al., 1990, Proc. Natl. Acad. Sci. USA 87:3802-3805; Luytjes et al., 1989, Cell 59:1107-1113), systems for packaging the much larger eukaryotic genomic RNAs in vitro are not yet available.

Systems for replication and transcription of DNA-derived minigenomes or small defective RNAs from Rhabdoviruses (Conzelmann and Schnell, 1994, J. Virol. 68:713-719; Pattnaik et al., 1992, Cell 69:1011-1120) have been described. In these systems, RNAs are assembled into nucleocapsids within cells that express the viral N protein and polymerase proteins. These systems do not allow genetic manipulation of the full-length genome of infectious viruses. U.S. Pat. No. 6,168,943 discloses methods for the preparation of infectious recombinant vesiculovirus capable of replication in an animal into which the recombinant vesiculovirus is introduced. For example, U.S. Pat. No. 6,168,943 describes that vesiculoviruses are produced by providing in an appropriate host cell: (a) DNA that can be transcribed to yield (encode) vesiculovirus antigenomic (+) RNA (complementary to the vesiculovirus genome), (b) a recombinant source of vesiculovirus N protein, (c) a recombinant source of vesiculovirus P protein, and (d) a recombinant source of vesiculovirus L protein; under conditions such that the DNA is transcribed to produce the antigenomic RNA and a vesiculovirus is produced that contains genomic RNA complementary to the antigenomic RNA produced from the DNA.

Alternatively, after purification of genomic RNA, VSV mRNA can be synthesized in vitro, and cDNA prepared by standard methods, followed by insertion into cloning vectors (see, e.g., Rose and Gallione, 1981, J. Virol. 39(2):519-528). VSV or portions of VSV can be prepared using oligonucleotide restriction enzymes). Polynucleotides used for making VSV vectors of this invention may be obtained using standard methods in the art, such as chemical synthesis, recombinant methods and/or obtained from biological sources. Individual cDNA clones of VSV RNA can be joined by use of small DNA fragments covering the gene junctions, generated by use of reverse transcription and polymerase chain reaction (RT-PCR) (Mullis and Faloona, 1987, Meth. Enzymol. 155:335-350) from VSV genomic RNA (see Section 6, infra). The ability to recover fully infectious virus from a plasmid cDNA copy of the VSV genome has allowed genetic manipulation of this virus to become feasible.

In an example disclosed herein, a cDNA clone representing the entire 11,161 nucleotides of VSV has been generated and unique Xho 1/Nhe1 sites were added to facilitate entry of a heterologous gene, e.g. for example. HSV-TK. Transcription of the cDNA is dependent on T7 RNA polymerase. Vaccinia vTF7-3 was used to infect baby hamster kidney cells (BHK-21), to provide a source of polymerase. Subsequently, VSV cDNA was transfected into the same cells together with three other plasmids that express the VSV N, P and L proteins. These latter three proteins facilitate the assembly of nascent VSV antigenomic RNA into nucleocapsids and initiate the VSV infectious cycle. After 24 hours, host cells were lysed, clarified and residual vaccinia removed by filtration through a 0.2 um filter onto fresh BHK cells. Only recombinant VSVs are produced by this method since no wild-type VSV can be generated (Rose et al., 1995, P.N.A.S. USA).

VSV may be genetically modified in order to alter it properties for use in vivo. Methods for the genetic modification of VSV are well established within the art. For example, a reverse genetic system has been established for VSV (Roberts et al., Virology, 1998, 247: 1-6) allowing for modifications of the genetic properties of the VSV. Standard techniques well known to one of skill in the art may be used to genetically modify VSV and introduce desired genes within the VSV genome to produce recombinant VSVs (see for example, Sambrooke et al., 1989, A Laboratory Manual, New York: Cold Spring Harbor Laboratory Press. For insertion of nucleotide sequences into VSV vectors, for example nucleotide sequences encoding a cytokine, or for VSV gene sequences inserted into vectors, such as for the production helper cell lines, specific initiation signals are required for efficient translation of inserted protein coding sequences. These signals include the ATG initiation codon and adjacent sequences. In cases where an entire VSV gene, such as G-protein including its own initiation codon and adjacent sequences are inserted into the appropriate vectors, no additional translational control signals may be needed. However, in cases where only a portion of the gene sequence is inserted, exogenous translational control signals, including the ATG initiation codon, must be provided. The initiation codon must furthermore be in phase with the reading frame of the protein coding sequences to ensure translation of the entire insert. These exogenous translational control signals and initiation codons can be of a variety of origins, both natural and synthetic.

Following infection of a host cell, recombinant VSV shuts down host cell protein synthesis and expresses not only its own five gene products, but also heterologous proteins encoded within its genome. Successful expression of heterologous nucleic acid from VSV recombinants requires only the addition of the heterologous nucleic acid sequence into the full-length cDNA along with the minimal conserved sequence found at each VSV gene junction. This sequence consists of the polyadenylation/transcription stop signal (3′ AUACU7) followed by an intergenic dinucleotide (GA or CA) and a transcription start sequence (3′-UUGUCNNUAG) complementary to the 5′ ends of all VSV mRNAs. Ball et al. 1999, J. Virol. 73:4705-4712; Lawson et al. 1995, P.N.A.S. USA 92:4477-4481; Whelan et al. 1995, P.N.A.S. USA 92:8388-8392. Additionally, restriction sites, preferably unique, (e.g., in a polylinker) are introduced into the VSV cDNA, for example in intergenic regions, to facilitate insertion of heterologous nucleic acid, such as nucleic acid encoding an interleukin or interferon. In other examples, the VSV cDNA is constructed so as to have a promoter operatively linked thereto. The promoter should be capable of initiating transcription of the cDNA in an animal or insect cell in which it is desired to produce the recombinant VSV vector. Promoters which may be used include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78: 1441-1445), the regulatory sequences of the metallothionein gene (Brinster et al., 1982, Nature 296:39-42); heat shock promoters (e.g., hsp70 for use in Drosophila S2 cells); the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol Cell Biol. 5:1639-1648; Hammer et al. 1987, Science 235:53-58; alpha 1-antitrypsin gene control region which is active in the liver (Kelsey et al., 1987, Genes and Devel. 161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); and myosin light chain-2 gene control region which is active in skeletal muscle (Sani, 1985, Nature 314:283-286). Preferably, the promoter is an MA polymerase promoter, preferably a bacteriophage or viral or insect RNA polymerase promoter, including but not limited to the promoters for T7 RNA polymerase, SP6 RNA polymerase, and T3 RNA polymerase. If an RNA polymerase promoter is used in which the RNA is not endogenously produced by the host cell in which it is desired to produce the recombinant VSV, a recombinant source of the RNA polymerase must also be provided in the host cell. Such RNA polymerase are known in the art.

The VSV cDNA can be operably linked to a promoter before or after insertion of nucleic acid encoding a heterologous protein, such as a mammalian protein including a cytokine or a suicide gene. In some examples, a transcriptional terminator is situated downstream of the VSV cDNA. In other examples, a DNA sequence that can be transcribed to produce a ribozyme sequence is situated at the immediate 3′ end of the VSV cDNA, prior to the transcriptional termination signal, so that upon transcription a self-cleaving ribozyme sequence is produced at the 3′ end of the antigenomic RNA, which ribozyme sequence will autolytically cleave (after a U) this fusion transcript to release the exact 3′ end of the VSV antigenomic (+)RNA. Any ribozyme sequence known in the art may be used, as long as the correct sequence is recognized and cleaved. (It is noted that hammerhead ribozyme is probably not suitable for use.)

VSV vectors of the present invention comprise one or more heterologous nucleic acid sequence(s) encoding a mammalian protein, such as for example, a cytokine, suicide gene or heat shock protein gp96, or a reporter gene, such as for example, green fluorescent protein, or a tumor suppressor molecule, wherein the tumor suppressor molecule is p53, mutants, variants or complementary DNA (cDNA) sequences thereof. Examples of cytokines include, but are not limited to interferons (IFN), including IFN-beta, IFN-gamma, INF-alpha. INF-omega, and INF-epsilon; tumor necrosis factor (TNF), lymphotoxin, interleukins (IL), including but not limited to IL-2, IL-4, and IL-12 and granulocyte macrophage colony-stimulating factor (GM-CSF). A VSV vector may comprise nucleic acid encoding two cytokines, such as for example, two interleukins, two interferons or an interleukin and an interferon. VSV vectors of the present invention, comprise one or more heterologous nucleic acids encoding a viral molecule, such as for example, a human immunodeficiency virus (HIV) gp160 nucleic acid sequence mutants, variants or complementary DNA (cDNA) sequences thereof.

The sequences for most of the genes encoding IFN as they occur in nature are published and many have been deposited with the American Type Culture Collection (ATCC) (Rockville, Md.). A VSV vector of the present invention can encode any form of IFN. In some examples, the nucleic acid encodes a human form of IFN. This includes human IFN, IFN-alpha, IFN-beta, IFN-gamma, IFN-omega, and IFN-epsilon. Human INF-alpha sequences are described in Weber et al. (1987, EMBO. J 6:591-598); human IFN-beta sequences are described in U.S. Pat. No. 5,908,626 and Fiers et al. (1982) Philos. Trans. R. Soc. Land., B, Biol. Sci. 299:29-38) and has been deposited with GenBank under Accession No. M25460; human INF-gamma is available from the ATCC and has ATCC accession numbers 39047 and 39046 and a particular form of recombinant human IFN-gamma is commercially available (rhIFN-gamma-1b, ACTIMMUNE™, Genentech, Inc. South San Francisco, Calif.); and human IFN-epsilon sequence are disclosed in U.S. Pat. No. 6,329,175. The human IL-2 gene has been cloned and sequenced and can be obtained as, for example, a 0.68 kB BamHI-HinDIII fragment from pBC12/HIV/IL-2 (available from the American Type Culture Collection (“ATCC”) under Accession No. 67618). U.S. Pat. No. 5,951,973 discloses the sequence for mouse and human 1L-4. Interleukin-12 (IL-12), originally called natural killer cell stimulatory factor, is a heterodimeric cytokine described, for example, in M. Kobayashi et al, J. Exp. Med, 170:827 (1989). The expression and isolation of IL-12 protein in recombinant host cells is described in detail in International Patent Application WO90/05147, published May 17, 1990 (also European patent application No. 441,900). The DNA and amino acid sequences of the 30 kd and 40 kd subunits of the heterodimeric human IL-12 are provided in the above recited international application. Research quantities of recombinant human and murine IL-12 are also available from Genetics Institute, Inc., Cambridge, Mass. Further, the sequences of human GM-CSF, human TNF and human lymphotoxin are known and are available. The sequence of human GM-CSF is known (Wong et al. (1985) Science 228:810-815) and has been deposited with GenBank under Accession No. M 10663. The sequence of human TNF has been described (Wang et al. (1985) Science 228: 149-154) and is 5 deposited with GenBank under Accession No. M 10988. The sequence of human lymphotoxin (TNF-13) has also been published (Iris et al. (1993) Nature Genet. 3:137-145) and is deposited with GenBank under Accession No. Z15026.

A VSV vector or viral particle of the present invention can comprise nucleic acid encoding a suicide gene, such as thymidine kinase (TK), such as herpes simplex TK or cytosine deaminase (CD), such as for example E. coli CD. The HSV-TK gene has been previously mapped, cloned and sequenced, and is readily available (EMBL HEHSVLTK, Accession X03764, EMBL HEHS07, Accession V00466). The HSV-tk gene can be obtained from natural sources, such as from the viral genome of Herpes simplex virus type I (HSV-1) or from the 15 Herpes simplex virus type II (HSV-2) genome. The varicella zoster virus (VZV) genome also includes a specific thymidine kinase gene (VZV-tk) which has been cloned, sequenced and characterized (Mori et al. (1988) Intervirology 29:301-310, (1986) J. Gen. Virol. 67:1759-1816). Thus, the VZV-tk gene can be obtained from the VZV genome. The E. coli cytosine deaminase gene has also been cloned and sequenced (Danielson et al. (1992) Mol. Microbiol. 6:1335-1344, Austin et al. (1993) Mol. Pharmacol. 43:380-387, Dong et al. (1996) Human Gene Therapy 7:713-720), and the gene sequence has been deposited with GenBank under Accession No. S56903. The E. coli cytosine deaminase gene can therefore also be obtained from a number of natural or synthetic sources known to those skilled in the art. Alternatively, cytokine or suicide gene oligonucleotides can be synthetically derived, using a combination of solid phase direct oligonucleotide synthesis chemistry and enzymatic ligation methods which are conventional in the art. Synthetic sequences can be prepared using commercially available oligonucleotide synthesis devices such as those devices available from Applied Biosystems, Inc. (Foster City, Calif.).

The present invention encompasses expression systems comprising a VSV vector comprising one or more heterologous nucleotide sequence(s), such as, a nucleotide sequence encoding a cytokine, such as for example, interferon, or two cytokines, or a suicide gene, such as for example, TK, inserted within a region of the VSV essential for replication, such as the G glycoprotein region, or other region essential for replication, such that the VSV lacks the essential function and is replication-defective. The VSV vector may have a mutation, such as a point mutation or deletion of part or all, of any region of the VSV genome, including the G, M, N, L or P region. If the mutation is in a region essential for replication, the VSV will be grown in a helper cell line that provides the essential region function. The VSV may also comprise a mutation, such as for example, a point mutation or deletion of part or all of a nucleotide sequence essential for replication, and optionally, with the heterologous nucleotide sequence inserted in the site of the deleted nucleotide sequence. The heterologous nucleotide sequence may be operably linked to a transcriptional regulatory sequence. Following infection of a target malignant or tumor cell, progeny viruses will lack essential protein function and cannot disseminate to infect surrounding tissue. In additional embodiments, the VSV vector is mutated in nucleic acid, such as by point mutation, substitution or addition of nucleic acid, or deletion of part or all, of nucleic acid encoding other VSV protein function such as, M protein and/or N protein function. VSV may be targeted to a desired site in vitro to increase viral efficiency. For example, modification of VSV G protein (or other VSV proteins) to produce fusion proteins that target specific sites may be used to enhance VSV efficiency in vivo. Such fusion proteins may comprise, for example, but not limited to single chain Fv fragments that have specificity for tumor antigens. (Lorimer et al., P.N.A.S. U.S.A., 1996. 93: 14815-20).

A VSV vector lacking a gene(s) essential for viral replication can be grown in an appropriate complementary cell line. Accordingly, the present invention provides recombinant helper cell lines or helper cells that provide a VSV protein function essential for replication of a replication-deficient VSV construct. In some examples, the protein function is G-protein function. For example, a VSV vector comprising nucleic acid encoding a cytokine and lacking G-protein function can be grown in a cell line, i.e., a helper cell line, for example, a mammalian cells line such as CHO cell line, permissive for VSV replication, wherein said cell line expresses an appropriate G-protein function, such that said VSV is capable of replicating in the cell line. These complementing or helper cell lines are capable of allowing a replication-defective VSV to replicate and express one or more foreign genes or fragments thereof encoded by the heterologous nucleotide sequence. In some embodiments, the VSV vector lacks a protein host cell line comprises nucleic acid encoding the protein function essential for replication, such as for example, VSV G-protein function. Complementing cell lines can provide VSV viral function through, for example, co-infection with a helper virus, or by integration or otherwise maintaining in stable form part or all of a viral genome encoding a particular viral function. In other examples, additional VSV non-essential proteins can be deleted or heterologous nucleotide sequences inserted into nucleotide regions encoding non-essential VSV, such as for example, the M and N proteins. The heterologous nucleotide sequence can be inserted into a region non-essential for replication wherein the VSV is replication competent. Heterologous nucleotide sequences can be inserted in non-essential regions of the VSV genome, without necessitating the use of a helper cell line for growth of the VSV vector.

The recombinant VSV of the invention are produced for example, by providing in an appropriate host cell VSV cDNA wherein said cDNA comprises nucleotide sequence encoding a heterologous protein, such as for example, a cytokine, including interleukin or interferon, or a suicide gene. The nucleic acid encoding a heterologous protein can be inserted in a region non-essential for replication, or a region essential for replication, in which case the VSV is grown in the presence of an appropriate helper cell line. In some examples, the production of recombinant VSV vector is in vitro. in cell culture, in cells permissive for growth of the VSV. Standard recombinant techniques can be used to construct expression vectors containing DNA encoding VSV proteins. Expression of such proteins may be controlled by any promoter/enhancer element known in the art. Promoters which may be used to control expression of VSV proteins can be constitutive or inducible.

The host cell used for recombinant VSV production can be any cell in which VSV grows, e.g., mammalian cells and some insect (e.g., Drosophila) cells. Primary cells lacking a functional INF system, or in other examples, immortalized or tumor cell lines can be used. A vast number of cell lines commonly known in the art are available for use. By way of example, such cell lines include but are not limited to BHK (baby hamster kidney) cells, CHO (Chinese hamster ovary) cells, HeLA (human) cells, mouse L cells, Vero (monkey) cells, ESK-4, PK-15, EMSK cells, MDCK (Madin-Darby canine kidney) cells, MDBK (Madin-Darby bovine kidney) cells, 293 (human) cells, and Hep-2 cells. Such cell lines are publicly available for example, from the ATCC and other culture depositories.

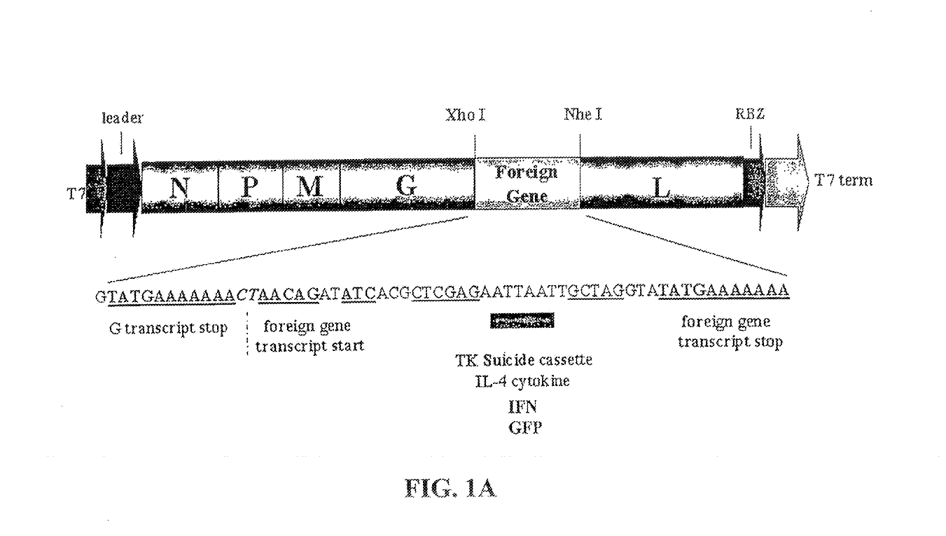

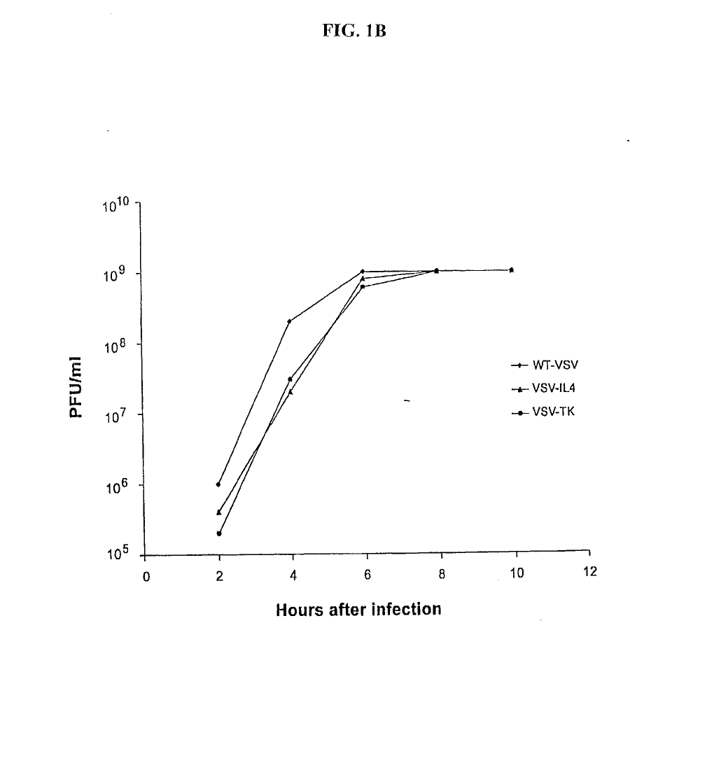

In examples disclosed herein, the plasmid, pVSV-XN2 was constructed as shown in FIG. 1A. The genes encoding HSV-TK, mouse IL-4, INF-beta or INF-gamma or GFP were cloned into the pVSV-XN2, between the VSV G and L genes. Recombinant VSV (rVSV) produced by cell lines can be isolated using for example, an affinity matrix. Method of isolating VSV by affinity matrix are described in for example, WO 01/19380. Briefly, methods for isolating a rVSV from comprising adding the VSV to an affinity matrix, to produce bound VSV, washing the bound VSV, and eluting the VSV from the affinity matrix. The present invention encompasses a modified VSV that comprises a non-naturally occurring fusion protein on the outer surface of the virus. The non-native protein may be a fusion protein comprising an affinity tag and a viral envelope protein or it may be derived from a producer cell. Producer cell lines may be engineered to express one or more affinity tags on their plasma membranes which would be acquired by the virus as it buds through the membrane. One example of an affinity tag is the use of Histidine residues which bind to immobilized nickel columns. Affinity tags also include antibodies. Other protocols for affinity purification may be used as known within the art, for example, but not limited to, batch processing, a solution of virus and affinity matrix, pelleting the VSV-bound matrix by centrifugation, and isolating the virus. Alternatively, VSV can be collected and purified as described in U.S. Pat. No. 6,168,943. Briefly, VSV is collected from culture supernatants, and the supernatants clarified to remove cellular debris. One method of isolating and concentrating the virus is by passage of the supernatant through a tangential flow membrane concentration. The harvest can be further reduced in volume by pelleting through a glycerol cushion and by concentration on a sucrose step gradient.

Methods of Using Recombinant VSV Vectors of the Invention

The subject VSV vectors and viral particles can be used for a wide variety of purposes, which will vary with the desired or intended result. Accordingly, the present invention includes methods using the VSV vectors described herein.

The invention provides methods for producing oncolytic activity in a tumor cell and/or malignant cell and/or cancerous cell comprising contacting the cell, including, for example, a tumor cell or a malignant cell in metastatic disease, with a VSV vector of the invention, wherein said VSV vector exhibits greater oncolytic activity against the cell than a wild-type VSV vector. In some examples, the contacting is effected by administration, such as for example, intravenous injection to an individual comprising said cell. In other examples, the contacting is effected by administration, such as by intratumoral injection to an individual comprising said cell. For these methods, the VSV vector may or may not be used in conjunction with other treatment modalities for producing oncolytic activity, such as, for example, tumor suppression, including but not limited to chemotherapeutic agents known in the art, radiation and/or antibodies. The invention also provides compositions comprising a VSV vector comprising nucleic acid encoding a cytokine or a suicide gene wherein said VSV vector is present in the composition in an amount effective to produce oncolytic activity when said composition is administered to the tumor and/or malignant cells. In some examples, the composition further comprises a pharmaceutical excipient. In other examples, the composition is administered intratumorally or intravenously.

Accordingly, the present invention provides methods for producing oncolytic activity in a tumor cell comprising the step of contacting the cell with a recombinant VSV vector comprising nucleic acid encoding a cytokine, wherein said VSV vector exhibits greater oncolytic activity against the tumor cell than a wild-type VSV vector. In some examples of the methods, the VSV vector is replication-defective. In other examples, the VSV vector lacks G-protein function. In yet further examples, the cytokine is an interferon, such as for example, interferon-beta or interferon-gamma; or a cytokine, such as for example, an interleukin, such as interleukin-4 or interleukin-12. In other examples, a recombinant vesicular stomatitis (VSV) vector encodes a human immunodeficiency virus (HIV) gp160 nucleic acid sequence, a tumor suppressor molecule, wherein the tumor suppressor molecule is p53, mutants, variants or complementary DNA (cDNA) sequences thereof. In additional examples, the tumor cell includes a melanoma tumor cell, mammary tumor cell, prostate tumor cell, cervical tumor cell, hematological-associated tumor cell or cell harboring defects in a tumor suppressor pathway. In yet further examples, said contacting is by intravenous injection to an individual comprising said tumor cell or by intratumoral injection to an individual comprising said tumor cell.

The present invention also provides methods for producing oncolytic activity in a tumor cell, comprising the step of contacting the tumor cell with a recombinant VSV vector comprising nucleic acid encoding a suicide gene wherein said VSV vector exhibits greater oncolytic activity against the tumor cell when administered along with a prodrug than a wild-type VSV vector. In some examples of the methods, the suicide gene encodes thymidine kinase (TK) and the prodrug is ganclyclovir or acyclovir. In other examples, the suicide gene encodes a cytosine deaminase and the prodrug is 5-fluorocytosine. In some examples of the methods, the VSV vector is replication-defective. In other examples, the VSV vector lacks G-protein function. In yet other examples of the methods, the tumor cell includes melanoma tumor cell, mammary tumor cell, prostate tumor cell, cervical tumor cell, hematological-associated tumor cell or cell harboring a defect in a tumor suppressor pathway. In other examples, the contacting is by intravenous injection to an individual comprising said tumor cell or by intratumoral injection to an individual comprising said tumor cell.

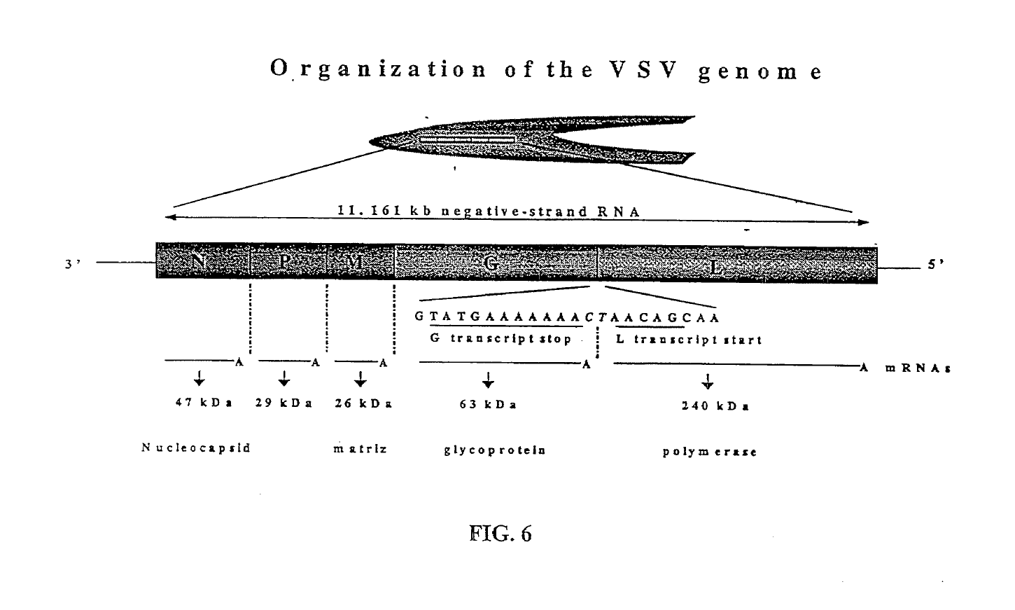

The invention also provides methods of treatment, in which an effective amount of a VSV vector(s) described herein, or a composition comprising a VSV vector of the present invention described herein, is administered to an individual comprising, having or suspected of having a malignant cell and/or tumor cell and/or cancerous cell. VSV was shown to induce cell death in transformed human cell lines including those derived from breast (MCF7), prostate (PC-3), or cervical tumors (HeLa), as well as a variety of cells derived from hematological-associated malignancies (HL 60, K562, Jurkat, BC-1). BC-1 is positive for human herpesvirus-8 (HHV-8), overexpresses Bcl-2 and is largely resistant to a wide variety of apoptotic stimuli and chemotherapeutic strategies. The results of additional studies indicated that VSV could induce apoptosis of cells specifically transformed with either Myc or activated Ras and transformed cells carrying Myc or activated Ras or lacking p53 or overexpressing Bcl-2 are susceptible to VSV replication and viral-induced apoptosis. FIGS. 7 A-7B illustrate that several human cancer cell lines are permissive to VSV replication and lysis. Therefore, it is predicted that administration of a VSV vector of the present invention or a composition comprising a VSV vector of the present invention would produce oncolytic activity in a variety of malignant cells or tumor cells. Methods for screening cells or cell lines, including malignant cells lines, for susceptibility to infection with a VSV vector of the present invention, can be performed by methods disclosed in WO 01/19380. Briefly a method for identifying a tumor 20 susceptible to treatment with a virus, comprises: (a) dividing a sample containing cells of the tumor into a first portion and a second portion; (b) treating portion with the VSV virus; and (c) determining whether the percentage of dead cells in the first portion is higher than in the second portion, wherein the tumor is susceptible to treatment with the VSV virus if the percentage of dead cells in the first portion is higher than in the second portion.

The present invention encompasses treatment using a VSV vector(s) in individuals with malignant cells and/or tumor cells susceptible to VSV infection, as described above. Also indicated are individuals who are considered to be at risk for developing tumor or malignant cells such as those who have had previous disease comprising malignant cells or tumor cells or those who have had a family history of such tumor cells or malignant cells. Determination of suitability of administering VSV vector(s) of the invention will depend on assessable clinical parameters such as serological indications and histological examination of cell, tissue or tumor biopsies. Generally, a composition comprising a VSV vector(s) in a pharmaceutically acceptable excipient is administered.

Accordingly, the present invention provides methods for suppressing tumor growth, comprising the step of contacting the tumor with a recombinant VSV vector comprising nucleic acid encoding a cytokine, wherein said VSV vector exhibits greater tumor suppression than a wild-type VSV vector. In some examples, a recombinant vesicular stomatitis (VSV) vector encodes a tumor suppressor molecule, wherein the tumor suppressor molecule is p53, mutants, variants or complementary DNA (cDNA) sequences thereof. In some examples of the methods, the VSV vector is replication defective. In other examples, the VSV vector lacks G-protein function. In yet further examples, the cytokine is an interferon, such as for example, interferon-beta or interferon-gamma; or a cytokine, such as for example, an interleukin, such as interleukin-4 or interleukin-12. The present invention also provides methods for suppressing tumor growth, comprising the step of contacting the tumor with a recombinant VSV vector comprising nucleic acid encoding a suicides gene wherein said VSV vector exhibits greater tumor suppression when administered along with a prodrug than a wild-type VSV vector. In some examples of the methods, the VSV vector is replication-defective. In other examples, the VSV vector lacks G-protein function. In yet further examples, the suicide gene is thymidine kinase and the prodrug is ganclyclovir or acyclovir. In other examples, the suicide gene encodes a cytosine deaminase and the prodrug is 5-fluorocytosine. In yet other examples of the methods, the tumor cell includes melanoma tumor cell, mammary tumor cell, prostate tumor cell, cervical tumor cell, hematological-associated tumor cell or cell harboring a defect in a tumor suppressor pathway.

The present invention encompasses ex vivo treatment of cells or tissues using a VSV vector(s) in individuals with malignant cells and/or tumor cells and/or cancerous cells susceptible to VSV infection, as described above. Ex vivo treatment of cells can be undertaken in an attempt to reduce or eliminate undesirable malignant or cancerous cells from a mixture of cells. Such cells include for example, bone marrow cells or peripheral blood stem cells. Accordingly, the present invention provides a method for the ex-vivo treatment of cells whereby a cell population from an individual comprising undesirable cells or suspected of comprising undesirable cells is contacted with a VSV vector of the present invention, such as a VSV vector comprising nucleic acid encoding a cytokine or a suicide gene. After the contacting, the cell population may be transplanted back into the individual. Ex vivo purging of cells using viruses is described in for example, WO 02/00233.

The present invention also encompasses the use of VSV vectors, including VSV vectors comprising nucleic acid encoding one or more cytokine(s) such as, for example an IFN, including IFN-beta and IFN-gamma, to infect tumor and/or malignant and/or cancerous cells, in vitro. The present invention encompasses any tumor and/or malignant and/or cancerous cell that is sensitive to VSV infection. The VSV vector replicates in the tumor cells, expressing the cytokine (immunomodulatory gene) to high levels. The cells are inoculated into an animal, (in some examples, back into the animal from which they were obtained) which makes an immune response to the infected, lysed tumor cells. The VSV expressed cytokines stimulate the host's immune response. Thus, an individual, such as a mammal, including a human, could be protected from subsequent tumor challenge, if exposed to the tumor and/or malignant and/or cancerous cells that have been contacted with a VSV vector of the present invention and subsequently lysed, thereby creating a “cancer vaccine” effect. The use of VSV vectors of the present invention as “cancer vaccines” can be tested in an animal model by obtaining tumors grown in a mouse; contacting the tumors with a VSV vector of the present invention, such as a VSV vector comprising nucleic acid encoding a cytokine, such as for example, an IFN, IL-12 and/or heat shock protein, in vitro. Then different mice (same strain) that have the same tumor type growing in them are inoculated with the VSV-infected tumor cells. The VSV infected tumor cells lyse in the animal, attract the host's immune system and eradicate the established tumor (post-vaccine). In contrast, lysed tumor cells not exposed to virus are poor immunogens. The use of VSV virus infection attracts an immune response in the animal. In some examples, the VSV vector is replication-defective. In other examples, the VSV vector is replication-competent. Accordingly, the present invention provides compositions capable of eliciting an immune response in an individual comprising tumor cells infected with or lysed by a VSV vector of the present invention. The present invention also provides methods for eliciting an immune response to tumor cells in an individual comprising administering a composition comprising tumor cells infected with or lysed by a VSV vector of the present invention to said individual. In some examples, the VSV vector comprises nucleic acid encoding one or more cytokines, such as an interferon or interleukin. In other examples, the VSV vector comprises nucleic acid encoding a immunomodulatory protein, such as a chemokine, or a heat shock protein, such as gp96, under conditions such as, for example, gp96. The present invention also provides methods for protecting an individual against tumor challenge comprising, contacting tumor cells derived from an individual with a VSV vector comprising nucleic acid encoding a cytokine, an immunomodulatory protein or a heat shock protein, such as gp96, under conditions suitable for lysing said tumor cells; and returning said lysed tumor cells to said individual.

Once a VSV vector comprising a nucleotide sequence encoding a cytokine or suicide gene has been obtained, the VSV vector, or VSV particles comprising the vector, can be administered to an individual in need. Such an individual can comprise malignant cells or tumor cells or can be at risk for developing malignant cells or tumor cells or development of metastatic disease. The VSV constructs of the present invention comprising a cytokine or suicide cassette can be used to treat local tumors or metastatic disease. A variety of cells and cells lines, including ovarian carcinoma cells, fibrosarcoma, lung carcinoma, melanoma, prostate carcinoma, lung carcinoma and leukemia cells are sensitive to VSV infection. Therefore, such tumor cells and/or malignant cells derived therefrom may be particularly amenable to treatment with a VSV expressing a cytokine or a suicide gene. As disclosed herein, VSV expressing cytokines or suicide genes have been shown to exhibit greater oncolytic activity than wt VSV. It is expected that VSV will have oncolytic activity when administered locally to the tumor cells or malignant cells, that is intratumorally, as well as when administered distal to the tumor or malignant cell, such as via intravenous administration or by other routes.

To evaluate whether genetically engineered VSV carrying immunomodulatory or suicide genes, such as thymidine kinase, can be created and whether such viruses are more efficacious in tumor therapy than the wild type VSV, VSV vectors carrying the herpes virus thymidine kinase suicide cassette (TK) or the cytokine gene interleukin-4 (IL-4) were developed. It is known that the mechanism of TK action involves the TK protein phosphorylating the nontoxic pro-drug ganciclovir (GCV), which becomes incorporated into cellular DNA during replication leading to chain tem1ination and cell death. The TK/GCV suicide approach has been reported to have additional benefits in that modified TK can be directly passed from the transduced cell to adjacent cells thereby increasing tumor killing, a phenomenon known as the “bystander effect”. The activities of IL-4, in contrast, involve influencing the development of effector cells such as eosinophils and antigen presenting cells. IL-4 is also known to regulate T helper (Th) cell development into Th2 cells and assist in the stimulation of a humoral response (Asnagli et al. 2001, Curr. Opin. Immunol. 13:242-7). High levels of IL-4 have been reported to be critical for the rejection of tumors in the initial phases of tumor development and implanted engineered IL-4 secreting cells as well as viral vectors transducing IL-4 have been shown to mediate the regression of a number of malignancies including melanoma, glioma and colon carcinoma (Benedetti et al. 2000, Nat. Med. 6:447-50; Giezeman-Smits, 2000, Cancer Res 60:2449-50; Nagai, et al. 2000, Breast Cancer 7: 181-6; Tepper et al. 1992, Science 257:548-51).

Results from experiments disclosed herein demonstrate that a VSV vector comprising nucleic acid encoding TK exhibits oncolytic activity against systemic 25 and sub-cutaneous tumors and stimulates anti-tumor T-cell response. Data also demonstrate that VSV-IL4 or VSV-TK induce apoptosis, in vivo, of highly aggressive melanoma cells when an animal is infected at an m.o.i. of 1 or less. The data also demonstrate that VSV-TK and VSV-IL4 exhibit oncolytic activity superior to VSV alone in examples disclosed herein.

VSV vectors comprising nucleic acid encoding interferon. in particular, interferon-beta and interferon-gamma, were developed. Results from experiments described herein demonstrate that a VSV vector comprising nucleic acid encoding IFN-beta or IFN-gamma replicates in malignant cells and kills them. The data also demonstrate that VSV-INF-beta and INF-gamma exhibit oncolytic activity superior to VSV alone.

Methods of Administration

Many methods may be used to administer or introduce the VSV vectors or viral particles into individuals, including but not limited to, oral, intradermal, intramuscular, intraperitoneal, intravenous, intratumor, subcutaneous, and intranasal routes. The individual to which a VSV vector or viral particle is administered is a primate, or in other examples, a mammal, or in other examples, a human, but can also be a non-human mammal including but not limited to cows, horses, sheep, pigs, fowl, cats, dogs, hamsters, mice and rats. In the use of a VSV vector or viral particles, the individual can be any animal in which a VSV vector or virus is capable of growing and/or replicating. The present invention encompasses compositions comprising VSV vector or viral particles wherein said compositions can further comprise a pharmaceutically acceptable carrier. The amount of VSV vector(s) to be administered will depend on several factors, such as route of administration, the condition of the individual, the degree of aggressiveness of the malignancy, and the particular VSV vector employed., Also, the VSV vector may be used in conjunction with other treatment modalities.

If administered as a VSV virus, from about 10² up to about 10⁷ p.f.u., in other examples, from about 10³ up to about 10⁶ p.f.u., and in other examples, from about 10⁴ up to about 10⁵ p.f.u. can be administered. If administered as a polynucleotide construct (i.e., not packaged as a virus), about 0.01 μg to about 100 μg of a VSV construct of the present invention can be administered, in other examples, 0.1 μg to about 500 μg, and in other examples, about 0.5 μg to about 200 μg can be administered. More than one VSV vector can be administered, either simultaneously or sequentially. Administrations are typically given periodically, while monitoring any response. Administration can be given, for example, intratumorally, intravenously or intraperitoneally.

Pharmaceutically acceptable carriers are well known in the art and include but are not limited to saline, buffered saline, dextrose, water, glycerol, sterile isotonic aqueous buffer, and combinations thereof. One example of such an acceptable carrier is a physiologically balanced culture medium containing one or more stabilizing agents such as stabilized, hydrolyzed proteins, lactose, etc. The carrier is preferably sterile. The formulation should suit the mode of administration.

The composition, if desired, can also contain minor amounts of wetting or emulsifying agents or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration.

In a specific embodiment, a lyophilized recombinant VSV of the invention is provided in a first container; a second container comprises diluent consisting of an aqueous solution of 50% glycerin, 0.25% phenol, and an antiseptic (e.g., 0.005% brilliant green).

The precise dose of VSV vector or viral particles to be employed in the formulation will also depend on the route of administration, and the nature of the patient, and should be decided according to the judgment of the practitioner and each patient's circumstances according to standard clinical techniques. The exact amount of VSV vector or virus utilized in a given preparation is not critical provided that the minimum amount of virus necessary to produce oncolytic activity is given. A dosage range of as little as about 10 mg, up to amount a milligram or more. is contemplated.

Effective doses of the VSV vector or viral particle of the invention may also be extrapolated from dose-response curves derived from animal model test systems. A table of safety of recombinant viruses on BALB/c mice is shown below.

TABLE 3
Table Safety of recombinant viruses on BALB/c mice

| | | Amount of virus | N. of dead | Mortality |
|---|---|---|---|---|
| Inoculation | Virus | (PFU) | mouse | (%) |
| I.V. | VSV-GFP | 1 × 10\hat{ }6 | 0/5 | 0 |
| | | 5 × 10\hat{ }6 | 0/5 | 0 |
| | | 2 × 10\hat{ }7 | 0/5 | 0 |
| | | 5 × 10\hat{ }7 | 0/5 | 0 |
| | | 1 × 10\hat{ }8 | 5/5 | 100 |
| | VSV-IFNβ | 1 × 10\hat{ }6 | 0/6 | 0 |
| | | 5 × 10\hat{ }6 | 0/6 | 0 |
| | | 2 × 10\hat{ }7 | 0/6 | 0 |
| | | 5 × 10\hat{ }7 | 0/5 | 0 |
| | | 1 × 10\hat{ }8 | 2/5 | 40 |
| | VSV-IFNγ | 1 × 10\hat{ }6 | 0/6 | 0 |
| | | 5 × 10\hat{ }6 | 0/6 | 0 |
| | | 2 × 10\hat{ }7 | 0/6 | 0 |
| | | 5 × 10\hat{ }7 | 4/5 | 80 |
| | rVSV | 1 × 10\hat{ }6 | 0/5 | 0 |
| | | 5 × 10\hat{ }6 | 0/5 | 0 |
| | VSV-IL12 | 2 × 10\hat{ }7 | 0/2 | 0 |
| | | 5 × 10\hat{ }7 | 2/2 | 100 |
| | | 1 × 10\hat{ }8 | 4/4 | 100 |

The data show that the growth of VSV-IFN-beta is attenuated compared to VSV -GFP. The following examples are offered by way of illustration and should not be considered as limiting the scope of the invention.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

### Example 1

### Materials and Methods

Experimental Protocol

Cell lines.

BHK-21 and B16(F10) melanoma cells were obtained from American Type Culture Condition (ATCC, Manassas, Va.). BHK cells were grown in Dulbecco's Modified Essential Medium (DMEM) containing 10% fetal bovine serum (FBS, Hyclone Laboratories Inc, Logan, Utah). B16(F10) cells were propagated in similar medium except that it contained low sodium bicarbonate (1.5 g/L). D1-DMBA3 breast tumor and DA-3 cells derived from the tumor were a gift from Dr. Diana Lopez (University of Miami, Miami, Fla.). Human primary cells (microvascular endothelial-HMVEC) were obtained from Clonetics Corp (San Diego, Calif.) and grown according to the manufacturer's specifications.

Construction of Recombinant Viruses.

The IL-4, TK and GFP inserts were amplified from pGexp, pKO-TK and pLEGFP-Cl (Clontech Laboratories, Palo Alto, Calif.) plasmids respectively by PCR. For IL-4, the primers 5′ GGCACTCGAGATGGGTCTCAACCCCCAGCTAGTTG and 5′ GCCGTCTAGACTACGAGTAATCCATTTGCATGATGC were used.

For the GFP gene, the primers used were

5′ GGCACTCGAGATGGTGAGCAAGGGCGAGGAG
and
5′ GCTTGAGCTCTA GATCTGAGTCCCTACTTGTACAGC.

The TK gene was amplified using the following primers

5′ CTTGTAGACTCGA GTA TGGCTTCGT ACCCCGGCCATCAG
5′ GTATTGTCTGCTA GCGTGTTTCAGTT AGCCTCCCCCATC.

The IL-4 and GFP PCR products were digested with Xhol and Xhal while the TK PCR product was digested with Xhol and Nhel and ligated to pVSV-XN2 (a gift from Dr. John Rose, Yale University) that had been digested with Xho1 and Nhe1 (compatible with Xba1). The plasmid pVSV-XN2 contains the entire VSV genome and has unique Xho1 and Nhe1 sites flanked by VSV transcription start and stop signals. The procedure for recovering infectious recombinant VSV viruses was similar to that described previously (Lawson et al. 1995, P.N.A.S. USA 92:4477-81; Whelan et al., 1995 P.N.A.S. USA 92:8388-92).

In Vitro Cell Killing Assay.

Murine B16, DA-3 and human HMVECs cells were seeded in 12 well plates at approximately 2×10⁵ cells/well. Cells were pretreated with interferon (human interferon-α-2a [Hoffman-La Roche, Nutley N.J.] for HMVECs, mouse interferon-αβ [Sigma, St Louis, Mich.] for B16 and DA-3) at 1000 u/ml for 24 hours. The cells were then infected with wt VSV, VSV-TK, VSV-IL-4 or VSV-GFP at an m.o.i. of 0.1 for 18 hours, trypsinized and counted by Trypan Blue exclusion analysis.

Enzyme-Linked Immunosorbent Assay for IL-4 Production.

Levels of IL-4 in the rVSV-IL-4 supernatants obtained 24 hours following infection of BHK cells with rVSV-IL-4 were determined by using an IL-4 ELISA kit obtained from Pharmingen (San Diego, Calif.) following the manufacturer's protocol.

Thymidine Kinase Enzyme Assay.

Phosphorylation of ganciclovir was used to measure functional levels of HSV-TK in extracts of VSV-TK infected cells as follows (Yamamoto et al. 1997, Cancer Gene Ther 4:91-6). After a 24 15 hour infection with wild type or recombinant viruses, BHK cells were washed twice with PBS and resuspended in a buffer containing 0.5% NP-40, 50 mM Tris HCl (pH 7.5), 20% glycerol, 5 mM benzamidine, 2 mM dithiothreotol and 0.5 mM PMSF. They were subjected to four cycles of freeze thawing followed by centrifugation of the lysates at 13,000 rpm for 5 mM. at 4° C. The enzyme assay was carried out using 60 μg protein in a reaction mix containing 50 mM Tris HCl, 5 mM magnesium chloride, 5 mM ATP, 10 mM sodium fluoride and 2 mM dithiothreotol in a 37° C. water bath. 20 μl aliquots were taken at 0, 30 and 60 minutes following addition of [³H] GCV (45 μM) and spotted on DE-81 (Whatman) paper. The filter papers were washed twice in 1 mM ammonium formate and extracted with 0.1 M KCl/0.1 N HCl and counted in a scintillation counter.

Tumor Inoculation in Mice.

Female C5781/6 or Balb/c mice (8 week old) from Jackson laboratories were inoculated subcutaneously with 5×10⁵B16(F10) melanoma cells (left flank) or 1.5×10⁶ breast tumor cells (mammary pad). Mice were divided into four groups of five each according to the type of virus administered-heat inactivated VSV, wild type VSV virus, VSV-TK or VSV-IL-4. After the development of palpable tumors the mice received 2×10⁷ pfu of the wild type or recombinant VSV viruses intratumorally followed by a second injection three days later. Mice that received VSV-TK were also administered GCV (100 mg/kg body weight) one day following the initial injection, followed by daily injections for 10 days thereafter. Tumors were measured three times weekly. Mice were sacrificed once tumors reached greater than 1.8 cm in any diameter. Mean tumor volumes in the four groups were compared using one way ANOVA analysis. Histopathology was carried out as described previously (Balachandran et al. 2001, J. Virol. 75:3474-9).

Cytotoxic Assays.

Single cell spleen suspensions were cultured in 25 mm upright flasks at a 20:1 ratio with mitomycin C treated B16F10 cells for 3 days in the presence of 400 pg/ml IL-2 (Calbiochem, San Diego, Calif.) at 37° C. in a humidified 5% C02 atmosphere. The cytotoxic activity of spleen cells was determined by performing a standard chromium release assay using 0.1 mCi of Na₂¹⁵CrO₄ (Amersham, Arlington Heights, Ill.) at 37° C., 2 h. After 4 h incubation of effector and target cells, the supernatants were harvested using a SKATRON cell harvester and the amount of ⁵¹ Cr release determined in a gamma counter (Beckman, Palo Alto, Calif.). The percent specific lysis was calculated by the following equation: experimental release CPM-spontaneous release CPM/maximum release CPM-spontaneous release CPM X 100. Maximum release was the cpm obtained by incubating target cells with 2% SDS (Fisher), and spontaneous release was determined by incubation with growth medium alone. Spontaneous release of ⁵¹ Cr was always less than 15% of the total release in these assays.

### Example 2

### Generation of rVSV Expressing TK or IL-4

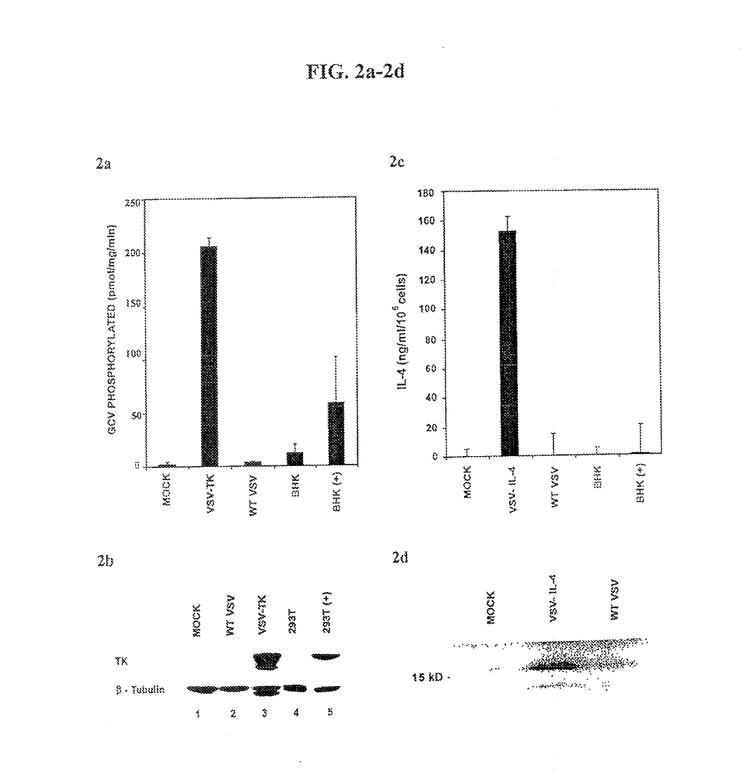

To evaluate whether VSV could be generated to express potential anticancer genes, the HSV-TK, mouse IL-4 or, control green fluorescent protein (GFP) were cloned into the plasmid pVSV-XN2 as additional transcription units between the VSV G and L genes (FIG. 1a). Recombinant VSVs expressing either TK, IL-4 or GFP from the modified transcription unit were recovered in cells expressing the full-length antigenomic VSV RNA containing the additional gene as well as the VSV nucleocapsid (N), phosphoprotein (P) and polymerase (L) proteins. Preliminary analysis indicated that viable recombinant viruses could be obtained in all cases and examination of virus production per cell, by one-step growth curve studies, indicated no aberrant variation in replication abilities compared to the wild-type VSV (FIG. 1b). Indeed, all viruses grew to exceptionally high titers of approximately 10⁹ plaque forming units (pfu) per mi. We next determined whether the recovered VSVs expressed TK, IL-4, or GFP. Accordingly, BHK cells were infected with VSV-TK for 24 hours at a multiplicity of infection (m.o.i.) of 1 and infected cells were examined for TK protein expression by immunoblot analysis using an anti-TK monoclonal antibody. Results indicated that the TK protein was being synthesized to extremely high levels in cells infected with VSV-TK, while in contrast we were unable to detect any TK being expressed in cells infected with other types of VSV (FIG. 2b lanes 1-3). Confirmation of TK expression was demonstrated using immunofluorescent microscopy of VSV-TK infected cells while BHK cells infected with VSV-green fluorescent protein (GFP) also expressed high levels of GFP as determined by fluorescent microscopy. To ascertain whether the TK was functional, GCV phosphorylation levels were measured in cells infected at an m.o.i. of 1 with VSV-TK or wild-type virus, 8 hours post-infection. The results indicated that TK phosphorylated GCV at high levels, on average approaching 200 pmoles/mg/min, which we estimated to be nearly 60-fold greater than in wildtype VSV infected cells (FIG. 2a). The data disclosed herein thus indicate that VSV can be generated to express high levels of functional TK, without any adverse effects upon virus replication.

We next analyzed whether VSV could express functional IL-4. Since IL-4 is rapidly secreted from the cell following translation, preliminary immunoblot analysis of VSV-IL-4 infected cell extracts using an anti-murine IL-4 monoclonal antibody indicated very little IL-4 present in the cytoplasm of infected cells, as expected. However, capture enzyme-linked immunoabsorbant assay (ELISA) using a monoclonal antibody that binds functional IL-4 indicated that the cytokine was being secreted into the culture medium at very high levels from VSV-IL-4 infected cells (FIG. 2c). Indeed, VSV-IL-4 generated about 150 ng/ml of IL-4 per 10⁶ cells, over a hundred fold greater than in the same number of BHK cells transfected with IL-4 cDNA under control of the CMV promoter. Confirmation of IL-4 expression, was obtained by immunoprecipitating secreted IL-4 from the culture medium of ³⁵S labeled VSV-IL-4 infected cells using an anti-IL-4 monoclonal antibody (FIG. 2d). Thus, similar to our observations characterizing VSV-TK, we demonstrate that VSV can also be engineered to express high levels of the cytokine IL-4, which is biologically active.

### Example 3

### rVSV Expressing TK or IL-4 Retain Oncolytic Activity

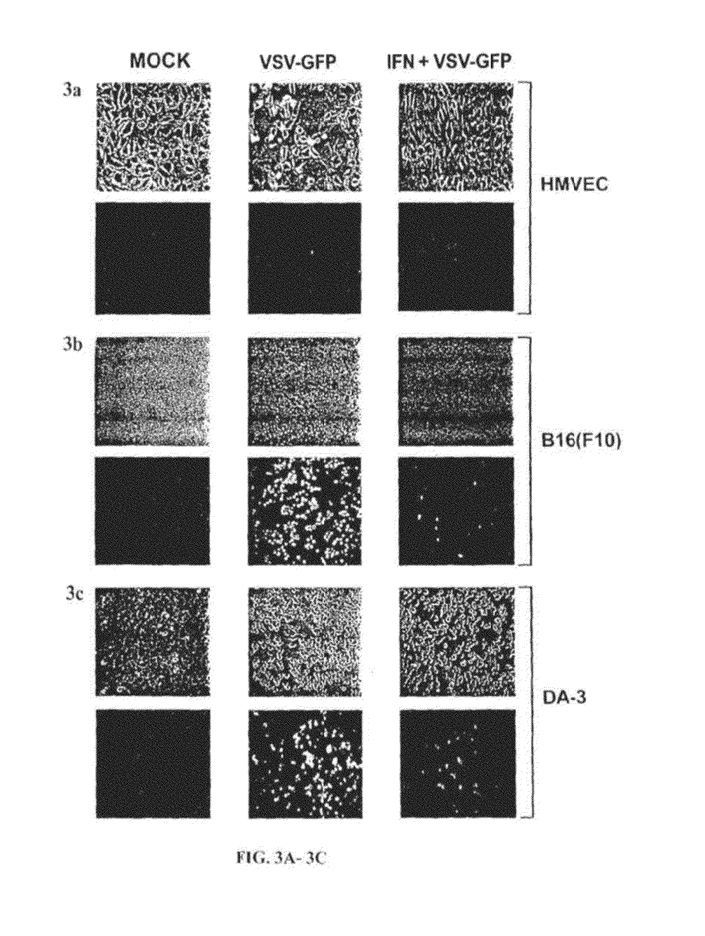

To evaluate whether the recombinant viruses expressing IL-4 or TK retained their ability to preferentially replicate in malignant cells, to eventually induce cell death, a number of transformed cells were examined in infection assays. An important objective was also to compare the effects of VSV and recombinant VSVs on normal cells. To start to appraise this, we selected human microvascular endothelial cells (HMVECs) since they would be most likely to be exposed to VSV infection after subcutaneous (s.c.) or intravenous (i.v.) administration in tumor therapy. HMVECs (10⁶) were therefore infected at an m.o.i of 0.1 for 18 hours with wild-type VSV or VSV-IL-4, VSV-TK or VSV-GFP. Cell viability was measured using Trypan Blue exclusion analysis and revealed that approximately 20-30% of the HMVECs underwent cell death, an effect that could be essentially eliminated following pre-treatment with interferon (IFN-α) [FIGS. 3a and d]. In contrast, a similarly infected murine breast tumor cell-line (Sotomayor, et al. 1991, J Immunol. 14 7:2816-23) (DA-3, derived from D1 DMBA-3 tumor) as well as a melanoma cell-line B 16(F10) (Fidler et al., 1975, Cancer Res. 35, 218-24) underwent dramatic cytolysis (80-90%) following infection with either the wild-type virus, VSV-TK, VSV-IL-4 or VSV-GFP. VSV-TK induced potent cytolysis of cells even in the absence of GCV. Pretreatment of B16(F10) cells with IFN (1000 u/ml for 24 hours) reduced the amount of virus-mediated cell death observed following infection, regardless of the virus used. It remained plausible that the mechanisms of IFN product ton may be predominantly defective in B16(F10) cells in view of the fact that IFN-signaling to induce antiviral protection appears partially intact. However, subsequent analysis of viral production in IFN pre-treated B16(F10) cells revealed relatively high virus replication (10⁵/ml), suggesting incomplete protection and anti-viral activity (Table 1). In contrast, Trypan Blue exclusion analysis indicated that IFN did not afford any significant protection of breast tumor derived DA-3 cells, suggesting that IFN function may be defective at multiple levels in these cells (FIGS. 3c and f). These data were confirmed by determining that virus replication in IFN-treated DA-3 cells was exceedingly high (10⁷/ml) compared to control HMVECs in which virus production was almost completed ablated (Table 1). To evaluate the mechanisms of virus-mediated cell lysis, infected B6(F10) and DA-3 cells were evaluated for apoptotic activity 18 hours post-infection. The mechanisms of cytolysis invoked by recombinant VSV-IL-4 or TK as well as the wild-type virus involved the induction of apoptosis since levels of active caspase-8 and 9 was three-fold higher than in untreated cells. Thus, recombinant VSV expressing IL-4 or TK do not appear to lose their effectiveness at inducing programmed cell death in infected cells compared to wild-type VSV.

### Example 4

### rVSV Expressing TK or IL-4 Kill Tumors In Vivo

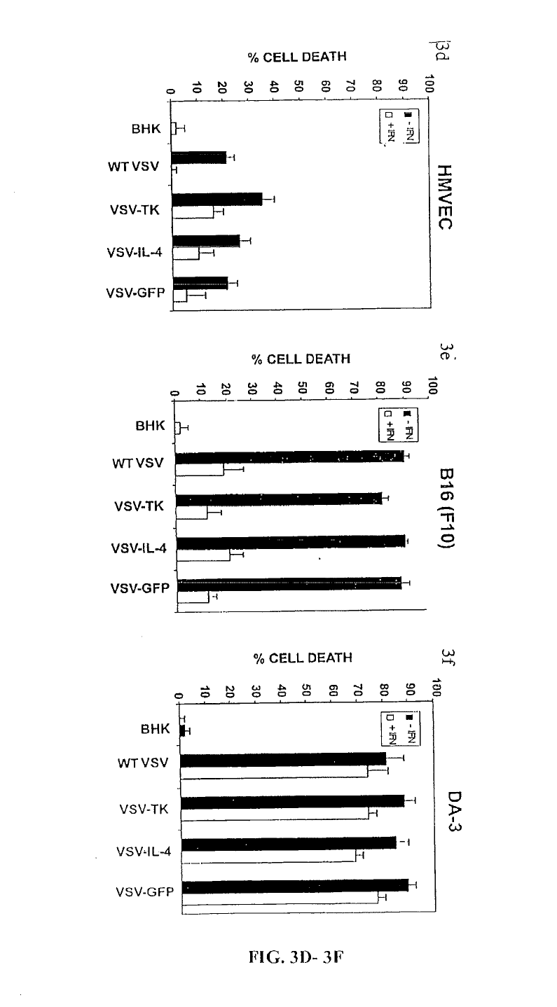

To evaluate the oncolytic activity of the recombinant viruses, immunocompetent mice were sub-cutaneously (s.c.) implanted with 1×10⁶ cells of the syngeneic B16 melanoma (C57B1/6) or poorly immunogenic mammary tumor derived D1 DMBA (Balb/c), both aggressive tumors. Following the formation of palpable tumors, 2×10⁷ wild-type VSV or VSV-IL-4 or VSV-TK were introduced intratumorally (i.t.). As controls, an equivalent amount of heat-inactivated (HI) VSV was used. Ganciclovir was administered (100 mg/kg body weight), intraperitoneally (i.p.), daily in animals receiving VSV-TK. Virus therapy was repeated once more, three days after the first injection and tumor growth monitored three times weekly. Resultant data demonstrated that wild-type VSV inhibited the growth of both the melanoma and breast tumors compared to tumors treated with control HI virus (FIGS. 4a and h). However, in independent sets of experiments, more potent inhibition of tumor growth (both B 16 and D-1 DMBA) was observed in animals treated with either VSV-IL-4 or VSV-TK (FIGS. 4a and h). In some instances, complete regression of tumors was observed in animals implanted with either B16(F10) (3/5 mice) or D1 DMBA (2/5 mice), following treatment with VSV-TK. In contrast, a number of mice implanted with either tumor and infected with HI-VSV had to be sacrificed 4 days post-treatment because of the excessive tumor size. The differences in the tumor volume between the control group (HI-VSV) and animals treated with either VSV-TK or VSV-IL-4 was observed to be statistically significant at (<0.01) and (p<0.001) for animals implanted with B16(F10) or D1 DMBA, respectively. These data indicate that VSV expressing either IL-4 or TK exhibit potent oncolytic activity, superior to that of VSV alone.

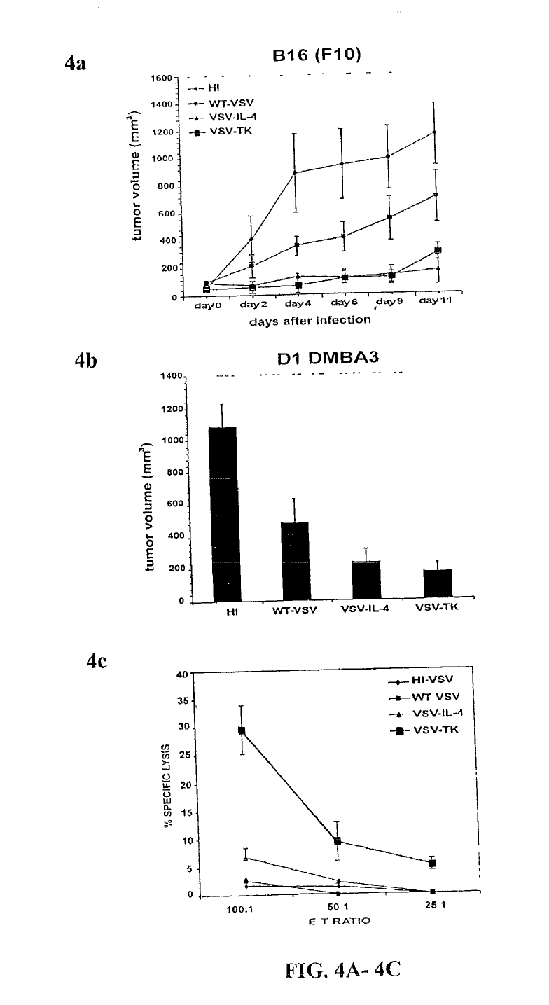

To examine the effects of virus therapy, in vivo, tumors inoculated with the various viruses were excised and sections examined histologically following hematoxylin and eosin (H&E) staining. As expected, tumors infected with control HI-VSV exhibited very little morphological abnormalities that could be associated with virus induced oncolytic activity (<30% necrosis [FIG. 5a]). However, a greater proportion of cell death, as exhibited by pyknotic nuclei, was observed in B16(F10) tumors treated with wild-type VSV (˜50%) or with VSV-IL-4 (75%) or VSV-TK (˜95%), indicative of an increase in oncolytic activity (FIG. 5b-d).

Significant inflammatory infiltration was also evident in tumors treated with the recombinant viruses, especially in tumors treated with VSV-IL-4, which showed major infiltration of polymorphonuclear cells including neutrophils and eosinophils (FIG. 5 compare e to g). Analysis of viral replication in the brain, liver and tumors retrieved from mice implanted with B16(F10) or D1 DMBA cells and treated with wild-type or recombinant viruses (two were analyzed from each virus treated group) did not reveal evidence of infectious virus 7 days after the last virus treatment (one week after the primary inoculation). Thus, following i.t. inoculation, all VSV types appear to be rapidly cleared from the animals. Although CD8+ T-lymphocytes have been reported to be important for the antitumor activity of IL-4, cellular immune responses have also been reported to play a role in the local and systemic antitumor activity of TK/GCV (Yamamoto et al. 1997, Cancer Gene Ther 4:91-6). Since IL-4 and TK/GCV treated tumors exhibited pronounced host cell infiltration, we examined whether lymphocytes generated by the animals exhibited specific cytotoxic activity to tumor associated 5 antigens in the implanted syngeneic transformed cells. Accordingly, animals harboring B16(F10) derived tumors were intratumorally inoculated twice with wild-type VSV, HI VSV, VSV-IL-4 or VSV-TK. Seven days after inoculation, spleens were removed, mononuclear cells isolated and chromium release assays carried out using B16(F10) target cells. The results indicated that animals generated a robust CTL-response only against tumors receiving VSV-TK, and not with VSV-IL-4, the wild-type virus or controls. The data are consistent with previous findings that TK/GCV-mediated destruction of tumor cells can facilitate antigen uptake by professional antigen presenting cells. Possibly, the process of cellular destruction involves apoptosis through accumulation of p53 and the upregulation of Fas (CD-95), which then through aggregation stimulates FADD-dependent cell death in a Fas ligand independent manner (Beltinger et al. 1999, P.N.A.S. USA 96:8699-704). It is therefore plausible that VSV expressing TK exerts a greater oncolytic effect through TK/GCV mediated apoptosis and enhanced bystander effect, as well as through the generation of specific antitumor CTL responses. While IL-4 can influence the development of Th cells, IL-4 in this tumor model did not strongly influence the development of tumor specific cytotoxic T cells, as judged by CTL assays. Nevertheless, VSV-expressing IL-4 did exert a greater oncolytic effect that was statistically significant compared to wild-type VSV.

Although IL-4 has been incorporated into a number of live virus vectors for either gene therapy, anticancer strategies or to increase an immune response to candidate viral-vaccines, the overall positive effects of the cytokines contribution vary (Bebedetti et al. 2000, Nat. Med. 6:447-50). The data shown herein indicate that tumors treated with VSV expressing IL-4 may exert a more potent oncolytic effect than VSV alone possibly due to the increased presence of infiltrating eosinophils and neutrophils, which have been reported to directly have antitumor activity (Tepper et al., 1992, Science 257:548-51). However, the full mechanisms of IL-4 mediated antitumor activity undoubtedly remain to be clarified.

A recent report indicated that expression of IL-4 by ectromelia virus suppressed antiviral cell-mediated immune responses and was associated with high mortality in mice usually resistant to the wild-type virus (Jackson et al., 2001, J. Virol. 75: 1205). While these data raise concerns about developing novel viruses that contain immunomodulatory genes, the data would be consistent with studies where retroviruses or adenovirus expressing IL-4 produced no adverse effects in vivo (Steele et al., 2000, Proc. Soc. Exp. Biol. Med. 223:118-27). In safety trials we did not note any increase in toxicity in animals inoculated, by various routes, with VSV-IL-4 compared to the wild-type VSV. Aside from not being able to detect infectious VSV in organs or tumors from mice receiving VSV treatment, 7 days after the last tumor inoculation, animals receiving VSV-IL-4 or VSV-TK at 2×10⁷ (i.p.) or 2×10⁶ (i.v.), presently remain healthy 8 weeks after infection.

### Example 5

### Materials and Methods

Cells:

BHK-21 cells, primary and transformed mouse embryonic fibroblasts derived from C57BL/6 mice were maintained in Dulbecco's modified essential medium (DMEM) supplemented with 10% fetal bovine serum (HyClone Laboratories Inc., Logan, Utah), 100 units of penicillin G/ml, 100 units of streptomycin/ml and 0.25 pg of amphotericin B. B6(F10) melanoma cells and DA-3 cells derived from D1-DMBA3 breast tumor were maintained in same medium except that it contained 1.5 g of sodium bicarbonate per liter and OP1 media supplement (Sigma, St. Louis, Mo.), respectively. TS/A mammary adenocarcinoma cells were maintained RPM 11640 medium supplemented with 10% fetal bovine serum.

Construction of Recombinant Virus:

Mouse IFN-β cDNA was amplified by polymerase chain reaction (PCR) from plasmid pMK-Mβ, a gift from Dr. Yoichiro Iwakura, Institute of Medical Science, University of Tokyo, using oligonucleotides 5′-TCCATCCTCGAGCACTATGAACAACAGGTGGATCCTC-3′ (sense) and 5′AGGTCTGCTAGCCTAGTTTTGGAAGTTTCTGGT-3′ (anti-sense). The amplified fragment was then inserted into the Xho 1-Nhe I site of pVSV-XN2 (Fernandez et al. 2002, J. Vivol.; 76(2): 895-904). The procedure for recovering recombinant VSV was similar to that described previously (Lawson et al., 1995, P.N.A.S. USA, 92:4477-81; Whelan et al., 1995, P.N.A.S. USA. 92:8388-92). The seed virus was propagated in BHK-21 cells and stored at −80 C until use. VSV-GFP and rVSV were prepared as previously described (Fernandez et al., 2002).

Virus Growth In Vitro:

The growth of the recombinant viruses in bhk-21 cells were examined as previously described (Fernandez et al., 2002). Cells were seeded in 6-well culture plate at 1×10⁶ cells per well and infected with each virus at a multiplicity of infection (m.o.i.) of 10 PFU per cell. The culture supernatants were harvested at the indicated times and subjected to titer determination by a standard plaque assay on BHK-21 cells.

For in vitro cell-killing assay, murine embryonic fibroblasts, B16(F10), DA-3, and TS/A cells were seeded in 12- or 24-well culture plate at approximately 2×10⁵ cells per well and infected with viruses at the indicated m.o.i. for 24 h, and then trypsinized and counted by trypan blue exclusion analysis.

ELISA for IFN-β Production:

Expression levels of IFN-β on VSV infected cells was examined by an enzyme-linked immunosorbent assay (ELISA). BHK-21 cells were seeded in a 35-mm-diameter culture dish at 1×10⁶ cells and infected with viruses at an m.o.i of 10 p.f.u. per cell for 24 h. The supernatant was then subjected to ELISA. ELISA was performed as previously published procedure (Coligan et al., 1991). Briefly, 96-well microplate (Nalge Nunc International, Rochester, N.Y.) was coated with 5 μg/ml of monoclonal rat antibody to mouse IFN-β (Seikagaku America, Falmouth, Mass.) and incubated overnight at 4 C. Serial dilutions of the supernatants were then added and incubated overnight at 4 C. Polyclonal sheep antibody to mouse IFN-α/β (United States Biological, Swampscott, Mass.) diluted to 1:2000 was used as secondary antibody, and bound antibodies were detected with peroxidase-labeled polyclonal rabbit antibody to sheep IgG (H+L) (KPL, Gaithersburg, Md.) diluted to 1:2500. The peroxidase was revealed by incubation with the substrate 2,2′-azino-bis(3-ethylbenzothiazoline-6-sulfonate) for 30 min, and a spectrophotometric reading was obtained at 414 nm

Biological Activity Assay for IFN-β:

BHK-21 cells were seeded in a 35-mm-diameter culture dish at 1×10⁶ cells and infected with viruses at an m.o.i of 10 p.f.u. per cell for 24 h. The supernatants were harvested and treated at 56 C for 30 min to inactivate the viral infectivity. B16(F10) cells were seeded in a 24-well culture plate at 2×10⁶ cells per well and incubated with the supernatant diluted to 1:50 or 500 units/ml of mouse IFN-α/β (Sigma) for 24 h. Cells were then infected with VSV at an m.o.i. of 0.1 for 24 h, and CPE was assessed under microscopy.

Animal Studies:

Six to 8-week-old female BALB/c mice were obtained from Jackson Laboratories and maintained under specific pathogen-free conditions. Mice were injected intravenously (i.v.) with 5×10⁴ pfu TS/A cells and then infected i.v. with 5×10⁷ p.f.u. of recombinant VSV 2 days later. The survival of mice was monitored daily after virus infection. For vaccination of VSV-IFNβ-infected TS/A cells, the cells were infected with the virus at an m.o.i. of 10 per cell for 2 h. Mice were injected subcutaneously (s.c.) with 1×10⁶ infected TS/A cells and challenged sub-cutaneously with 1×10⁵ TS/A cells 10 days later. The tumor sizes were measured at 2 days intervals, and the volume was calculated according to the formula 0.5× length×(width²).

Statistical significance of inter-group differences was evaluated using the Mann-Whitney test. For histopathological analysis, mice were killed at indicated time point, and tumors were excised, fixed with 10% neutralized buffered formalin and stained with hematoxylin and eosin.

Cytotoxic Assay:

Cytotoxicity of T-lymphocytes was performed as described as previously (Fernandez et al., 2002). Briefly, spleen cells prepared from VSV-infected tumor-bearing mice were incubated with ⁵¹Cr-labeled TS/A cells at indicated effecter cells to target cells ratios, and release of ⁵¹Cr was quantified on a gamma counter. The percentage of lysis was calculated according to the formula [(experimental release cpm spontaneous release cpm)/(maximum release−spontaneous release)]×100.

Table 4 shows the virus titers in primary and transformed C57BL/6 mouse fibroblast. Table 5 shows the virus titers in TS/A cells.

TABLE 4
Virus titers in primary and transformed C57BL/6 mouse fibroblasts

Virus titers (PFU/1 × 10\hat{ }5 cells)^{b)}

| Virus^{a)} | Primary Cell | Transformed Cell |
|---|---|---|
| VSV-GFP | 1.3 × 10\hat{ }7 | 3.6 × 10\hat{ }6 |
| VSV-IFNβ | 6.2 × 10\hat{ }4 | 2.9 × 10\hat{ }5 |
| VSV-IFNγ | 3.5 × 10\hat{ }6 | 1.5 × 10\hat{ }6 |

^{a)}MOI 0.01
^{b)}Data shows a mean of twice of independent experiment

TABLE 5
Virus titers in TS/A cells

Virus titers (PFU/1 × 10\hat{ }6 cells)^{a)}

| Virus | M.O.I. 0.1 | M.O.I. 0.01 |
|---|---|---|
| VSV-GFP | 4.3 × 10\hat{ }8 | 1.4 × 10\hat{ }8 |
| VSV-IFNβ | 3.5 × 10\hat{ }8 | 1.4 × 10\hat{ }7 |
| VSV-IFNγ | 3.4 × 10\hat{ }8 | 1.3 × 10\hat{ }8 |

^{a)}Data shows a mean of twice of independent experiment

The data show that VSV-IFN-beta or IFN-gamma retain the ability to kill tumor cells. The inclusion of the IFN-beta or IFN-gamma in the VSV vector construct does not impede the VSV oncolytic activity or replicative abilities.

### Example 6

### VSV Inhibits Growth Ofp53-Deficient, Myc-Transformed, or Ras-Transformed In Vivo

To start to evaluate the use of VSV in antitumor therapy, athymic nude mice were subcutaneously implanted with 2×10⁶ C6 glioma cells, or with Balb-3T3 cells transformed with the Myc or the activated K-Ras gene. When palpable tumors had formed (approximately 7-14 days when the tumors had reached an approximate size of 0.25 cm²) mice were infected intratumorally with VSV (2.5×10⁷ pfu/ml) and monitored daily. The injection, with the same amount of virus was repeated after four days. All mice that received VSV showed markedly-inhibited tumor growth, irrespective of the genetic backgrounds of the tumors, or the oncogenic events contributing to their transformation. In fact, the administration of VSV resulted in marked repression of tumor growth in all animals tested within 17 days, when tumors in the control animals exceeded that acceptable tumor burden. These data highlight the potent efficacy of VSV against tumors both in vitro and in vivo.

To examine whether VSV spread beyond the implanted, virus-inoculated tumor, a variety of tissue from the VSV treated animals, as well as the tumors themselves were analyzed for the presence of residual, replicating VSV. Examination of VSV infected mice for the presence of VSV 21 days after infection revealed the existence of residual virus (2×10⁴−3.5×10⁵ pfu/g) in tumor tissue derived from C6 cells. However, no virus was detectable in the lung, brain, kidney, spleen, or liver of mice receiving VSV therapy after this period of time. These data show that VSV replication is restricted to tumor-lineage.

### Example 7

### VSV Exerts Anti-Tumor Activity Intravenously and on Distal Tumors

To examine whether VSV was capable of exerting its antitumor effects following inoculation at sites distant from the tumor, VSV was introduced intravenously (i.v., three injections of 1×10⁷ pfu each/mouse two days apart) and monitored growth of implanted C6 glioblastoma tumors every day for up to 8 days.

Nude mice were implanted with 1×10⁶ C6 cells bilaterally into both rear flanks of the mouse and the right tumor was inoculated with 1×10⁷ pfu VSV, or with heat-inactivated control virus. Nude mice bearing single C6 tumors were injected intravenously with 1×10⁸ pfu VSV at days 1, 3, 5, 7, 9, 11 and 13. i.v.-inoculated VSV was able to cause the regression of C6 tumors in vivo.

Next, experiments were designed to determine whether intratumoral inoculation of VSV can cause the regression of distal tumors at other sites on the mouse. For these experiments, nude mice were implanted with C6 glioblastoma cells bilaterally on both the left and right flanks of the mouse and inoculated only one of the implanted tumors with VSV after both had reached a size of approximately 0.25 cm². VSV, but not heat-inactivated virus control, was able to cause the partial regression of distal tumors when introduced into one tumor. These studies demonstrate the potential of VSV to eradicate tumors and metastases at sites distal from the site of inoculation.

### Example 8

### VSV from cDNA

A cDNA clone representing the entire 11,161 nucleotides of VSV was generated and unique Xho 1/Nhe I sites were added to facilitate entry of a heterologous gene, in our case, HSV-TK. Transcription of the cDNA is dependent on T7 RNA polymerase. Vaccinia vTF7-3 is used to infect baby hamster kidney cells (BHK-21), to provide a source of polymerase. Subsequently, VSV cDNA is transfected into the same cells together with three other plasmids that express the VSV N, P and L proteins. These latter three proteins facilitate the assembly of nascent VSV antigenomic RNA into nucleocapsids and initiate the VSV infectious cycle. After 24 hours, host cells are lysed, clarified and residual vaccinia removed by filtration through a 0.2 urn filter onto fresh BHK cells. Only recombinant VSVs are produced by this method since no wild-type VSV can be generated.

### Example 9

### Characterization of Recombinant VSVs (rVSVs)

Cells infected with rVSV or wild-type VSV are metabolically labeled with [³⁵S]methionine. Cells are lysed and aliquots analyzed by SDS-PAGE. Since VSV inhibits host proteins synthesis, only viral proteins are made, including heterologous genes inserted into its genome. Cells infected with rVSVs will have an extra protein (i.e. HSV-TK, ˜26 kDa) being synthesized compared to control cells infected with VSV alone. VSV mRNAs are detected by a similar manner exists. Therefore, ELISAs are used to detect the expression of heterologous proteins, such as, IL-4, IL-12 and IFNs. High levels of heterologous protein expression have been obtained in all recombinant systems examined

### Example 10

### Growth of VSV

Large amounts of VSV (Indiana strain) and recombinant VSV are purified by sucrose gradients. Essentially, BHK cells are infected at 0.01 m.o.i and after 24 hours, where >80% of cells usually exhibit CPE/apoptosis, supernatants are collected and clarified by centrifugation. Clarified supernatants are purified by centrifugation through 10% sucrose and the viral pellets resuspended and layered onto continuous 35-55% sucrose gradients. The gradients are centrifuged at 110,000 g for 18 hours at 4° C. and virus retrieved and pelleted by further centrifugation and 15,000 rpm at 4° C. for 1 hour. Viruses are resuspended in PBS, concentrations determined by standard plaque assays and stored in aliquots at −80° C. (30).

### Example 11

### Generation of Replication-Defective Recombinant VSV

VSV that lacks the G protein function and which express 1L-12 or IFN-β and γ are constructed. Such viruses are generated in helper cells (CHO) that have been constructed to inducibly express the VSV G protein. Following infection of target cells, resultant viruses infect cells because they contain the VSV G from the helper cell. However, following infection and replication, progeny viruses will lack the receptor G and cannot disseminate to infect surrounding tissue. It is likely that these viruses are able to infect tumor cells and preferentially replicate to express immunomodulatory or suicide protein. Replication-defective VSV viruses expressing selected heterologous genes are produced and compared regarding their anti-tumor efficacy in vitro and in vivo against wt VSV counterparts. rVSV lacking M and N protein function are produced. Additionally, VSV having a replacement of the G protein with other receptors, such as tumor specific receptors are produced and analyzed to determine if the presence of the tumor specific receptor in the rVSV is more tumor cell specific.

### Example 12

### Vesicular Stomatitis Virus Expressing Tumor Suppressor p53

### Materials and Methods

Cells and Transfection.

C57BL/6 and 129/B6 mouse embryonic fibroblasts (MEFs) were maintained in Dulbecco's modified Eagle's medium (DMEM) supplemented with 10% fetal bovine serum (FBS), 5% penicillin-streptomycin, and 1% nonessential amino acids. B16(F10) cells were maintained in DMEM supplemented with 10% FBS, 5% penicillin-streptomycin, and 1.5 g/liter sodium bicarbonate. BHK cells were maintained in DMEM supplemented with 10% FBS and 5% penicillin-streptomycin; TS/A and TS/A-luc cells were maintained in RPMI 1640 supplemented with 10% FBS, 5% penicillin-streptomycin, and 10 mg/ml puromycin for TS/A-luc. C57BL/6 MEFs were transfected using Lipofectamine LTX and Plus reagents (Invitrogen); all other cell lines were transfected using Lipofectamine 2000 (Invitrogen) according to the manufacturer's protocol.

Generation of VSV-mp53 and VSV-DM-mp53:

In order to obtain the mp53 cDNA, PCR was performed using Pfx super mix (Invitrogen) and the oligonucleotides FWD (5′-TTATGTCGACATGACTGCCATGGAGGAGTC-3′) and REV (5′-GCTAGCAGCCCTGAAGTCATA-3′) with the pORF-mp53 construct as a template (Invivogen). The PCR product was then ligated into pCR-Blunt II Topo (Invitrogen), and the sequence was verified. mp53 was then digested using SalI and NheI restriction enzymes (NEB) and gel purified. mp53 was then cloned into the XhoI and NheI sits of VSV-XN2 and VSV-M(mut). VSV-M(mut) was generated by mutating amino acids 52 to 54 of the M protein from DTY to AAA using a site-directed mutagenesis kit (Stratagene) with the following oligonucleotides: for amino acid 52 D to A, FWD, 5′ GGAGTTGACGAGATGGCCACCTATGATCCGAATC, and REV, 5′ GATTCGGATCATAGGTGGCCATCTCGTCAACTCC; for amino acid 53 T to A, FWD, 5′ GGAGTTGACGAGATGGACGCCACCTATGATCCGAATC, and REV, 5′ GATTCGGATCATAGGTGGCGTCCATCTCGTCAACTCC; and for amino acid 54 Y to A, FWD, 5′ TTTGGAGTTGACGAGATGGACACCGCTGATCCGAATCAATTAAG, and REV, 5′ CTTAATTGATTCGGATCAGCGGTGTCCATCTCGTCAACTCCAAA. Virus was then grown using a virus recovery protocol described previously (Lawson N. D., et al. Proc. Natl. Acad. Sci. U.S.A. 92:4477-4481). Purification and concentration were achieved using sucrose centrifugation. Viral titers were obtained using the standard plaque assay.

Virus Infections:

MEFs and TS/A and B16(F10) cells were seeded in 6 or 12-well plates and grown to 70% confluence. After washing with 1× phosphate-buffered saline (PBS), the cells were infected with rVSVs at the indicated multiplicity of infection (MOI), which was calculated as follows: (number of cells×MOI)/titer=ml virus needed. The cells were incubated with virus for 1 h at 37° C. in serum-free DMEM with rocking every 15 min. The cells were washed with PBS twice, and complete medium was added to the cells.

Cell Viability:

C57BL/6 MEFs and TS/A and B16(F10) cells were grown to 70% confluence in 12-well plates. The cells were then infected as described above at the indicated MOIs. After incubation for 6, 12, 24, and 48 h, cells were collected, washed with PBS twice, and suspended in annexin V buffer (BD; 51-66121E). The cells were then stained using annexin V-eFluor 450 (1 μg/ml; eBioscence) and propidium iodide (1 μg) and then used for flow cytometry. Cells were considered dead when they shifted from being annexin V single positive (early apoptotic) to annexin V-propidium iodide double positive (late apoptotic).

Western Blotting:

Infected cells were collected and lysed in radioimmunoprecipitation assay (RIPA) buffer containing protease and phosphatase inhibitors. Protein concentrations were determined using Coomassie Plus Protein Assay Reagent (Thermo; 1856210), and the optical density (OD) was read at 595 nm. Equal amounts of protein were subjected to SDS-PAGE, transferred onto a polyvinylidene difluoride (PVDF) membrane, and blocked using 5% milk in PBS-Tween (0.1% Tween 20). The membranes were then immunoblotted using antibodies against p53 (Santa Cruz; SC-99), p53 phosphorylated at serines 18 and 389 (Cell Signaling; 9284 and 9281), and VSV-G (Sigma; V5507). The membranes were probed with a secondary antibody, goat anti-mouse, goat anti-rabbit, or donkey anti-goat (Santa Cruz), at 1:5,000. The image was resolved using chemiluminescence (Super Signal) and captured by autoradiography (Kodak Film).

p53 Luciferase Assay:

Firefly p53-luc (Clontech; 250 ng) and Renilla pRLTK (50 ng) were transfected as described previously into 70% confluent C57BL/6 MEFs and TS/A and B16(F10) cells grown in 12-well plates 4 h prior to infection. The cells were then infected as described above at the desired MOI. After 24 h, the cells were lysed using 1× luciferase cell culture lysis reagent (CCLR), and luciferase assays were performed using a Luciferase Assay System and a Renilla Assay System (Promega).

p53 DNA n′ Zicroarray:

C57BL/6 MEFs and TS/A cells were grown to 70% confluence in 6-well plates and infected as described previously at the indicated MOI. After 8 h incubation, total RNA was collected using an ArrayGrade Total RNA Isolation Kit (SA Biosciences). cDNA and biotin-labeled cRNA were then generated using TrueLabeling-AMP 2.0 (SA Biosciences). The labeled cRNA was then hybridized to an Oligo GEArray DNA Microarray (OMM-027) and resolved using chemiluminescent detection and autoradiograph capture.

Mouse Studies:

Female BALB/c mice (6 to 8 weeks old) were acquired from Jackson Laboratory. Female BALB/c athymic nude (CAnN.Cg-Fox1^{nu}/Crl) mice were acquired from Charles River. All mice were housed under pathogen-free conditions.

Toxicity:

Female BALB/c (n=7) or nude (n=5) mice were injected with VSV intravenously (i.v.) in 100 μl PBS in the tail vein or in 10 μl PBS intranasally (i.n.). The mice were then monitored for survival. Additionally, mice were sacrificed if they displayed gross morbidity or if they developed hind limb paralysis.

Tumor Studies:

BALB/c (n=7) or nude (n=5) mice were injected with 1×10⁵ TS/A-luc cells i.v. Three days later, the mice were injected with 5×10⁷ or 5×10⁸ PFU of rVSV. The survival of the mice was monitored daily. The surviving mice were rechallenged with 2×10⁵ TS/A-luc cells and monitored daily for survival.

ELISPOT:

The protocol for the enzyme-linked immunospot (ELISPOT) assay was followed. Female BALB/c mice (n=5) were injected i.v. in the tail vein with 1×10⁵ TS/A-luc cells. Three days later, the mice were infected with 5×10⁷ PFU of the indicated rVSV i.v., and 10 days postinfection, the mice were sacrificed and the spleens were removed. TS/A-luc cells treated with mitomycin C (25 μg/ml) were used as the target. The plates were analyzed the following day using an ELISPOT reader.

Multiplex ELISA and Flow Cytometry:

Female BALB/c (n=6) mice were infected with 5×10⁷ PFU rVSV i.v. Twenty-four (n=3) and 96 (n=3) hours postinfection, the sera, spleens, and thymuses were collected. The sera were stored at −80° C. and used for multiplex enzyme-linked immunosorbent assay (ELISA) (Millipore). The sera were incubated overnight at 4° C. with agitation with a mix of antibody-coated beads. The next day, samples were incubated with detection antibodies for 1 h at room temperature and run on a Luminex 100 machine. The spleens and thymuses were passed through a 0.2-μm mesh, and cells were recovered in 3 ml of RPMI 1640. The cells were then centrifuged at 1,400 rpm, and red blood cells were lysed using 1 ml of ACK buffer (red blood cell lysis buffer [0.15 M NH₄Cl, 1 M KHCO₃, 0.1 mM Na₂ EDTA]) for 5 min, centrifuged again, and washed with 1×PBS. The cells were counted, and two groups of 1×10⁶ cells were stained for 4-color flow cytometry using CD4-phycoerythrin (PE) (BD 557308), CD8-peridinin chlorophyll protein (PerCP) (BD 553036), B220-fluorescein isothiocyanate (FITC) (BD 553088), CD49b-allophycocyanin (APC) (eBioscience 17-5971-81), CD11c-FITC (BD 553801), CD11b-APC (BD553312), F4/80-PE (eBioscience 12-4801-80), or major histocompatibility complex class II (MHC-II)-eflour450 (eBioscience 48-5321-80) on ice in darkness for 20 min. The cells were then washed once with PBS and fixed with 1 to 4% paraformaldehyde and stored in the dark at 4° C. until samples were run on flow cytometry (BD-LSRI).

IFN-β ELISA:

BALB/c mice (n=3) were mock infected or infected with 5×10⁷ PFU rVSV. Six hours postinfection, blood was collected by submandibular bleeding. The blood was then incubated for 1 h at 4° C. and centrifuged at 13,000 rpm in order collect serum. Samples were diluted 1:50 and assessed by IFN-β ELISA (PBL Interferonsource).

In Vivo Imaging of Mice:

Mice were imaged as previously described (Balachandran S., Barber G. N. 2004. Defective translational control facilitates vesicular stomatitis virus oncolysis. Cancer Cell 5:51-65).

Statistics:

All statistical analysis was performed using GraphPad Prism 5.

Results

Generation of Oncolytic VSV-M(Mut)-Mp 53 and VSV-Mp 53.

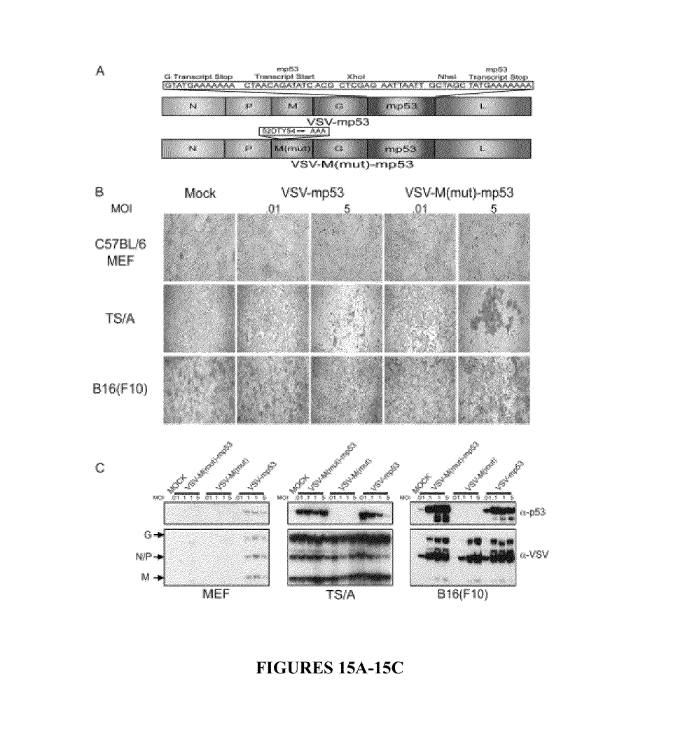

To determine whether novel VSVs expressing p53 have an improved ability to modulate innate and adaptive immune responses that may result in increased antitumor efficacy and safety in vivo, the mp53 gene was cloned between the G and L proteins of VSV (VSV-XN2). As an additional experiment, murine p53 was similarly cloned into VSV harboring a mutation in the M protein [VSV-M(mut)] so that the resultant virus could not block host mRNA export, including innate immune-related transcripts that could conceivably be triggered by heterologous p53 activity. The resulting plasmids were then used to recover functional viral particles. (FIG. 15A). To verify that the resulting VSV-M(mut)-m and VSV-mp53 retained oncolytic specificity, normal C57BL/6 MEFs or tumor [murine mammary adenocarcinoma TS/A or murine melanoma B16(F10)] cells were infected at an MOI of 0.01, 0.1, 1, and 5 for 24 h. Cytopathic effect (CPE) was observed using bright-field microscopy (FIG. 15B). These results indicated that only the TS/A and B16(F10) tumor lines exhibited the characteristics of infection as manifested by CPE, in contrast to the normal MEFs. The MEFs and tumor cells were then lysed and examined by immunoblotting to confirm the expression of the mp53 transgene (FIG. 15C). This analysis indicated that both VSV-mp53 and VSV-M(mut)-mp53 efficiently expressed the mp53 transgene in tumor cell lines [TS/A and B16(F10)] permissive for VSV replication. These data emphasize that VSV can efficiently express the p53 gene and that normal MEFs do not support robust viral replication.

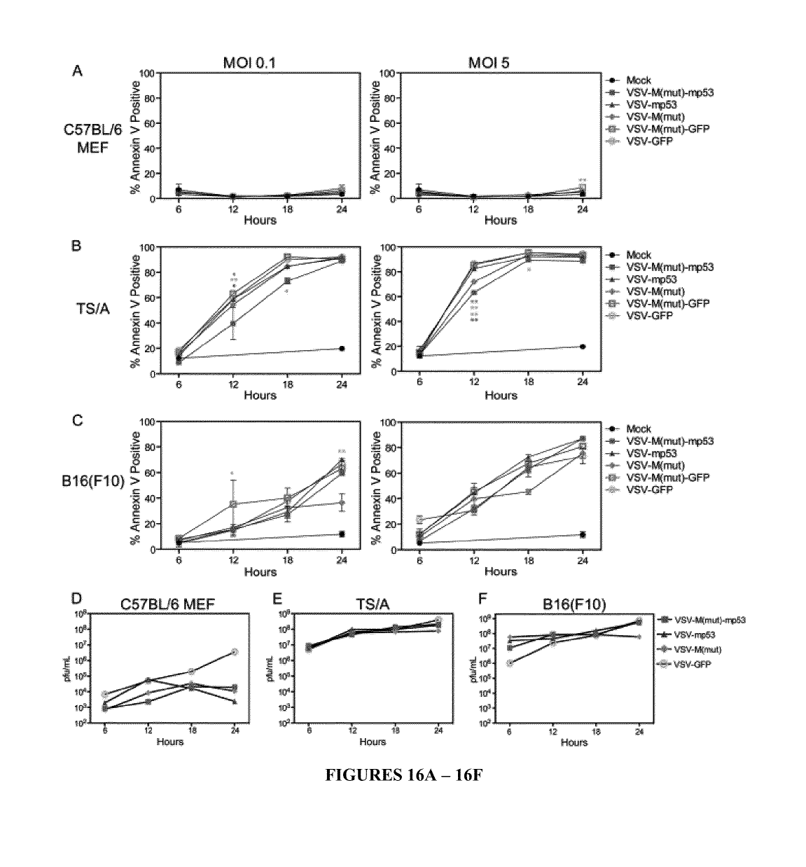

To further evaluate whether VSV-mp53 or VSV-M(mut)-mp53 retained the ability to replicate efficiently in and kill transformed cells, normal MEFs and TS/A or B16(F10) cells were infected with the above-mentioned rVSVs. The results indicated that VSV-mp53 and VSV-M(mut)-mp53, as well as control VSV-green fluorescent protein (GFP) and VSV-M(mut)-GFP, effectively killed TS/A and B16(F10) cells and replicated to similar titers within 24 h (FIGS. 16A-16C). Importantly, VSV-mp53 and VSV-M(mut)-mp53 retained oncolytic specificity, since MEFs remained largely unaffected by rVSV infection (FIG. 16A). Indeed, MEFs generated 2 to 3 log units less virus as measured 24 h postinfection than TS/A or B16(F10) cells (FIGS. 16D to 16F). Moreover, viruses expressing p53 replicated even less in MEFs than control VSV-GFP. This analysis indicates that MEFs are not robustly permissive to VSV infection compared to transformed TS/A and B16 cells. This may be due to MEFs having intact innate immune pathways that could combat infection. These data clearly evidence that insertion of mp53 into either the XN2 or M(mut) background does not inhibit the replicative or oncolytic abilities of these viruses in vitro.

Expressed Mp 53 is Phosphorylated and Activates Transcription of Target Genes.

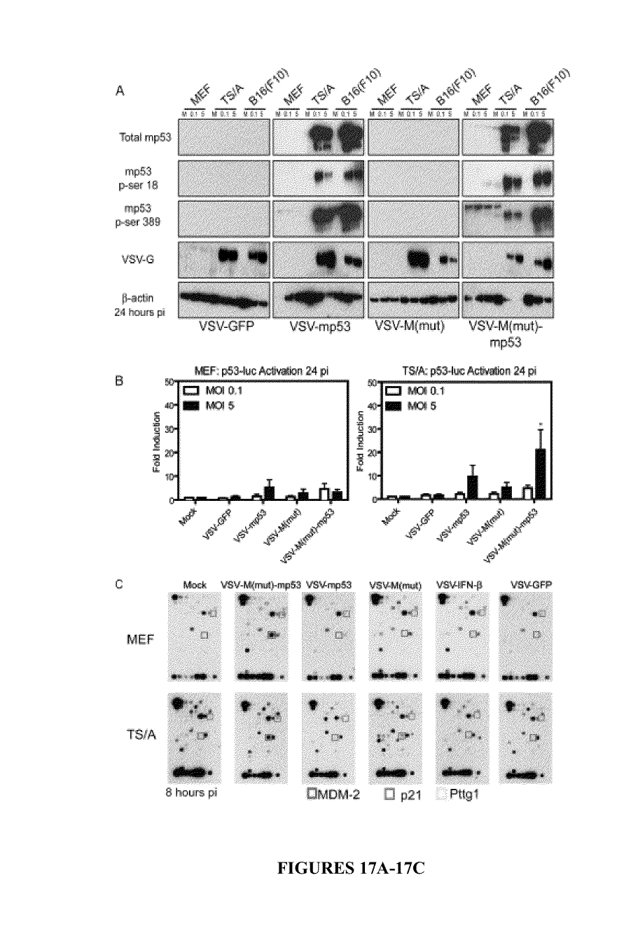

It is known that mp53 undergoes a variety of posttranslational modifications, such as phosphorylation, ubiquitination, sumoylation, acetylation, and neddylation, in order to regulate its activity. For example, murine p53 can be activated by phosphorylation on serine 18. This event is carried out by DNA-dependent protein kinase (DNA-PK) in response to cellular stress, which causes dissociation of p53 from its negative regulator, MDM-2 (an E3 ubiquitin ligase), and allows p53 stabilization. In addition, serine 389 can be phosphorylated, which induces the p53 oligomerization necessary for DNA binding and transcription activation. To evaluate whether mp53 expressed by VSV-mp53 or VSV-M(mut)-mp53 was active, mock- or rVSV-infected cell lysates were immunoblotted using phosphorylation-specific murine p53 antibodies. This analysis confirmed efficient expression of the mp53 transgene that correlated well with the ability of rVSV to preferentially replicate in transformed cells (FIG. 17A). Additionally, membranes were probed with phosphoserine 18- or 389-specific antibodies to determine if virus-expressed mp53 is phosphorylated on serine residues necessary for stabilization and transcriptional activation (FIG. 17A). Confirmation that mp53 is indeed phosphorylated on serines 18 and 389 provided initial evidence that mp53 produced during viral replication is capable of being activated and may be able to initiate the transcription of target genes. Next, to ascertain if VSV-expressed phosphorylated mp53 is transcriptionally active, a p53 luciferase reporter assay was performed (FIG. 17B). Essentially, VSV-mp53 and VSV-M(mut)-mp53 were used to infect normal or tumor cells transiently expressing a luciferase reporter gene under the control of a p53 promoter element. This analysis indicated that permissive TS/A cells exhibited an increase in luciferase expression compared to normal MEFs following infection with VSVs expressing p53. This is likely due to TS/A cells supporting high levels of viral replication. However, MEFs also exhibited some luciferase expression after infection with rVSVs expressing p53, suggesting that modest amounts of heterologous p53 may be being produced in these cells, albeit at very low levels. However, it has been shown that VSV infection can also modestly induce the activation of mp53 through a cross talk mechanism involving the type I IFN response. An important observation from this experiment was that luciferase expression was higher in cells infected with VSV-M(mut)-mp53, presumably because more luciferase mRNA was able to escape from the nucleus for translation (FIG. 17B). To extend this examination further, the ability of virus-encoded mp53 to activate transcription was additionally assessed using a p53 microarray harboring 112 p53-inducible genes (FIG. 17C). MEFs and TS/A cells were infected at an MOI of 10 or 1, respectively, for 1 h and lysed 8 h post-infection to retrieve cellular mRNA. The result indicated that infection with VSV-M(mut)-mp53 induced the robust production of the known p53-inducible MDM-2 (red boxes) and p21 (blue boxes) genes (FIG. 17C). Presumably, most of the other genes on the panel were not detected, since p53 may only weakly regulate these genes. One gene, encoding PTTG1 (pituitary tumor-transforming 1; yellow boxes) was observed to be induced in normal MEFs infected with VSV-M(mut) or VSV-IFN-β, possibly because it has interferon-inducible transcription sites in its promoter region. These data highlight the potential advantage of using the VSVs with a defective matrix (M) protein, since MDM-2 and p21 mRNA induction were only robustly observed in cells infected with VSV-M(mut)-mp53, which cannot block host cell mRNA export. Additionally, these data confirm that virus-expressed murine p53 especially from VSV-M(mut), not only is appropriately posttranslationally modified, at least by phosphorylation on serines 18 and 389, but is transcriptionally active.

VSV-M(mut)-Mp 53 is Highly Attenuated in BALB/c Mice.

To start to evaluate the in vivo effects of VSV-ΔM-mp53, we carried out preliminary toxicity assays using murine models. BALB/c mice (n=7) were infected i.v. with increasing doses of 1×10⁸ to 1×10⁹ PFU of VSV-M(mut)-mp53, VSV-mp53, VSV-M(mut)-GFP, VSV-GFP, or VSV-IFN-β (Table 6). Mice infected with VSV-GFP exhibited toxicity at doses of 1×10⁸ to 5×10⁸. In contrast, VSV-mp53, VSV-M(mut), and VSV-M(mut)-GFP appeared more attenuated in mice (5×10⁸ or 1×10⁹ PFU). Surprisingly, 84% (6/7) of the mice treated with VSV-M(mut)-mp53 compared to 14% (1/7) of the mice treated with VSV-M(mut) survived infection with 1×10⁹ PFU [one died shortly after inoculation with VSV-M(mut)-mp53, but not through virus-induced encephalitis]. This experiment highlighted that mp53 expression in an M-defective VSV perhaps had an additive effect, since mice infected with VSV-ΔM or VSV-mp53 exhibited increased mortality compared to VSV-M(mut)-mp53. These data demonstrated that VSV-M(mut)-mp53 is highly attenuated and yet retains in vitro oncolytic activity and could thus be suitable for examination as a cancer therapeutic using in vivo tumor models.

TABLE 6
VSV-M(mut)-mp53 is highly attenuated in vivo

| | rVSV toxicity in BALB/c mice^{a} | | | |
|---|---|---|---|---|
| | Dose | | | |
| Virus | (PFU/mouse) | Route | Mortality | % Mortality |
| VSV-M(mut)-mp53 | 1 × 10⁸ | I.v. | 0/7 | 0 |
| VSV-mp53 | 1 × 10⁸ | I.v. | 0/7 | 0 |
| VSV-M(mut) | 1 × 10⁸ | I.v. | 0/7 | 0 |
| VSV-GFP | 1 × 10⁸ | I.v. | 1/7 | 14 |
| VSV-M(mut)-mp53 | 5 × 10⁸ | I.v. | 0/7 | 0 |
| VSV-mp53 | 5 × 10⁸ | I.v. | 5/7 | 71 |
| VSV-M(mut) | 5 × 10⁸ | I.v. | 5/7 | 71 |
| VSV-M(mut)-GFP | 5 × 10⁸ | I.v. | 5/7 | 71 |
| VSV-GFP | 5 × 10⁸ | I.v. | 7/7 | 100 |
| VSV-M(mut)-mp53 | 1 × 10⁹ | I.v. | 1/7 | 14 |
| VSV-mp53 | 1 × 10⁹ | I.v. | 7/7 | 100 |
| VSV-M(mut) | 1 × 10⁹ | I.v. | 6/7 | 86 |
| VSV-M(mut)-GFP | 1 × 10⁹ | I.v. | 7/7 | 100 |
| VSV-GFP | 1 × 10⁹ | I.v. | 7/7 | 100 |

^{a}BALB/c mice (n = 7) were infected i.v. with increasing doses of rVSV, and mortality was monitored. Log rank tests results were as follows: for mice treated with 1 × 10⁸ PFU, P = 0.0042; for mice treated with 5 × 10⁸ PFU, P = 0.0256; for mice treated with 1 × 10⁹ PFU, P = 0.0257.

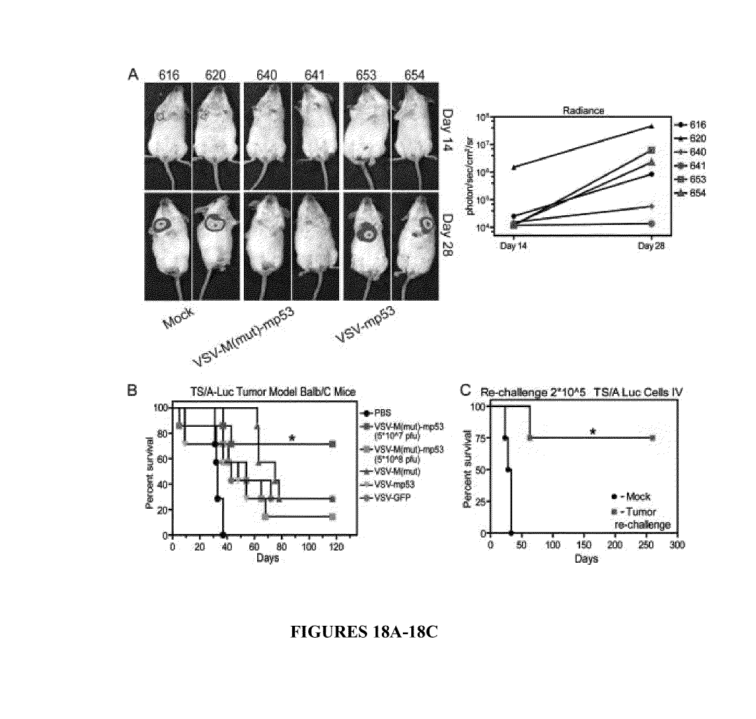

To assess the antitumor efficacy of VSV-M(mut)-mp53 and VSV-mp53, we chose a BALB/c syngeneic tumor model using TS/A mammary adenocarcinoma cells stably transfected with a luciferase reporter (TS/A-luc) developed by our laboratory. Wild-type BALB/c mice (n=7) were injected i.v. with 1×10⁵ TS/A-luc cells. Three days later, the mice were treated with 5×10⁷ or 5×10⁸ PFU of VSV-M(mut)-mp53, mock treated, or treated with 5×10⁷ PFU of control virus VSV-GFP, VSV-mp53, or VSV-M(mut). One advantage of TS/A-luc is that this reagent allows in vivo monitoring of tumor growth using an in vivo imaging system (IVIS). Measurement of luciferase activity can thus be used to assess the relative tumor burden. Representative images, along with luciferase signal quantification, from days 14 and 28 after tumor administration are shown for mock-, VSV-M(mut)-mp53-, and VSV-mp53-treated mice (FIG. 18A). Noticeably, luciferase activity is absent in mice treated with VSV-M(mut)-mp53 up to 28 days after tumor inoculation. This strategy, using a single dose (5×10⁷ PFU) of VSV-M(mut)-mp53, significantly protected BALB/c mice from TS/A-luc tumor-induced death compared to similar doses of control rVSVs (n=7; P=0.0045). Mice treated with VSV-M(mut), VSV-mp53, VSV-GFP, and an increased dose (5×10⁸ PFU) of VSV-M(mut)-mp53 had median survival times ranging from 40 to 75 days. However, less than 50% of VSV-M(mut)-mp53 mice treated with 5×10⁷ PFU succumbed over 120 days posttreatment, and thus, a mean could not be determined (FIG. 18B).

Because VSV-M(mut)-mp53 exhibited low toxicity, we also hypothesized that using an increased dose of agent would lead to enhanced antitumor clearance. However, treatment with 5×10⁸ PFU VSV-M(mut)-mp53 protected only 14% of animals compared to treatment at a lower dose (5×10⁷ PFU), at which approximately 71% of mice survived (FIG. 18B). It is thus possible that the increased viral presence biases the immune response to the excess of viral antigens rather than tumor cell antigens. The stimulation of antitumor cell activity by VSV oncolytic therapy is known to greatly facilitate tumor cell clearance. Surviving mice previously treated with VSV-M(mut)-mp53 (n=4) were rechallenged with 2×10⁵ TS/A-luc cells to determine if lasting immunity against the tumor was apparent. These results indicated that immunologic memory is indeed generated against TS/A-luc by VSV-M(mut)-mp53 and that tumor clearance is likely facilitated by the host immune response, since 75% of the mice survived tumor rechallenge up to an additional 250 days until sacrifice. One of the rechallenged mice did succumb to tumor regrowth, however, likely indicating immune escape of the tumor, but only after doubling the median survival time of the naïve mice (60 versus 30.5 days; P=0.004) (FIG. 18C).

VSV-M(Mut)-Mp 53 Stimulates Antitumor Immunity.

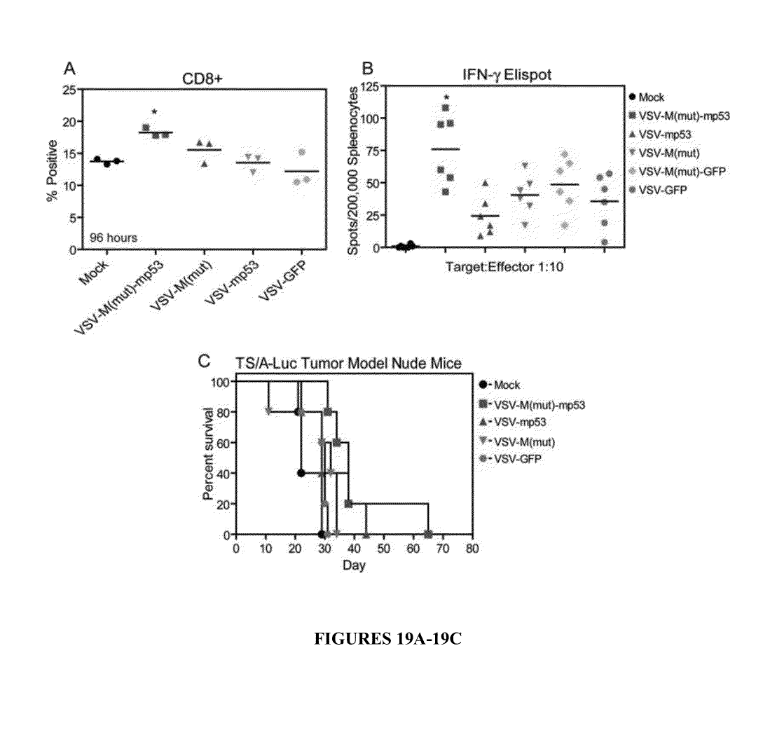

This analysis indicated that antitumor immune responses were generated by mice with TS/A treated with VSV-M(mut)-mp53 (FIG. 18C). It was therefore sought to determine if VSV-M(mut)-mp53 was able to modulate the immune compartment and host antitumor responses (FIGS. 19A and 19B). Mice were inoculated (n=6) with VSV-M(mut)-mp53, VSV-M(mut), VSV-mp53, or VSV-GFP and measured splenic CD8⁺ cells (FIG. 19A). An ELISPOT assay was then performed using mitomycin C-treated TS/A-luc cells as targets to determine if VSV-M(mut)-mp53 increased the number of tumor-specific IFN-γ-secreting T cells compared to control viruses (FIG. 19B). Indeed, tumor-bearing mice that received VSV-M(mut)-mp53 had elevated numbers of tumor-specific CD8⁺ T cells compared to controls receiving VSV-M(mut) and VSV-GFP. We then repeated the toxicity and tumor model experiments in athymic BALB/c nude mice to further establish the necessity for an intact adaptive immune compartment for complete tumor clearance. It has previously been reported that mice lacking T cells succumb to i.v. and i.n. VSV infection at low doses approximately 3 to 4 weeks after exposure. To determine the role of T cell activity in p53 action, athymic BALB/c nude mice (n=5) were infected i.v. with 5×10⁷ PFU or i.n. with 5×10⁸ PFU VSV-ΔM-mp53, VSV-mp53, or control rVSVs, and their survival was monitored for 80 or 120 days (Table 7). Surprisingly, these mice tolerated i.v. treatment with 5×10⁷ PFU of VSVM(mut) or VSV-M(mut)-mp53. This analysis indicates that protection from modest i.v. doses of VSV-M(mut)-mp53 is likely due to modulation of the innate immune response, possibly involving the production of type I IFN, and not T cells. It is known that p53 can be induced by IFN signaling, can enhance IFN signaling, and can play a role in antiviral responses to VSV, since p53 knockout mice display increased susceptibility to VSV infection. Interestingly, however, all Nude mice succumbed to rVSVs following i.n. inoculation (Table 7). This may be due to rapid VSV transmission to the central nervous system (CNS) via olfactory nerve infection, resulting in increased morbidity. Next assessed was the rVSV antitumor efficacy using TS/A-luc in athymic BALB/c nude (n=5) mice with the treatment schedule previously described (FIG. 19C). All nude mice treated with rVSV succumbed to tumor formation, highlighting the requirement for an intact T cell compartment in eliciting protection from TS/A-luc tumor formation.

TABLE 7
VSV-M(mut)-mp53 toxicity in nude mice

| | rVSV Toxicity in BALB/c nude mice^{a} | | | |
|---|---|---|---|---|
| | Dose | | | |
| Virus | (PFU/mouse) | Route | Mortality | % Mortality |
| VSV-M(mut)-mp53 | 5 × 10⁷ | I.v. | 0/5 | 0 |
| VSV-mp53 | 5 × 10⁷ | I.v. | 5/5 | 100 |
| VSV-M(mut) | 5 × 10⁷ | I.v. | 0/5 | 0 |
| V8V-GFP | 5 × 10⁷ | I.v. | 3/5 | 60 |
| VSV-M(mut)-mp53 | 1 × 10⁹ | I.v. | 3/5 | 60 |
| VSV-M(mut)-mp53 | 5 × 10⁸ | I.n. | 5/5 | 100 |
| VSV-mp53 | 5 × 10⁸ | I.n. | 5/5 | 100 |
| V8V-M(mut) | 5 × 10⁸ | I.n. | 5/5 | 100 |
| VSV-GFP | 5 × 10⁸ | I.n. | 5/5 | 100 |

^{a}Athymic BALB/c nude mice (n = 5) were infected with 5 × 10⁷ PFU i.v. and 5 × 10⁸ PFU i.n. and observed for mortality.

In addition to modulating CD8⁺ T cells, it was also possible that treatment with VSV-M(mut)-mp53 could alter additional antitumor immune effector mechanisms, including cytokine profiles, in treated mice. To further elucidate the mechanisms of protection and reduced toxicity manifested by VSV-M(mut)-mp53, it was sought to characterize the spleens and cytokine profiles of mice after rVSV administration. In addition to CD8⁺ T cells (FIG. 19A), the population of CD49b⁺cells, an NK cell marker, also increased in the spleens of mice infected with VSV-M(mut)-mp53 96 h postinfection. Serum cytokine levels were then tested using multiplex and IFN-β ELISAs. Treatment with VSV-ΔM-mp53 or control rVSVs led to the expected increases in antiviral-induced cytokines, such as tumor necrosis factor alpha (TNF-α) and RANTES. However, treatment with VSV-M(mut)-mp53 appeared to blunt the production of inflammatory cytokines, such as interleukin 6 (IL-6) and IP-10, since reduced levels were observed in VSV-M(mut)-mp53-infected mouse serum. IL-6 has been shown to facilitate angiogenesis and tumor cell growth, indicating that its suppression may benefit tumor clearance. IP-10 is a proinflammatory chemoattractant that induces immune cell infiltration in response to viral infection and may have antitumor effects in some situations. Moderated levels of IP-10 may allow a more optimal antitumor response to occur and mitigate some infiltration of immune cells, possibly reducing CNS toxicity. Furthermore, VSV-ΔM-mp53 significantly increased the amount of IFN-α present in the serum 6 h postinfection. The cytokine profiles in these mice may begin to explain the reduced toxicity displayed by VSV-M(mut)-mp53, for example, by limiting inflammatory responses and increasing IFN-β production, which could limit virus spread as well as facilitate increased tumor clearance.

Discussion

Here, the data clearly demonstrate that the insertion of mp53 into the VSV-M(mut) background produces a highly attenuated and effective rVSV. It is well known that rVSV preferentially replicates in and kills transformed cells in vitro and in vivo, and VSV-M(mut)-mp53 retains this ability. Furthermore, the mp53 transgene appears functional and is at least phosphorylated on serines 18 and 389. As described above, phosphorylation on serine 18 enables dissociation of mp53 from MDM-2, an E3 ubiquitin ligase that negatively regulates mp53 by targeting it for proteasomal degradation. Phosphorylation on serine 389 allows oligomerization and DNA binding to occur, which are necessary steps for transcription activation of p53. Phosphorylated mp53 expressed from rVSV activated a luciferase reporter gene under the control of a p53-responsive promoter more potently when expressed from the VSV-M(mut) background. This included clearly enhanced expression of MDM-2 and p21 mRNAs, which are known mp53 targets. The tumor suppressor p53 has the ability to exert many effects when transcriptionally active in cells. For example, p53 reactivation in hepatocellular carcinoma led to the induction of innate immune signaling and to tumor regression. It has also been reported that p53 can cross talk with the IFN system and plays an essential role in antiviral immunity. IFN-inducible ISGF3 can activate the transcription of p53, and TLR3 production can be initiated by p53. Additionally, IFN treatment can activate apoptotic pathways in both p53-dependent and -independent manners. Interestingly, mice that have an extra copy of the p53 gene (“super p53” mice) are remarkably resistant to VSV infection, whereas p53 knockout mice are highly susceptible to infection, again highlighting the potential role of p53 in antiviral immunity. Furthermore, p53 is known to regulate the cell cycle, apoptosis, microRNAs (miRNAs), and senescence, all of which could have a benefit when expressed from VSV-M(mut)-mp53 in the tumor microenvironment.

VSV-M(mut)-mp53 was highly attenuated in vivo and showed potent ability to clear metastatic disease from immunocompetent BALB/c mice. The reduced toxicity associated with the virus allowed administration of 20 times more virus without significant mortality. However, increasing the treatment dose 10-fold to 5×10⁸ PFU did not increase VSV-M(mut)-mp53 antitumor efficacy. It is possible that by increasing the viral load the secondary immune response becomes more heavily focused on viral antigens and cannot respond efficiently to tumor cell antigens due to being overwhelmed. Furthermore, it is possible that infection at higher doses may lead to an increase in regulatory T cells or myeloid-derived suppressor cells (MDSC), which could dampen the antitumor response. Surprisingly, VSV-M(mut)-mp53 appears attenuated in athymic BALB/c nude mice following i.v. inoculation, indicating that viral clearance is not completely dependent on T cells and that VSV-M(mut)-mp53 likely modulates the innate immune response in order to control viral infection.

It has previously been shown that oncolytic virotherapy using VSV, as well as other viruses, can lead to cross-presentation of tumor antigens and induction of a host antitumor response. We therefore determined whether VSV-M(mut)-mp53 treatment might modulate components of the innate and adaptive immune systems in order to elicit a cross-presentation response and facilitate tumor clearance. Indeed, mice treated with VSV-M(mut)-mp53 showed increased numbers of tumor-specific CD8⁺ T cells and CD49b⁺ NK cells in the spleen 96 h postinfection. Increasing the number of CD49b⁺ NK cells is advantageous, as these cells have cytolytic abilities and can directly kill virus-infected tumor cells. This also leads to the production of tumor cell debris, which can be processed and displayed by antigen-presenting cells, leading to the generation of tumor-specific CD8⁺ T cells that can enhance tumor protection. It is thus not surprising that athymic BALB/c nude mice were not protected from TS/A-luc tumor formation, since these mice lack T cells.

As mentioned above, VSV-M(mut)-mp53 was found to modulate the cell population in the spleen, as exhibited by CD49b⁺ cells. VSV-M(mut)-mp53 also affected the serum levels of cytokines and chemokines in infected animals. Normally, viral infection leads to production of inflammatory and antiviral cytokines and chemokines, like IL-6, IP-10, TNF-α, and IFN-β. However, treatment with high doses of rVSV can lead to increased expression of these molecules, which can cause lethality by inducing a fatal cytokine storm. The data herein, evidence that VSV-M(mut)-mp53 administration led to a reduction in the serum levels of IL-6 and IP-10 24 h after treatment while serum IFN-β levels were elevated 6 h postinfection. mp53 has been reported to be a potential negative regulator of IL-6 and suppressor of macrophage. IL-6 can act as a proinflammatory cytokine that signals through the IL-6rα-gp130 receptor complex and activates JAKs, which in turn activate a number of STAT molecules. IL-6 is also the primary mediator of the acute-phase fever response and could be a component of inflammatory responses seen with increased doses of rVSV. IP-10 (CXCL10) levels were also reduced in the sera of VSV-M(mut)-mp53-treated mice. IP-10 is a proinflammatory chemokine that functions as a chemoattractant for macrophages, dendritic cells (DC), and NK cells by binding to CXCR3. The reduction in serum IP-10 may reduce immune cell infiltration into sites where the furtherance of inflammation may be deleterious to the host. Furthermore, the decreased inflammation associated with VSV-M(mut)-mp53 could allow a more finely tuned antitumor response to occur by not biasing the immune response against the viral infection. Thus, it is possible that a reduction in the expression of IL-6 and IP-10, along with the suppression of macrophage activation in the presence of increased levels of IFN-β, may facilitate a reduction in VSV-M(mut)-mp53 toxicity and enhance antitumor efficacy. However, it is also important to note that the innate immune system can have disparate effects on oncolytic viruses. For example, rVSV has been combined with pretreatment histone deacetylase inhibitors, which can effectively blunt the antiviral response and facilitate increased tumor oncolysis. It has also been observed that the innate immune response can have negative impacts on the effects of other oncolytic viruses. Therefore, our results may be limited to the VSV-M(mut)-mp53 construct and the model systems used. Nevertheless, our data indicate that VSV-M(mut)-mp53 is a potent oncolytic vector with enhanced safety and efficacy. Thus, VSV-M(mut)-mp53 could be considered for further studies to examine the potential of this therapy as a future anticancer treatment.

### Example 13

### VSV Expressing Gp160 as an Oncolytic Target for CD4⁺ Tumors

VSV is used as an oncolytic agent for the treatment of malignant disease. VSV preferentially replicates in tumor cells and destroys them by apoptosis since anti-viral defense mechanisms are defective. However, in normal cells, anti-viral innate immune mechanisms are intact and VSV cannot replicate efficiently. The glycoprotein of VSV (G) is tropic for many cell types making VSV attractive for the treatment of many different types of tumors. The VSV vector described here specifically targets select tumor types.

The VSV G sequence was replaced by an HIV envelope nucleic sequence which encodes for and expresses the HIV gp160 protein. The gp160 specifically targets CD4 positive tumors. CD4⁺ tumors include, adult T cell leukemia (ATL) and cutaneous lymphoma. VSV-gp160 was generated and was found to be selective for cell expressing the CD4 marker. Non-CD4 cells could not be infected. Normal CD4⁺ cells were able to prevent normal VSV replication they have intact innate immune systems. However, ATL cells are extremely susceptible to VSV infection, replication and lysis. The results obtained from the SCID mouse model show that the VSV-gp160 can clear human ATL cells from the animal without harming the animal itself. The VSV vector constructed comprise a gp160 (VSV-gp160) and also a VSV comprising gp160 and interferon (VSV-gp160 IFN).

The present invention is not to be limited in scope by the specific embodiments described herein. Various modifications of the invention in addition to those described herein will become apparent to those skilled in the art from the foregoing description and accompanying figures. Such modifications are intended to fall within the scope of the appended claims.

What is claimed:

1. A composition comprising a recombinant vesicular stomatitis (VSV) vector encoding a human immunodeficiency virus (HIV) gp160 nucleic acid sequence, mutants, variants or complementary DNA (cDNA) sequences thereof.

2. The composition of claim 1, wherein the VSV further comprises one or more foreign nucleic acid sequences or complementary DNA (cDNA) sequences thereof, for expressing one or more proteins or peptides.

3. The composition of claim 2, wherein the one or more foreign nucleic acid sequences or complementary DNA (cDNA) sequences thereof, comprise: interferons, ligands, HIV or simian immunodeficiency virus (SIV) molecules, immune related molecules, cytokines, chemokines, cellular products, cell surface expression or secretion products, cell signaling molecules, or combinations thereof.

4. The composition of claim 3, wherein immune related molecules comprise: T cell receptors or ligands thereof, co-stimulatory molecules or ligands thereof, immunoglobulins, cell surface expression markers or receptors thereof.

5. The composition of claim 3, wherein the interferons, cytokines, chemokines and cellular products comprise: interferon α-, β- and/or γ-, interleukins, growth factors, tumor necrosis factors, integrins, or combinations thereof.

6. The composition of claim 1 wherein the VSV vector further comprises one or more deletions or mutation in one or more VSV nucleic acid sequences.

7. The composition of claim 1, wherein the VSV vector is oncolytic.

8. A method of preventing or treating human immunodeficiency virus (HIV) comprising: administering to an at risk patient or a patient suffering from HIV and related disorders, a therapeutically effective amount of a composition comprising: a recombinant vesicular stomatitis (VSV) vector encoding a human immunodeficiency virus (HIV) gp160 nucleic acid sequence, mutants, variants or complementary DNA (cDNA) sequences thereof.

9. The method of claim 8, wherein the VSV further comprises one or more foreign nucleic acid sequences or complementary DNA (cDNA) sequences thereof, for expressing one or more proteins or peptides, comprising: interferons, ligands, HIV or simian immunodeficiency virus (SIV) molecules, immune related molecules, cytokines, chemokines, cellular products, cell surface expression or secretion products, cell signaling molecules, or combinations thereof.

10. The method of claim 9, wherein immune related molecules comprise: T cell receptors or ligands thereof, co-stimulatory molecules or ligands thereof, immunoglobulins, cell surface expression markers or receptors thereof.

11. The method of claim 9, wherein the interferons, cytokines, chemokines and cellular products comprise: interferon α-, β- and/or γ-, interleukins, growth factors, tumor necrosis factors, integrins, or combinations thereof.

12. The method of claim 8, wherein the VSV vector further comprises one or more deletions or mutation in one or more VSV nucleic acid sequences.

13. The method of claim 8, wherein the VSV vector is oncolytic.

14. A method of treating cancer comprising administering to a patient in need thereof, a therapeutically effective amount of a composition comprising: a recombinant vesicular stomatitis (VSV) vector encoding a human immunodeficiency virus (HIV) gp160 nucleic acid sequence, mutants, variants or complementary DNA (cDNA) sequences thereof.

15. The method of claim 14, wherein the VSV further comprises one or more foreign nucleic acid sequences or complementary DNA (cDNA) sequences thereof, for expressing one or more proteins or peptides, comprising: interferons, ligands, HIV or simian immunodeficiency virus (SIV) molecules, immune related molecules, cytokines, chemokines, cellular products, cell surface expression or secretion products, cell signaling molecules, or combinations thereof.

16. The method of claim 15, wherein immune related molecules comprise: T cell receptors or ligands thereof, co-stimulatory molecules or ligands thereof, immunoglobulins, cell surface expression markers or receptors thereof.

17. The method of claim 15, wherein the interferons, cytokines, chemokines and cellular products comprise: interferon α-, β- and/or γ-, interleukins, growth factors, tumor necrosis factors, integrins, or combinations thereof.

18. The method of claim 14, wherein the VSV vector further comprises one or more deletions or mutation in one or more VSV nucleic acid sequences.

19. The method of claim 14, wherein the VSV vector is oncolytic.

20. A composition comprising a recombinant vesicular stomatitis (VSV) vector expressing a tumor suppressor molecule.

21. The composition of claim 20, wherein the tumor suppressor molecule is p53, mutants, variants or complementary DNA (cDNA) sequences thereof.

22. The composition of claim 20, wherein the VSV vector is oncolytic.

23. The composition of claim 20, wherein the VSV vector is attenuated.

24. The composition of claim 20, wherein the VSV vector further comprises one or more foreign nucleic acid sequences or complementary DNA (cDNA) sequences thereof, for expressing one or more proteins or peptides.

25. The composition of claim 24, wherein the one or more foreign nucleic acid sequences or complementary DNA (cDNA) sequences thereof, comprise: interferons, ligands, immune related molecules, cytokines, chemokines, cellular products, cell surface expression or secretion products, cell signaling molecules, or combinations thereof.

26. The composition of claim 25, wherein immune related molecules comprise: T cell receptors or ligands thereof, co-stimulatory molecules or ligands thereof, immunoglobulins, cell surface expression markers or receptors thereof.

27. The composition of claim 26, wherein the interferons, cytokines, chemokines and cellular products comprise: interferon α-, β- and/or γ-, interleukins, growth factors, tumor necrosis factors, integrins, or combinations thereof.

28. The composition of claim 20, wherein the VSV further comprises one or more deletions or mutations in one or more VSV nucleic acid sequences.

29. A method of treating cancer comprising administering to a patient in need thereof, a therapeutically effective amount of a recombinant vesicular stomatitis (VSV) vector expressing a tumor suppressor molecule, wherein the tumor suppressor molecule is p53, mutants, variants or complementary DNA (cDNA) sequences thereof.

30. The method of claim 29, wherein the VSV vector is oncolytic.

31. The method of claim 29, wherein the VSV vector is attenuated.

32. The method of claim 29, wherein the VSV vector further comprises one or more foreign nucleic acid sequences or complementary DNA (cDNA) sequences thereof, for expressing one or more proteins or peptides.

33. The method of claim 32, wherein the one or more foreign nucleic acid sequences or complementary DNA (cDNA) sequences thereof, comprise: interferons, ligands, immune related molecules, cytokines, chemokines, cellular products, cell surface expression or secretion products, cell signaling molecules, or combinations thereof.

34. The method of claim 33, wherein immune related molecules comprise: T cell receptors or ligands thereof, co-stimulatory molecules or ligands thereof, immunoglobulins, cell surface expression markers or receptors thereof.

35. The method of claim 33, wherein the interferons, cytokines, chemokines and cellular products comprise: interferon α-, β- and/or γ-, interleukins, growth factors, tumor necrosis factors, integrins, or combinations thereof.

36. The method of claim 29, wherein the VSV further comprises one or more deletions or mutations in one or more VSV nucleic acid sequences.

37. A composition comprising an attenuated vesicular stomatitis (VSV) vector expressing a one or more oligonucleotides which modulate expression or function of target molecules.

38. The composition of claim 37, wherein the oligonucleotides comprises: dsRNA, siRNA, antisense RNA, RNA, enzymatic RNA or microRNA.

39. The composition of claim 37, wherein the VSV vector is oncolytic or kills abnormal cells.

40. The composition of claim 37, wherein the VSV vector is attenuated.

41. The composition of claim 37, wherein the VSV vector further comprises one or more foreign nucleic acid sequences or complementary DNA (cDNA) sequences thereof, for expressing one or more proteins or peptides, the one or more foreign nucleic acid sequences comprising: interferons, ligands, immune related molecules, cytokines, chemokines, cellular products, cell surface expression or secretion products, cell signaling molecules, or combinations thereof.

42. The composition of claim 41, wherein immune related molecules comprise: T cell receptors or ligands thereof, co-stimulatory molecules or ligands thereof, immunoglobulins, cell surface expression markers or receptors thereof.

43. The composition of claim 41, wherein the interferons, cytokines, chemokines and cellular products comprise: interferon α-, β- and/or γ-, interleukins, growth factors, tumor necrosis factors, integrins, or combinations thereof.

44. The composition of claim 37, wherein the VSV further comprises one or more deletions or mutations in one or more VSV nucleic acid sequences.

45. A recombinant vesicular stomatitis virus (VSV) vector comprising a nucleic acid encoding a cytokine, wherein said recombinant VSV vector exhibits greater oncolytic activity against a tumor cell than a wild-type VSV vector when contacted with the tumor cell.

46. The recombinant vesicular stomatitis virus (VSV) of claim 45, wherein the cytokine is an interferon beta nucleic acid sequence, and is inserted between the vesicular stomatitis virus vector genes, G and L and the VSV lacks G-protein function.

47. The recombinant vesicular stomatitis virus vector of claim 46, wherein the interferon beta nucleic acid sequence is complementary DNA (cDNA).

48. A recombinant vesicular stomatitis virus (VSV) vector consisting of
a) a nucleic acid comprising an interferon beta; and
b) a nucleic acid comprising an interferon gamma, IL-4, or IL-12.

49. The recombinant vesicular stomatitis virus vector of claim 48, wherein the interferon beta, interferon gamma, IL-4, or IL-12 nucleic acid sequences are complementary DNA (cDNA).


SEQUENCE LISTING (12 sequences)

SEQ ID NO: 1 - RNA, 11 nt, Vesicular stomatitis virus
  misc_RNA (1)...(11): Genomic RNA
uuuuuuucaua

SEQ ID NO: 2 - RNA, 10 nt, Vesicular stomatitis virus
  misc_RNA (1)...(10): Genomic RNA
gaunncuguu

SEQ ID NO: 3 - DNA, 35 bp, Artificial Sequence
  Synthetic construct
ggcactcgagatgggtctcaacccccagctagttg

SEQ ID NO: 4 - DNA, 36 bp, Artificial Sequence
  Synthetic construct
gccgtctagactacgagtaatccatttgcatgatgc

SEQ ID NO: 5 - DNA, 31 bp, Artificial Sequence
  Synthetic construct
ggcactcgagatggtgagcaagggcgaggag

SEQ ID NO: 6 - DNA, 36 bp, Artificial Sequence
  Synthetic construct
gcttgagctctagatctgagtccctacttgtacagc

SEQ ID NO: 7 - DNA, 39 bp, Artificial Sequence
  Synthetic construct
cttgtagactcgagtatggcttcgtaccccggccatcag

SEQ ID NO: 8 - DNA, 39 bp, Artificial Sequence
  Synthetic construct
gtattgtctgctagcgtgtttcagttagcctcccccatc

SEQ ID NO: 9 - DNA, 37 bp, Artificial Sequence
  Synthetic construct
tccatcctcgagcactatgaacaacaggtggatcctc

SEQ ID NO: 10 - DNA, 33 bp, Artificial Sequence
  Synthetic construct
aggtctgctagcctagttttggaagtttctggt

SEQ ID NO: 11 - DNA, 60 bp, Artificial Sequence
  Synthetic construct
gtatgaaaaaaactaacagatatcacgctcgagaattaattgctaggtatatgaaaaaaa

SEQ ID NO: 12 - DNA, 22 bp, Vesicular stomatitis virus
  misc_signal (2)...(12): Polyadenylation/transcription stop signal of the glycoprotein (G) gene
gtatgaaaaaaactaacagcaa